(19) United States
(12) Patent Application Publication
(10) Pub. No.: US 2007/0185080 A1
(43) Pub. Date: Aug. 9, 2007

(54) Pharmaceutical Compositions

(75) Inventors: Hans Eriksson, Sodertalje (SE); Martin Brecher, Wilmington, DE (US); Rohini Chitra, Wilmington, DE (US); Joan Shaw, Wilmington, DE (US); Marten Vagero, Sodertalje (SE); Ellis Wilson, Wilmington, DE (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, Sodertalje (SE)

(21) Appl. No.: 11/561,306

(22) Filed: Nov. 17, 2006

Related U.S. Application Data
(60) Provisional application No. 60/737,943, filed on Nov. 18, 2005.

(51) Int. Cl. A61K 31/554

(52) U.S. Cl. 514/211.13

(57) ABSTRACT

The present invention provides methods of treatment with a pharmaceutical composition, more particularly a sustained release pharmaceutical composition, comprising 11-[4-[2-(2-hydroxyethoxy)ethyl]-1-piperazinyl]dibenzo-[b,f][1,4]thiazepine or a pharmaceutically acceptable salt thereof, as well as new and improved methods for treating a variety of psychological disorders and conditions including, but not limited to, Mood Disorders and Anxiety Disorders and for treating one or more of the symptoms of these disorders.

### FIELD OF THE INVENTION

The present invention is directed, in part, to methods of treatment with a pharmaceutical composition, more particularly sustained release pharmaceutical compositions, comprising 11-[4-[2-(2-hydroxyethoxy)ethyl]-1-piperazinyl]dibenzo-[b,f][1,4]thiazepine or a pharmaceutically acceptable salt thereof, as well as new and improved methods for treating a variety of psychological disorders and conditions including, but not limited to, Mood Disorders and Anxiety Disorders and to treatment of symptoms of these disorders.

### BACKGROUND OF THE INVENTION

Quetiapine, the international nonproprietary name for 11-[4-[2-(2-hydroxyethoxy)ethyl]-1-piperazinyl]dibenzo-[b,f][1,4]thiazepine, is an atypical antipsychotic and is on the market as SEROQUEL® for the treatment of schizophrenia and the treatment of acute manic episodes associated with bipolar I disorder, as either monotherapy or adjunct therapy to lithium or divalproex.

A sustained release formulation of an active ingredient may be prepared using a gelling agent. While there are numerous sustained release formulations known in the art which utilize gelling agents, it has been found to be difficult to formulate sustained release formulations of soluble medicaments and gelling agents for several reasons. First, active ingredients which are soluble in water tend to generate a sustained release product which is susceptible to a phenomenon known as dose dumping. That is, release of the active ingredient is delayed for a time, but once release begins to occur the rate of release is very high. Moreover, fluctuations tend to occur in the plasma concentrations of the active ingredient which increases the likelihood of toxicity. Further, some degree of diurnal variation in plasma concentration of the active ingredient has also been observed. Finally, it has been found to be difficult to achieve the desired dissolution profiles or to control the rate of release of the soluble medicament. Sustained release formulations of quetiapine are disclosed in, for example, U.S. Pat. No. 5,948,437, the entire contents of which are incorporated herein by reference.

### SUMMARY OF THE INVENTION

The present invention provides methods of treating a patient suffering from or susceptible to a Mood Disorder or an Anxiety Disorder comprising administering a sustained release pharmaceutical composition comprising a pharmaceutically effective amount of 11-[4-[2-(2-hydroxyethoxy)ethyl]-1-piperazinyl]dibenzo-[b,f][1,4]thiazepine, or a pharmaceutically acceptable salt thereof, to the patient in need thereof.

The present invention also provides use of a sustained release composition comprising administering to a patient suffering from or susceptible to a Mood Disorder or an Anxiety Disorder a pharmaceutically effective amount of 11-[4-[2-(2-hydroxyethoxy)ethyl]-1-piperazinyl]dibenzo-[b,f][1,4]thiazepine, or a pharmaceutically acceptable salt thereof, to the patient in need thereof.

Also provided is use of a sustained release composition comprising a pharmaceutically effective amount of 11-[4-[2-(2-hydroxyethoxy)ethyl]-1-piperazinyl]dibenzo-[b,f][1,4]thiazepine, or a pharmaceutically acceptable salt thereof, for use in the manufacture of a medicament for use in the treatment of a patient suffering from or susceptible to a Mood Disorder or Anxiety Disorder.

### DETAILED DESCRIPTION OF THE INVENTION

As used herein, “Mood Disorder(s)” includes, but is not limited to, a) Depressive Disorders, including, but not limited to, Major Depressive Disorder (MDD) and Dysthymic Disorder; b) Bipolar Depression and/or Bipolar mania including, but not limited to, Bipolar I, including, but not limited to, those with manic, depressive or mixed episodes, and Bipolar II; c) Cyclothymic Disorder; and d) Mood Disorder Due to a General Medical Condition. In some embodiments, MDD may be present in elderly individuals having cerebrovascular damage.

As used herein, “Anxiety Disorder(s)” includes, but is not limited to, Panic Disorder without Agoraphobia, Panic Disorder with Agoraphobia, Agoraphobia without History of Panic Disorder, Specific Phobia, Social Phobia, Social Anxiety Disorder, Obsessive-Compulsive Disorder, Post-Traumatic Stress Disorder, Acute Stress Disorder, Generalized Anxiety Disorder (GAD) and GAD Due to a General Medical Condition.

Examples of definitions of the above-identified disorders can be found, for example, in the Diagnostic and Statistical Manual of Mental Disorders—Fourth Edition (DSM IV).

In some embodiments, the 11-[4-[2-(2-hydroxyethoxy)ethyl]-1-piperazinyl]dibenzo-[b,f][1,4]thiazepine, or a pharmaceutically acceptable salt thereof, is present in a sustained release form. The mean Cmax of any of the sustained release forms described herein is from about 10 ng/mL to about 700 ng/mL, or from about 50 ng/mL to about 500 ng/mL, or from about 100 ng/mL to about 250 ng/mL, and the area under the curve (AUC) is from about 100 ng*hr/mL to about 6000 ng*hr/mL, or from about 250 ng*hr/mL to about 5000 ng*hr/mL, or from about 500 ng*hr/mL to about 3000 ng*hr/mL, or from about 1000 ng*hr/mL to about 2000 ng*hr/mL. In some embodiments, the median Tmax of any of the sustained release forms described herein is from about 1.5 hours to about 7.5 hours, or from about 2.5 hours to about 5 hours, or from about 3 hours to about 4 hours. In some embodiments, the 11-[4-[2-(2-hydroxyethoxy)ethyl]-1-piperazinyl]dibenzo-[b,f][1,4]thiazepine, or a pharmaceutically acceptable salt thereof, is present in a sustained release form, which may comprise a polymer. In other embodiments, the 11-[4-[2-(2-hydroxyethoxy)ethyl]-1-piperazinyl]dibenzo-[b,f][1,4]thiazepine, or a pharmaceutically acceptable salt thereof, is not present within a sustained release form.

In some embodiments, the patient is in need of the treatment. In some embodiments, the patient will have been diagnosed as suffering from or susceptible to a Mood Disorder. In some embodiments, the patient does not suffer from schizophrenia.

In some embodiments, the patient will have been diagnosed as suffering from or susceptible to MDD. In some embodiments, the patient does not suffer from schizophrenia.

In still further embodiments, the patient will have been diagnosed or suffering from or susceptible to treatment-resistant MDD. In some embodiments, the patient does not suffer from schizophrenia.

In some embodiments, the treatment is monotherapy treatment with 11-[4-[2-(2-hydroxyethoxy)ethyl]-1-piperazinyl]dibenzo-[b,f][1,4]thiazepine.

In some embodiments, the treatment is maintenance treatment with 11-[4-[2-(2-hydroxyethoxy)ethyl]-1-piperazinyl]dibenzo-[b,f][1,4]thiazepine.

In some embodiments, the 11-[4-[2-(2-hydroxyethoxy)ethyl]-1-piperazinyl]dibenzo-[b,f][1,4]thiazepine is administered as part of adjunct therapy (in or not in the form of a sustained release form) with a selective serotonin reuptake inhibitor (SSRI) such as, for example, paroxetine, fluoxetine, sertaline, fluvoxamine, venlafaxine, nefazodone, and citalopram. In some embodiments, a therapeutically effective amount of the SSRI, or a pharmaceutically acceptable salt thereof, is administered with a pharmaceutical composition that comprises a sustained release form of 11-[4-[2-(2-hydroxyethoxy)ethyl]-1-piperazinyl]dibenzo-[b,f][1,4]thiazepine, or a pharmaceutically acceptable salt thereof.

In some embodiments, the 11-[4-[2-(2-hydroxyethoxy)ethyl]-1-piperazinyl]dibenzo-[b,f][1,4]thiazepine is administered as part of adjunct therapy (in or not in the form of a sustained release form) with a serotonin-norepinephrine reuptake inhibitor (SNRI) such as, for example, duloxetine.

In some embodiments, the adjunct therapy is for treatment of MDD or treatment-resistant MDD and comorbid anxiety.

In some embodiments, the adjunct therapy is for treatment of treatment-resistant MDD or MDD and comorbid anxiety.

The present invention also provides methods of relieving a symptom of depression in a patient suffering from or susceptible to a Mood Disorder comprising administering a therapeutically effective amount of 11-[4-[2-(2-hydroxyethoxy)ethyl]-1-piperazinyl]dibenzo-[b,f][1,4]thiazepine in a patient in need thereof, wherein 11-[4-[2-(2-hydroxyethoxy)ethyl]-1-piperazinyl]dibenzo-[b,f][1,4]thiazepine is administered to the patient once or twice daily. In some embodiments, the method excludes administering to the patient other compositions for relieving a symptom of depression in a patient suffering from or susceptible to a Mood Disorder.

The present invention also provides methods of relieving an anxiety symptom in a patient suffering from or susceptible to a Mood Disorder comprising administering a therapeutically effective amount of 11-[4-[2-(2-hydroxyethoxy)ethyl]-1-piperazinyl]dibenzo-[b,f][1,4]thiazepine in a patient in need thereof, wherein 11-[4-[2-(2-hydroxyethoxy)ethyl]-1-piperazinyl]dibenzo-[b,f][1,4]thiazepine is administered to the patient once or twice daily. In some embodiments, the method excludes administering to the patient other compositions for relieving an anxiety symptom in a patient suffering from or susceptible to a Mood Disorder.

The present invention also provides methods of relieving an anxiety symptom in a patient suffering from or susceptible to MDD comprising administering a therapeutically effective amount of 11-[4-[2-(2-hydroxyethoxy)ethyl]-1-piperazinyl]dibenzo-[b,f][1,4]thiazepine in a patient in need thereof, wherein 11-[4-[2-(2-hydroxyethoxy)ethyl]-1-piperazinyl]dibenzo-[b,f][1,4]thiazepine is administered to the patient once or twice daily. In some embodiments, the method excludes administering to the patient other compositions for relieving an anxiety symptom in a patient suffering from or susceptible to MDD.

The present invention also provides methods of improving the quality of life in a patient suffering from or susceptible to MDD comprising administering a therapeutically effective amount of 11-[4-[2-(2-hydroxyethoxy)ethyl]-1-piperazinyl]dibenzo-[b,f][1,4]thiazepine in a patient in need thereof, wherein 11-[4-[2-(2-hydroxyethoxy)ethyl]-1-piperazinyl]dibenzo-[b,f][1,4]thiazepine is administered to the patient once or twice daily.

The present invention also provides methods of treating a patient suffering from or susceptible to an Anxiety Disorder comprising administering to the patient a pharmaceutical composition comprising a pharmaceutically effective amount of 11-[4-[2-(2-hydroxyethoxy)ethyl]-1-piperazinyl]dibenzo-[b,f][1,4]thiazepine, or a pharmaceutically acceptable salt thereof. In some embodiments, the Anxiety Disorder is GAD.

In some embodiments, the patient is in need of the treatment. In some instances, the patient will have been diagnosed as suffering from or susceptible to an Anxiety Disorder. In still further embodiments, the patient will have been diagnosed as suffering from or susceptible to GAD. In some embodiments, the patient does not suffer from schizophrenia.

In some embodiments, the treatment is monotherapy treatment with 11-[4-[2-(2-hydroxyethoxy)ethyl]-1-piperazinyl]dibenzo-[b,f][1,4]thiazepine.

In some embodiments, the treatment is maintenance treatment with 11-[4-[2-(2-hydroxyethoxy)ethyl]-1-piperazinyl]dibenzo-[b,f][1,4]thiazepine.

In some embodiments, the 11-[4-[2-(2-hydroxyethoxy)ethyl]-1-piperazinyl]dibenzo-[b,f][1,4]thiazepine is administered as part of adjunct therapy (in or not in the form of a sustained release form) with a SSRI such as, for example, paroxetine, fluoxetine, sertaline, fluvoxamine, venlafaxine, nefazodone, and/or citalopram. In some embodiments, a therapeutically effective amount of the SSRI, or a pharmaceutically acceptable salt thereof, is administered in a pharmaceutical composition that comprises a sustained release form of 11-[4-[2-(2-hydroxyethoxy)ethyl]-1-piperazinyl]dibenzo-[b,f][1,4]thiazepine, or a pharmaceutically acceptable salt thereof.

In some embodiments, the 11-[4-[2-(2-hydroxyethoxy)ethyl]-1-piperazinyl]dibenzo-[b,f][1,4]thiazepine is administered as part of adjunct therapy (in or not in the form of a sustained release form) with a SNRI such as, for example, duloxetine.

The present invention also provides methods of relieving a symptom associated with GAD in a patient comprising administering a therapeutically effective amount of 11-[4-[2-(2-hydroxyethoxy)ethyl]-1-piperazinyl]dibenzo-[b,f][1,4]thiazepine in a patient in need thereof, wherein 11-[4-[2-(2-hydroxyethoxy)ethyl]-1-piperazinyl]dibenzo-[b,f][1,4]thiazepine is administered to the patient once or twice daily. In some embodiments, the method excludes administering to the patient other compositions for relieving a symptom associated with GAD. In some embodiments, the symptom treated is including, but not limited to, anxiety or relapse of an anxiety symptom with GAD.

The present invention also provides methods of ameliorating an undesirable psychological state in a mammal comprising administering to a patient in need thereof an effective, non-toxic dose of a sustained release form of 11-[4-[2-(2-hydroxyethoxy)ethyl]-1-piperazinyl]dibenzo-[b,f][1,4]thiazepine, or a pharmaceutically acceptable salt thereof, wherein the undesirable psychological state is Mood Disorder and/or Anxiety Disorder.

The present invention also provides methods of ameliorating an undesirable psychological state in a mammal comprising administering to a patient in need thereof an effective, non-toxic dose of a sustained release form of 11-[4-[2-(2-hydroxyethoxy)ethyl]-1-piperazinyl]dibenzo-[b,f][1,4]thiazepine, or a pharmaceutically acceptable salt thereof, wherein the undesirable psychological state is MDD and/or GAD.

In some embodiments, the 11-[4-[2-(2-hydroxyethoxy)ethyl]-1-piperazinyl]dibenzo-[b,f][1,4]thiazepine, or a pharmaceutically acceptable salt thereof, or any formulation thereof, can be used in combination with one or more SSRIs such as, for example, sertraline to treat Post-Traumatic Stress Disorder.

The compound, 11-[4-[2-(2-hydroxyethoxy)ethyl]-1-piperazinyl]-dibenzo[b,f][1,4]thiazepine (also known as quetiapine; see Formula I below), and its pharmaceutically acceptable salts exhibit useful antidopaminergic activity. It is a compound of particular interest because it can be used as an antipsychotic agent with a substantial reduction in the potential to cause side effects such as acute dystonia, acute dyskinesia, pseudo-Parkinsonism and tardive dyskinesia, which side-effects can result from the use of other antipsychotics or neuroleptics.

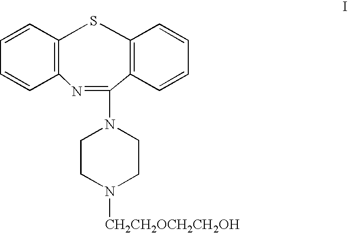

Sustained release formulations of quetiapine are disclosed in U.S. Pat. No. 5,948,437, the entire contents of which are incorporated herein by reference. Further, a sustained release formulation may be used in treating GAD, MDD and associated symptoms.

Another embodiment of the invention provides quetiapine as an effective treatment of MDD or GAD while exhibiting fewer or less severe adverse effects. Another embodiment of the invention is a once or twice daily dosing regimen of quetiapine for the treatment of conditions and symptoms disclosed herein. Yet another embodiment of the invention is a method of treating GAD or MDD with quetiapine as a monotherapy. Another embodiment of the invention is a method of treating GAD or MDD with quetiapine as part of combination therapy. Another embodiment of the invention is a method of treating GAD or MDD with quetiapine as adjunctive therapy. Another embodiment of the invention is the use of quetiapine to treat GAD or MDD which results in an improvement in the quality of life of the patient suffering from GAD or MDD or associated symptoms.

Symptoms of GAD include, but are not limited to, excessive worry and anxiety, difficulty controlling worry, restlessness or feeling keyed up or on edge, being easily fatigued, difficulty concentrating or mind going blank, irritability, muscle tension, or sleep disturbance.

Pharmaceutical compositions of the invention are useful, for example, in maintaining improvement of depressive symptoms in patients with MDD, in the treatment of anxiety symptoms in patients with MDD, in demonstrating improvements in the quality of sleep in patients with MDD, in reducing suicidal ideation in patients with MDD, and generally improving the quality of life in patients with MDD.

The present invention also provides a sustained release formulation of quetiapine wherein once daily administration of the formulation is at least as effective as a SNRI in the treatment of Mood Disorders including but not limited to Depressive Disorders such as MDD.

The present invention also provides once daily administration of a formulation described herein wherein the formulation is at least as effective as a SNRI in: 1) reducing anxiety symptoms; 2) improving sleep onset in patients; 3) improving sleep maintenance; 4) reducing suicide ideation; 5) improving somatic symptoms including, but not limited to, back pain, headache, muscle pain, unspecified pain, abdominal pain, and chest pain in patients with depression in need of such treatment; 6) improving quality of life; and 7) improving patient satisfaction in patients with depression. Fasting glucose and lipids may not be significantly elevated and serious discontinuation symptoms may not be present.

The invention also provides maintenance treatment of MDD in patients who have responded to acute treatment. Clinical studies may indicate efficacy of a sustained release formulation of quetiapine compared to placebo in increasing time from randomization to relapse of a depressed event in patients with MDD. Quetiapine once daily may maintain improvement of depressive symptoms in patients with MDD during long-term treatment. Quetiapine once daily may treat anxiety symptoms, reduce suicidal ideation and improve quality of life in patients with MDD during long-term treatment. Further, a sustained release formulation of quetiapine may be safe and well tolerated in long-term treatment of patients with MDD.

The compositions and dosage forms of the invention are designed to deliver an effective amount of quetiapine or a pharmaceutically acceptable salt thereof to a mammal, preferably a human. The clinical dosage range for alleviation of Mood Disorders and Anxiety Disorders may be provided in an amount up to about 800 mg per day. Since the dosage should be tailored to the individual patient, a single daily dosage may be applicable, but division of the daily dose into 2 or 3 portions may also possible.

Another embodiment of the invention is the treatment of GAD with quetiapine. Pharmaceutical compositions are provided herein to reduce the risk of relapse of anxiety symptoms in patients with GAD. Pharmaceutical compositions of the invention are provided to improvement the quality of sleep in patients with GAD. Another embodiment of the invention is to provide a formulation of quetiapine for once daily administration to maintain efficacy in patients in need thereof. Another embodiment of the invention is to provide a once-daily administered formulation of quetiapine that is well-tolerated long term in patients with GAD.

Quetiapine may be efficacious and safe in the acute term treatment of patients with GAD. Sustained release formulations of the invention may demonstrate an anxiety effect in patients within the first two weeks. Further, formulations of the invention may not demonstrate serious discontinuation symptoms. Quetiapine may be effective in GAD across anxiety symptoms. Compositions of the invention may be associated with lower levels of sexual dysfunction compared SSRIs including, but not limited to, escitalopram and paroxetine, may be associated with lower levels of nausea and vomiting compared to SSRIs including, but not limited to, escitalopram or paroxetine. When dosed once daily, compositions of the invention may improve sleep onset and sleep maintenance in depression, reduce suicide ideation in patients with depression and improving somatic symptoms such as back pain, headache, muscle pain, unspecified pain, abdominal pain, chest pain, in patients with depression. Sustained release formulations of the invention may also improve the quality of life of patients with depression and have a response rate in depression. Compositions of the invention when dosed once daily may also achieve remission and reduce anxiety symptoms in patients with depression.

The present invention may also contain or be administered with a therapeutically effective amount of a SSRI or SNRI in addition to a therapeutically effective amount of quetiapine or a pharmaceutically acceptable salt thereof, and a pharmaceutically acceptable carrier for oral administration. SSRIs include, but are not limited to, paroxetine (PAXIL®), fluoxetine (PROZAC®), sertaline (ZOLOFT®), fluvoxamine (LUVOX®), venlafaxine (EFFFEXOR®), escitalopram oxalate (LEXAPRO®), and nefazodone (SERZONE®), as well as any optically pure isomers or metabolites of any of these compounds. SNRIs include, but are not limited to, duloxetine.

The present invention may also be efficacious in combination with an anti-depressant in the treatment of MDD in patients who have inadequate response to an anti-depressant. Sustained release formulations of the present invention when administered once daily in combination with an anti-depressant may be more effective than an anti-depressant alone in reducing anxiety symptoms, improving sleep onset, reducing suicide ideation and improving somatic symptoms in depression. Somatic symptoms include, but are not limited to, back pain, headache, muscle pain, unspecified pain, abdominal pain and chest pain in patients with depression. Sustained release formulations of the invention administered once daily in combination with an anti-depressant may be more effective than an anti-depressant alone in improving the quality of life of patients with depression. Further compositions of the invention in combination with an anti-depressant may not have serious discontinuation symptoms. Sustained release formulations of the invention administered once daily in combination with an anti-depressant up to 300 mg/day may be well tolerated in patients with depression.

Another aspect of the invention provides quetiapine or a pharmaceutically acceptable salt thereof as a potentiation of SSRI and SNRI treatment in MDD with comorbid anxiety. SSRIs of this aspect of the invention include but are not limited to citalopram, paroxetine, venlafaxine, fluoxetine, sertraline. The preparation, physical properties and beneficial pharmacological properties of 11-[4-[2-(2-hydroxyethoxy)ethyl]-1-piperazinyl]dibenzo[b,f][1,4]-thiazepine, and its pharmaceutically acceptable salts are described in published European Patents EP 240,228 and 282,236 as well as in U.S. Pat. No. 4,879,288, the entire contents of which are incorporated herein by reference.

In some embodiments of the invention, the sustained release formulation comprises a gelling agent such as, for example, hydroxypropyl methylcellulose, and 11-[4-[2-(2-hydroxyethoxy)ethyl]-1-piperazinyl]dibenzo-[b,f][1,4]thiazepine, or pharmaceutically acceptable salt thereof, together with one or more pharmaceutically acceptable excipients. In some embodiments, the sustained release formulation comprises a hydrophilic matrix comprising a gelling agent, such as hydroxypropyl methylcellulose, and 11-[4-[2-(2-hydroxyethoxy)ethyl]-1-piperazinyl]dibenzo-[b,f][1,4]thiazepine, or a pharmaceutically acceptable salt thereof, together with one or more pharmaceutically acceptable excipients.

As used herein, “gelling agent” means any substance, particularly a hydrophilic substance, which forms a gel when in contact with water and, thus, includes substances such as, for example, hydroxypropyl methylcellulose, hydroxypropylcellulose, hydroxymethylcellulose, hydroxyethylcellulose, hydroxypropyl ethylcellulose, methylcellulose, ethylcellulose, carboxyethylcellulose, carboxymethyl hydroxyethylcellulose, carbomer, sodium carboxymethylcellulose, polyvinylpyrrolidone, and the like, or any subgroup thereof, or any mixture thereof. In some embodiments, the gelling agent is hydroxypropyl methylcellulose.

The amount of gelling agent, such as hydroxypropyl methylcellulose, is selected such that the active ingredient is released from the formulation, in a controlled fashion, over a period of about 4 hours or longer,or over a period of about 8 hours or longer, or over a period of between about 8 and about 24 hours, so that at least about 60% of the active ingredient has been released at the end of this period.

The gelling agent, such as hydroxypropyl methylcellulose, is conveniently present in about 5 to about 50% (by weight), or about 5 to about 40%, or about 8 to about 35%, or about 10 to about 35%. It is generally suitable that the gelling agent, such as hydroxypropyl methylcellulose, is present in about 10 to about 30%, or about 15 to about 30%.

The hydroxypropyl methylcellulose may contain more than one grade of polymer and is commercially available under several trademarks, e.g. METHOCEL® E, F, J and K from the Dow Chemical Company, U.S.A. and METALOSE® SH from Shin-Etsu, Ltd., Japan. The various grades available under a particular trademark represent differences in methoxy and hydroxypropoxy content as well as in viscosity. The methoxy content ranges from about 16.5% to about 30% by weight, the hydroxypropoxy content ranges from about 4% to about 32% by weight and the viscosities of a 2% aqueous solution at 20° C. range from about 3 cps to about 100,000 cps. For example, the hydroxypropyl methylcellulose may compris: a) a polymer with a viscosity of about 40 to about 60 cps (in particular, about 50 cps), a methoxy content of about 28% to about 30% by weight, and a hydroxypropoxy content of from about 7% to less than about 9% by weight; b) a polymer with a viscosity of about 3,500 to about 5,600 cps (in particular, about 4,000 cps), a methoxy content of about 28% to about 30% by weight, and a hydroxypropoxy content of about 7% to about 12% by weight; c) a polymer with a viscosity of about 80 to about 120 cps (in particular, about 100 cps), a methoxy content of about 19% to about 24% by weight, and a hydroxypropoxy content of from about 7% to less than about 9% by weight; or d) a polymer with a viscosity of about 3500 to about 5600 cps (in particular, about 4,000 cps), a methoxy content of about 19% to about 24% by weight and a hydroxypropoxy content of about 7% to about 12% by weight, or any mixture thereof. In some embodiments, the hydroxypropyl methylcellulose is selected from the group consisting of a)-d) or any mixture thereof as described above with the proviso that if the formulation contains a hydroxypropyl methylcellulose described under d) above, the total amount of hydroxypropyl methylcellulose present in the formulation must be greater than about 25.8% by weight.

In one embodiment the hydroxypropyl methylcellulose comprises about 8% to about 12% of a polymer having a viscosity of about 4,000 cps, and preferably about 5% to about 10%. In a further embodiment hydroxypropyl methylcellulose comprises about 10% to about 35% of a polymer having a viscosity of about 50 cps, and preferably about 10% to about 15%.

In another embodiment, the hydroxypropyl methylcellulose comprises about 15% of a polymer having a viscosity of about 50 cps, and optionally about 5% of a hydroxypropyl methylcellulose polymer having a viscosity of about 4,000 cps.

In particular the 11-[4-[2-(2-hydroxyethoxy)-ethyl]-1-piperazinyl]-dibenzo[b,f][1,4]thiazepine, or pharmaceutically acceptable salt thereof (such as the hemifumarate salt), is present in about 10% to about 90% by weight, or about 20% to about 80% by weight, or about 35% to about 65% by weight, or about 40% to about 60% by weight, or about 43.2% to about 57.6% by weight.

The formulation will, in general, contain one or more excipients. Such excipients include, but are not limited to: 1) diluents such as lactose, microcrystalline cellulose, dextrose, mannitol, sucrose, sorbitol, gelatin, acacia, dicalcium phosphate, tricalcium phosphate, monocalcium phosphate, sodium phosphate, sodium carbonate and the like, preferably lactose and microcrystalline cellulose; 2) lubricants such as stearic acid, zinc, calcium or magnesium stearate and the like; 3) binders such as sucrose, polyethylene glycol, povidone (polyvinylpyrrolidone), corn or maize starch, pregelatinized starch and the like; 4) colorants such as ferric oxides, FD&C dyes, lakes and the like; 5) flavoring agents; and 6) pH modifiers which include suitable organic acids or alkali metal (e.g. lithium, sodium or potassium) salts thereof, such as benzoic acid, citric acid, tartaric acid, succinic acid, adipic acid and the like or the corresponding alkali metal salts thereof, preferably the alkali metal salts of such acids and in particular the sodium salt of citric acid (i.e., sodium citrate). The excipient(s) will, in general, be present in about 10% to about 90% by weight, or about 20% to about 80% by weight, or about 20% to about 45% by weight, or about 20% to about 40% by weight, or about 22.4% to about 36.8% by weight. The formulation may contain one or more pharmaceutically acceptable excipients selected from the group consisting of microcrystalline cellulose, lactose, magnesium stearate, sodium citrate and povidone. In particular, the formulation may contain one or more of a) microcrystalline cellulose, such as in the amount of about 4% to about 20% by weight; b) lactose, such as in the amount of about 5% to about 20% by weight; c) magnesium stearate, such as in the amount of about 1% to about 3% by weight; d) about 10% to about 30% by weight, or about 12.5% to about 25%, or about 12.5% by weight of sodium citrate; and e) about 1% to about 15% by weight, or about 4% to about 6% by weight, or about 5% by weight of povidone (polyvinylpyrrolidone).

According to the present invention there is also provided a sustained release formulation comprising a gelling agent, such as hydroxypropyl methylcellulose, and 11-[-4-[2-(2-hydroxyethoxy)ethyl]-1-piperazinyl]dibenzo-[b,f][1,4]thiazepine, or a pharmaceutically acceptable salt thereof, together with one or more pharmaceutically acceptable excipients wherein one of the excipients is a pH modifier.

According to the present invention there is also provided a sustained release formulation comprising 11-[4-[2-(2-hydroxyethoxy)ethyl]-1-piperazinyl]-dibenzo[b,f][1,4]thiazepine, or a pharmaceutically acceptable salt thereof, as active ingredient and about 5% to about 40% of hydroxypropyl methylcellulose, together with one or more pharmaceutically acceptable excipients.

According to the present invention there is also provided a sustained release formulation comprising about 35% to about 65% of 11-[4-[2-(2-hydroxyethoxy)-ethyl]-1-piperazinyl]dibenzo[b,f][1,4]thiazepine, or a pharmaceutically acceptable salt thereof, as active ingredient and about 5% to about 40% by weight of hydroxypropyl methylcellulose, together with one or more pharmaceutically acceptable excipients.

According to the present invention there is also provided a sustained release formulation comprising about 35% to about 65% of 11-[4-[2-(2-hydroxyethoxy)-ethyl]-1-piperazinyl]dibenzo[b,f][1,4]thiazepine, or a pharmaceutically acceptable salt thereof, as active ingredient and about 15% to about 30% of hydroxypropyl methylcellulose, together with about 20% to about 45% of one or more pharmaceutically acceptable excipients.

According to the present invention there is also provided a sustained release formulation comprising about 35% to about 65% of 11-[4-[2-(2-hydroxyethoxy)-ethyl]-1-piperazinyl]-dibenzo[b,f][1,4]thiazepine as active ingredient, or a pharmaceutically acceptable salt thereof, about 5% to about 40% by weight of hydroxypropyl methylcellulose, about 4% to about 12% microcrystalline cellulose, about 8% to about 20% lactose, and the remainder being one or more further pharmaceutically acceptable excipients. Such further excipients may include components which act as a lubricant (for example, magnesium stearate) during the manufacture of the formulation or dosage form.

According to the present invention there is also provided a sustained release formulation comprising about 5% to about 40% by weight of a hydroxypropyl methylcellulose selected from the group consisting of: a) a hydroxypropyl methylcellulose having a viscosity of about 40 to 60 cps, a methoxy content of about 28% to about 30% by weight, and a hydroxypropoxy content of from about 7% to less than about 9% by weight; b) a hydroxypropyl methylcellulose having a viscosity of about 3,500 to about 5,600 cps, a methoxy content of about 28% to about 30% by weight, and a hydroxypropoxy content of about 7% to about 12% by weight; c) a hydroxypropyl methylcellulose having a viscosity of about 80 to about 120 cps, a methoxy content of about 19% to about 24% by weight, and a hydroxypropoxy content of from about 7% to less than about 9% by weight; or d) a hydroxypropyl methylcellulose having a viscosity of about 3,500 to about 5,600 cps, a methoxy content of about 19% to about 24% by weight, and a hydroxypropoxy content of about 7% to about 12% by weight, or any mixture thereof, about 35% to about 65% by weight of 11-[4-[2-(2-hydroxyethoxy)ethyl]-1-piperazinyl]-dibenzo[b,f][1,4]thiazepine or a pharmaceutically acceptable salt thereof, and about 20% to about 45% by weight of one or more pharmaceutically acceptable excipients; with the proviso that if the formulation contains a hydroxypropyl methylcellulose described under d) above the total amount of hydroxypropyl methylcellulose present in the formulation must be greater than about 25.8% by weight.

Other formulations within the ambit of this latter group are those comprising about 8% to about 35% by weight of a hydroxypropyl methylcellulose selected from the group consisting of: a) a hydroxypropyl methylcellulose having a viscosity of about 40 to about 60 cps, a methoxy content of about 28% to about 30% by weight, and a hydroxypropoxy content of about 7% to less than about 9% by weight; b) a hydroxypropyl methylcellulose having a viscosity of about 3,500 to about 5,600 cps, a methoxy content of about 28% to about 30% by weight, and a hydroxypropoxy content of about 7% to about 12% by weight; c) a hydroxypropyl methylcellulose having a viscosity of about 80 to about 120 cps, a methoxy content of about 19% to about 24% by weight, and a hydroxypropoxy content of about 7% to less than about 9% by weight; or d) a hydroxypropyl methylcellulose having a viscosity of about 3,500 to about 5,600 cps, a methoxy content of about 19% to about 24% by weight, and a hydroxypropoxy content of about 7% to about 12% by weight, or any mixture thereof, about 35% to about 65% by weight of 11-[4-[2-(2-hydroxyethoxy)ethyl]-1-piperazinyl]dibenzo[b,f][1,4]thiazepine or a pharmaceutically acceptable salt thereof, and about 20% to about 45% by weight of one or more pharmaceutically acceptable excipients.

Still other formulations within the ambit of this latter group are those comprising about 10% to about 30% by weight of a hydroxypropyl methylcellulose selected from the groups a)-d) or any mixture thereof as described above; about 40% to about 60% by weight of 11-[4-[2-(2-hydroxyethoxy)ethyl]-1-piperazinyl]dibenzo[b,f][1,4]-thiazepine or a pharmaceutically acceptable salt thereof, and about 20% to about 40% by weight of one or more pharmaceutically acceptable excipients.

Suitable formulations within this latter group are those comprising about 15% to about 30% by weight of a hydroxypropyl methylcellulose selected from the groups a)-d) or any mixture thereof as described above; about 43.2% to about 57.6% by weight of 11-[4-[2-(2-hydroxyethoxy)ethyl]-1-piperazinyl]dibenzo[b,f]-[1,4]thiazepine or a pharmaceutically acceptable salt thereof, and about 22.4% to about 36.8% by weight of one or more pharmaceutically acceptable excipients.

Particularly suitable formulations within this latter group are those comprising about 15% to about 30% by weight of a hydroxypropyl methylcellulose selected from the groups a)-d) or any mixture thereof as described above; about 43.2% to about 57.6% by weight of 11-[4-[2-(2-hydroxyethoxy)ethyl]-1-piperazinyl]dibenzo[b,f]-[1,4]thiazepine or a pharmaceutically acceptable salt thereof, and about 22.4% to about 36.8% by weight of one or more pharmaceutically acceptable excipients selected from the group consisting of i) about 4% to about 12% by weight of microcrystalline cellulose; ii) about 5% to about 20% by weight of lactose; iii) about 1% to about 3% by weight of magnesium stearate; iv) about 10% to about 30% by weight of sodium citrate; and v) about 1% to about 15% by weight of povidone (polyvinylpyrrolidone).

In the above-described formulations, the 11-[4-[2-(2-hydroxy-ethoxy)ethyl]-1-piperazinyl]dibenzo[b,f][1,4]-thiazepine may be in the form of a hemifumarate salt which form has an equilibrium solubility in water at 20° C. of 3.29 mg/mL.

Formulations defined in the accompanying Examples are provided as a further feature of the present invention. However, it should be understood that the examples are for illustrative purposes only and are not intended to limit the invention to the examples expressly disclosed.

The formulations of the present invention may be prepared by conventional technology well known to those skilled in the art such as wet granulation, direct compression, dry compaction (slugging) and the like. Thus, for example, the active ingredient 11-[4-[2-(2-hydroxyethoxy)ethyl]-1-piperazinyl]-dibenzo[b,f]-[1,4]thiazepine, or a pharmaceutically acceptable salt thereof, a gelling agent such as hydroxypropyl methylcellulose, and other excipients are mixed together to form the sustained release formulations of the present invention. The active ingredient 11-[4-[2-(2-hydroxyethoxy)ethyl]-1-piperazinyl]-dibenzo[b,f][1,4]thiazepine, or a pharmaceutically acceptable salt thereof, a gelling agent such as hydroxypropyl methylcellulose, and other excipients can be mixed together to form a mixture suitable for compressing into tablets, which mixture is then compressed to form tablets or is filled into capsules.

The mixing process is can be carried out by mixing the components, wet granulating the mixed components, drying the mixture, milling the dried mixture, blending the mixture with a lubricant such as magnesium stearate and compressing the blended mixture to form tablets or filling the blended mixture into capsules.

A suitable process for preparing the formulations of the invention comprises the following steps:

a) mixing 11-[4-[2-(2-hydroxyethoxy)ethyl]-1-piperazinyl]dibenzo[b,f][1,4]-thiazepine, or a pharmaceutically acceptable salt thereof, a gelling agent such as hydroxypropyl methylcellulose, and other excipients;

b) wet granulating the mixed components;

c) drying the mixture;

d) milling the dried mixture;

e) blending the mixture with a lubricant such as magnesium stearate; and

f) compressing the blended mixture to form tablets.

The dosage forms may be coated with one or more coatings as is well known in the art such as, for example, shellac, zein, hydroxypropyl cellulose, hydroxypropyl methylcellulose, ethyl cellulose, polymethacrylates, polyvinyl acetate phthalate, cellulose acetate phthalate, triacetin, dibutyl sebacate, a mixture of polyethylene glycol, titanium dioxide and hydroxypropyl methylcellulose, and the like, or any subgroup thereof.

The sustained release properties of the formulation of the present invention may be demonstrated by monitoring the dissolution of the active ingredient. The dissolution of the active ingredient may be monitored using standard procedures well known to those skilled in the art (e.g., the dissolution test procedures, such as the Rotating Basket Method (Apparatus I) or Paddle Method (Apparatus II), disclosed in the U.S. Pharmacopeia (USP)). Such procedures include those in which the formulation is immersed in an aqueous medium such as water or hydrochloric acid and aliquots of the medium are withdrawn at various time points over a period of 24 hours. The aliquots are analyzed using high pressure liquid chromatography (HPLC) with UV detection to determine the concentration of dissolved active ingredient using standard methodology. In a particular example, a tablet is immersed in about 900 mL of water and the dissolution profile determined. In another particular example, the dissolution profile is determined by the Rotating Basket method by immersing a tablet in 750 mL of 0.1N HCl for 2 hours at a speed of 100 rpm and then adding 250 mL of 0.2 M phosphate buffer to the dissolution media to afford a pH of 6.2.

The formulation may release the active ingredient in a controlled manner over a period of up to about 8 hours or longer. For example, the formulation described in Example 2 below released about 90% of the active ingredient over about 16 hours, and the formulation described in Example 1 released about 90% of the active ingredient over a period of about 8 hours.

The plasma concentration versus time profiles of the active ingredient can be obtained utilizing the following procedure. Thirty-two patients are assigned to either Group A or Group B with 16 patients in each group. After a 2-day drug-free period (days 1 and 2), all patients are given oral doses of the immediate release formulation of example 12 twice daily for a 9-day period (days 3 through 11) with fixed step-wise increases in dose from 25 to 200 mg. Starting on day 12, patients can begin a randomized treatment sequence within their respective groups (Group A or B). Group A patients can follow a treatment sequence that includes one of each of the following formulations of the active ingredient administered according to the sequence randomized: two 100 mg tablets of the immediate release formulation of example 12 while fasting administered every 12 hours (Treatment 1), one 400 mg tablet of the formulation of example 2 while fasting (Treatment 2) and one 400 mg tablet of the formulation of example 2 with a meal (Treatment 3). Group B patients are randomized to a treatment sequence that includes one of each of the following formulations of the active ingredient administered according to the sequence randomized: two 100 mg tablets of the immediate release formulation of example 12 while fasting administered every 12 hours (Treatment 1), one 400 mg tablet of the formulation of example 1 while fasting (Treatment 4) and one 400 mg tablet of the formulation of example 1 with a meal (Treatment 5). On days 12, 16 and 20 patients can receive trial treatment according to their assigned treatment sequences. On the evenings of days 13 and 17, patients receive 200 mg doses of the immediate release formulation of example 12 and on days 14, 15, 18 and 19 the patients receive 200 mg dose of the immediate release formulation of example 12 twice daily. Blood samples are taken from each subject on days 3, 10, 11, 14, 15, 18 and 19 before the morning dose. On days, 12, 16 and 20 blood samples are taken from each subject immediately before dose administration and at specified time intervals from immediately after dose administration to 36 hours after dose administration. The concentration of the active ingredient in the blood samples is quantified using liquid-liquid extraction and high performance liquid chromatography with ultraviolet absorbance detection.

| | |
|---|---|
| Group A | Group B |

| Example No. | AUC₀₋₂₄ | Cₘₐₓ | AUC₀₋₂₄ | Cₘₐₓ |
|---|---|---|---|---|
| 1 | — | — | 4886 | 565 |
| 2 | 5609 | 433 | — | — |
| 12 | 5347 | 703 | 4818 | 563 |

The dose of the compound of the present invention which is administered will necessarily be varied according to principles well known in the art taking account of the route of administration, the duration of treatment, the severity of the psychotic condition, the size and age of the patient, the potency of the active component and the patient's response thereto. An effective dosage amount of the active component can thus readily be determined by the clinician after a consideration of all criteria and using his best judgment on the patient's behalf. In general, the compound will be administered to a warm blooded animal (such as man) so that an effective dose is received, generally a daily dose in the range of about 0.0 1 to about 40 mg/kg body weight. For example, when administered orally, it is generally administered in the range of about 0.1 to about 40 mg/kg body weight. The compound of the present invention can be administered in about a 25, 50, 200, 300 or 400 mg strength.

The formulation of the present invention will, in general, be in the form of a unit dosage form, and, in particular, the formulation will be in the form of a tablet.

Each of the sustained release pharmaceutical compositions described herein can be used in the manufacture of a medicament for use in the treatment of a patient suffering from or susceptible to a Mood Disorder or an Anxiety Disorder.

It will be apparent to those skilled in the art that the formulation can be co-administered with other therapeutic or prophylactic agents and/or medicaments that are not medically incompatible therewith. The formulation of the present invention does not, in general, show any indication of overt toxicity in laboratory test animals at several multiples of the minimum effective dose of the active ingredient.

The invention is further illustrated by the following non-limiting Examples in which temperatures are expressed in degrees Celsius. The compound 11-[4-[2-(2-hydroxyethoxy)ethyl]-1-piperazinyl]dibenzo[b,f]]1,4]-thiazepine, and its pharmaceutically acceptable salts, may be prepared as described in published European Patents EP 240,228 or 282,236 as well as in U.S. Pat. No. 4,879,288, the entire contents of which are herein incorporated by reference.

In order that the invention disclosed herein may be more efficiently understood, examples are provided below. It should be understood that these examples are for illustrative purposes only and are not intended to limit the invention to the examples expressly disclosed.

### EXAMPLES

### Example 1

### Preparation of Tablets

The following process is used to prepare tablets. 11-[4-[2-(2-hydroxyethoxy)ethyl]-1-piperazinyl]-dibenzo-[b,f]]1,4]thiazepine hemifumarate (3453.8 g), lactose (1144.7 g), microcrystalline cellulose (381.5 g) and METHOCEL® E50LV (900 g) are blended in a planetary mixer for approximately 3 minutes.

The mixture is wet granulated in a planetary mixer using purified water. The wet mass is dried in a fluidized bed drier at about 65° C. until the loss on drying is less than about 3% as measured by a moisture balance. The dried granulation is milled using a hammer type or similar mill operating at fast speed, knives forward with suitable screen (e.g. 20 to 40 mesh). Magnesium stearate is passed through an appropriate screen (e.g. 20 to 40 mesh). The dry granulated material is blended for approximately 3 minutes in a conventional blender (for example, Patterson-Kelley Twin Shell) with the screened magnesium stearate. The blended mixture is compressed into tablets using a conventional rotary tablet press (for example, Kilian LX-21).

TABLE 1

| | mg/Tablet | % of Tablet |
|---|---|---|
| Active ingredient (a) | 460.51 | 57.6 |
| Lactose NF | 152.62 | 19.1 |
| Microcrystalline Cellulose NF | 50.87 | 6.3 |
| METHOCEL ® E50LV Premium (b) | 120.00 | 15.0 |
| Purified water (c) | q.s | — |
| Magnesium stearate NF | 16.00 | 2.0 |
| (a) The active ingredient is 11-[4-[2-(2-hydroxyethoxy)ethyl]-1-piperazinyl]-dibenzo[b,f]][1,4]thiazepine hemifumarate | | |
| (b) METHOCEL ® E50LV Premium is hydroxypropyl methylcellulose with a viscosity of 40-60 cps, a methoxy content of 28% to 30% by weight and a hydroxypropoxy content of 7% to 12% by weight which may be obtained from The Dow Chemical Company, Michigan, USA. This product meets the specifications for HPMC 2910 USP. Note that the particular METHOCEL ® E50LV Premium in this example has a viscosity of 48 cps, a methoxy content of 28.9% by weight and a | | |
| # hydroxypropoxy content of less than 9.0% by weight (i.e. 8.0%). | | |

(c) Added but not retained.

### Example 2

### Preparation of Tablets

The procedure described in Example 1 is repeated using METHOCEL® E50LV and METHOCEL® E4M in place of METHOCEL® E50LV to afford tablets of the following composition.

TABLE 2

| | mg\Tablet | % of Tablet |
|---|---|---|
| Active ingredient (a) | 460.51 | 57.6 |
| Lactose NF | 81.74 | 10.2 |
| Microcrystalline Cellulose NF | 81.75 | 10.2 |
| METHOCEL E50LV Premium (b) | 120.00 | 15.0 |
| METHOCEL E4M Premium CR (d) | 40.00 | 5.0 |
| Purified water (c) | q.s | — |
| Magnesium stearate NF | 16.00 | 2.0 |
| (a) The active ingredient is 11-[4-[2-(2-hydroxyethoxy)ethyl]-1-piperazinyl]-dibenzo[b,f][1,4]thiazepine hemifumarate | | |
| (b) METHOCEL ® E50LV Premium is hydroxypropyl methylcellulose with a viscosity of 40-60 cps, a methoxy content of 28% to 30% by weight and a hydroxypropoxy content of 7% to 12% by weight which may be obtained from The Dow Chemical Company, Michigan, USA. This product meets the specifications for HPMC 2910 USP. Note that the particular METHOCEL ® E50LV Premium in this example has a viscosity of 48 cps, a methoxy content of 28.9% by | | |
| # weight and a hydroxypropoxy content of less than 9.0% by weight (i.e. 8.0%). | | |
| (c) Added but not retained. | | |
| (d) METHOCEL ® E4M Premium CR is hydroxypropyl methylcellulose with a viscosity of 3,500 to 5,600 cps, a methoxy content of 28% to 30% by weight and a hydroxypropoxy content of 7% to 12% by weight which may be obtained from The Dow Chemical Company, Michigan, USA. This product meets the specifications for HPMC 2910 USP. Note that the particular METHOCEL ® E4M Premium CR in this example has a viscosity of 4364 cps, a methoxy | | |
| # content of 28.5% by weight and a hydroxypropoxy content of 7.8% by weight. | | |

### Example 3

### Preparation of Composition

Following a procedure similar to that described in Example 1, tablets of the following composition can be prepared.

TABLE 3

| | mg\Tablet | % of Tablet |
|---|---|---|
| Active ingredient (a) | 345.38 | 43.2 |
| Lactose NF | 49.31 | 6.2 |
| Microcrystalline Cellulose NF | 49.31 | 6.2 |
| Sodium citrate | 100.00 | 12.5 |
| METHOCEL ® K100LV Premium CR (b) | 200.00 | 25.0 |
| METHOCEL ® K4M Premium CR (c) | 40.00 | 5.0 |
| Purified water (d) | q.s | — |
| Magnesium stearate NF | 16.00 | 2.0 |

(a) The active ingredient is 11-[4-[2-(2-hydroxyethoxy)ethyl]-1-piperazinyl] dibenzo[b,f][1,4]thiazepine hemifumarate
(b) METHOCEL ® K100LV Premium CR is hydroxypropyl methylcellulose with a viscosity of 80 to 120 cps, a methoxy content of 19% to 24% by weight and a hydroxypropoxy content of 7% to 12% by weight which may be obtained from The Dow Chemical Company, Michigan, USA. This product meets the specifications for HPMC 2208 USP. Note that the particular METHOCEL ® K100LV Premium CR utilized in this example must have a hydroxypropoxy content of less than 9.0% by weight.
(c) METHOCEL ® K4M Premium CR is hydroxypropyl methylcellulose with a viscosity of 3,500 to 5,600 cps, a methoxy content of 19% to 24% by weight and a hydroxypropoxy content of 7% to 12% by weight which may be obtained from The Dow Chemical Company, Michigan, USA. This product meets the specification of HPMC 2208 USP.
(d) Added but not retained

### Example 4

### Preparation of Composition

Following a procedure similar to that described in Example 1, tablets of the following composition can be prepared.

TABLE 4

| | mg\Tablet | % of Tablet |
|---|---|---|
| Active ingredient (a) | 345.38 | 43.2 |
| Lactose NF | 89.31 | 11.1 |
| Microcrystalline Cellulose NF | 89.31 | 11.1 |
| Sodium citrate | 100.00 | 12.5 |
| METHOCEL ® K100LV Premium CR (b) | 120.00 | 15.0 |
| METHOCEL ® E4M Premium CR (c) | 40.00 | 5.0 |
| Purified water (d) | q.s. | — |
| Magnesium stearate NF | 16.00 | 2.0 |

(a) The active ingredient is 11-[4-[2-(2-hydroxyethoxy) ethyl]-1-piperazinyl]-dibenzo[b,f][1,4]thiazepine hemifumarate
(b) METHOCEL ® K100LV Premium CR is hydroxypropyl methylcellulose with a viscosity of 80 to 120 cps, a methoxy content of 19% to 24% by weight and a hydroxypropoxy content of 7% to 12% by weight which may be obtained from The Dow Chemical Company, Michigan, USA. This product meets the specifications for HPMC 2208 USP. Note that the particular METHOCEL ® K100LV Premium CR utilized in this example must have a hydroxypropoxy content of less than 9.0% by weight.
(c) METHOCEL ® E4M Premium CR is hydroxypropyl methylcellulose with a viscosity of 3,500 to 5,600 cps, a methoxy content of 28% to 30% by weight and a hydroxypropoxy content of 7% to 12% by weight which may be obtained from The Dow Chemical Company, Michigan, USA. This product meets the specifications for HPMC 2910 USP.
(d) Added but not retained

### Example 5

### Preparation of Composition

Following a procedure similar to that described in Example 1, tablets of the following composition can be prepared.

TABLE 5

| | mg\Tablet | % of Tablet |
|---|---|---|
| Active ingredient (a) | 345.38 | 43.2 |
| Lactose NF | 69.31 | 8.7 |
| Microcrystalline Cellulose NF | 69.31 | 8.7 |
| Sodium citrate | 100.00 | 12.5 |
| METHOCEL ® K100LV Premium CR (b) | 200.00 | 25.0 |
| Purified water (d) | q.s. | — |
| Magnesium stearate NF | 16.00 | 2.0 |

(a) The active ingredient is 11-[4-[2-(2-hydroxyethoxy)ethyl]-1-piperazinyl]-dibenzo[b,f][1,4]thiazepine hemifumarate
(b) METHOCEL ® K100LV Premium CR is hydroxypropyl methylcellulose with a viscosity of 80 to 120 cps, a methoxy content of 19% to 24% by weight and a hydroxypropoxy content of 7% to 12% by weight which may be obtained from The Dow Chemical Company, Michigan, USA. This product meets the specifications for HPMC 2208 USP. Note that the particular METHOCEL ® K100LV Premium CR utilized in this example must have a hydroxypropoxy content of less than 9.0% by weight.
(c) Added but not retained.

### Example 6

### Preparation of Composition

Following a procedure similar to that described in Example 1, tablets of the following composition can be prepared.

TABLE 6

| | mg\Tablet | % of Tablet |
|---|---|---|
| Active ingredient (a) | 345.38 | 43.2 |
| Povidone USP (b) | 40.00 | 5.0 |
| Microcrystalline Cellulose NF | 38.62 | 4.8 |
| Sodium citrate | 200.00 | 25.0 |
| METHOCEL ® E5OLV Premium (c) | 80.00 | 10.0 |
| METHOCEL ® E4M Premium CR (d) | 80.00 | 10.0 |
| Purified water (e) | q.s | — |
| Magnesium stearate NF | 16.00 | 2.0 |

(a) The active ingredient is 11-[4-[2-(2-hydroxyethoxy)ethyl]-1-piperazinyl]- dibenzo[b,f][1,4]thiazepine hemifumarate
(b) This reagent is a polyvinylpyrrolidone polymer having a K-value of 29-32 which may be obtained from ISP Technologies Inc., Wayne, New Jersey, USA, under the trademark PLASDONE ® K-29/32. This product meets the specifications for Povidone USP.
(c) METHOCEL ® E50LV Premium is hydroxypropyl methylcellulose with a viscosity of 40-60 cps, a methoxy content of 28% to 30% by weight and a hydroxypropoxy content of 7% to 12% by weight which may be obtained from The Dow Chemical Company, Michigan, USA. This product meets the specifications for HPMC 2910 USP. Note that the particular METHOCEL ® E50LV Premium utilized in this example must have a hydroxypropoxy content of less than 9.0% by weight.
(d) METHOCEL ® E4M Premium CR is hydroxypropyl methylcellulose with a viscosity of 3,500 to 5,600 cps, a methoxy content of 28% to 30% by weight and a hydroxypropoxy content of 7% to 12% by weight which may be obtained from The Dow Chemical Company, Michigan, USA. This product meets the specifications for HPMC 2910 USP.
(e) Added but not retained

### Example 7

### Preparation of Composition

Following a procedure similar to that described in Example 1, tablets of the following composition can be prepared.

TABLE 7

| | mg\Tablet | % of Tablet |
|---|---|---|
| Active ingredient (a) | 345.38 | 43.2 |
| Povidone USP (b) | 40.00 | 5.0 |
| Microcrystalline Cellulose NF | 38.62 | 4.8 |
| Sodium citrate | 200.00 | 25.0 |
| METHOCEL ® E5OLV Premium (c) | 80.00 | 10.0 |
| METHOCEL ® E4M Premium CR (d) | 80.00 | 10.0 |
| Purified water (e) | q.s | — |
| Magnesium stearate NF | 16.00 | 2.0 |

(a) The active ingredient is 11-[4-[2-(2-hydroxyethoxy)ethyl]-1-piperazinyl]-dibenzo[b,f][1,4]thiazepine hemifumarate.
(b) This reagent is a polyvinylpyrrolidone polymer having a K-value of 90 which may be obtained from ISP Technologies Inc., Wayne, New Jersey, USA, under the trademark PLASDONE ® K-90. This product meets the specifications for Povidone USP.
(c) METHOCEL ® E50LV Premium is hydroxypropyl methylcellulose with a viscosity of 40-60 cps, a methoxy content of 28% to 30% by weight and a hydroxypropoxy content of 7% to 12% by weight which may be obtained from The Dow Chemical Company, Michigan, USA. This product meets the specifications for HPMC 2910 USP. Note that the particular METHOCEL ® E50LV Premium utilized in this example must have a hydroxypropoxy content of less than 9.0% by weight.
(d) METHOCEL ® E4M Premium CR is hydroxypropyl methylcellulose with a viscosity of 3,500 to 5,600 cps, a methoxy content of 28% to 30% by weight and a hydroxypropoxy content of 7% to 12% by weight which may be obtained from The Dow Chemical Company, Michigan, USA. This product meets the specifications for HPMC 2910 USP.
(e) Added but not retained.

### Examples 8-10

### Preparation of Compositions

Following a procedure similar to that described in Example 1, tablets of the following compositions were prepared:

TABLE 8

| | Example 8 | | Example 9 | | Example 10 | |
|---|---|---|---|---|---|---|
| | mg/tablet-% | | mg/tablet-% | | mg/tablet-% | |
| | of tablet | | of tablet | | of tablet | |
| Active Ingredient(a) | 345.38 | 43.2 | 345.38 | 43.2 | 345.38 | 43.2 |
| Lactose NF | 109.31 | 13.7 | 69.31 | 8.7 | 49.31 | 6.2 |
| Microcrystalline | 109.31 | 13.7 | 69.31 | 8.7 | 49.31 | 6.2 |
| Cellulose NF | | | | | | |
| Sodium citrate | 100.00 | 12.5 | 100.00 | 12.5 | 100.00 | 12.5 |
| METHOCEL ® | 120.00 | 15.0 | 200.00 | 25.0 | 200.00 | 25.0 |
| K100LV Premium | | | | | | |
| CR(b) | | | | | | |
| METHOCEL ® K4M | — | — | — | — | 40.00 | 5.0 |
| Premium CR(c) | | | | | | |
| Purified water(d) | q.s. | — | q.s. | — | q.s. | — |
| Magnesium stearate | 16.00 | 2.0 | 16.00 | 2.0 | 16.00 | 2.0 |
| NF | | | | | | |
| (a)The active ingredient is 11-[4-[2-(2-hydroxyethoxy)ethyl]-1-piperazinyl]-dibenzo[b,f][1,4]thiazepine hemifumarate | | | | | | |
| (b)METHOCEL ® K100LV Premium CR is hydroxypropyl methylcellulose with a viscosity of 80 to 120 cps, a methoxy content of 19% to 24% by weight and a hydroxypropoxy content of 7% to 12% by weight which may be obtained from The Dow Chemical Company, Michigan, USA. This product meets the specifications for HPMC 2208 USP. Note that the particular METHOCEL ® K100LV Premium CR utilized in this example had a viscosity of 90 cps, a methoxy content of 22.7% by weight and a | | | | | | |
| # hydroxypropoxy content of 8.5% by weight. | | | | | | |
| (c)METHOCEL ® K4M Premium CR is hydroxypropyl methylcellulose with a viscosity of 3,500 to 5,600 cps, a methoxy content of 19% to 24% by weight and a hydroxypropoxy content of 7% to 12% by weight, which may be obtained from The Dow Chemical Company, Michigan, USA. This product meets the specification of HPMC 2208 USP. Note that the particular METHOCEL ® K4M Premium CR utilized in this example had a viscosity of 4105 cps, a methoxy content of 22.3% by weight and a | | | | | | |
| # hydroxypropoxy content of 9.7% by weight. | | | | | | |

(d)Added but not retained.

### Example 11

### Preparation of Composition

Following a procedure similar to that described in Example 1, tablets of the following composition were prepared:

TABLE 9

| | mg/Tablet | % of Tablet |
|---|---|---|
| Active ingredient (a) | 345.38 | 43.2 |
| Povidone USP (b) | 80.00 | 10.00 |
| Sodium citrate USP | 100.00 | 12.5 |
| Microcrystalline cellulose NF | 138.62 | 17.3 |
| METHOCEL ® E4M Premium CR (c) | 120.00 | 15.0 |
| Purified water (d) | q.s. | — |
| Magnesium Stearate NF | 16.0 | 2.0 |
| (a) The active ingredient is 11-[4-[2-(2-hydroxyethoxy)ethyl]-1-piperazinyl]-dibenzo[b,f][1,4]thiazepine hemifumarate | | |
| (b) This reagent is a polyvinylpyrrolidone polymer having a K-value of 90 which may be obtained from ISP Technologies Inc., Wayne, New Jersey, USA, under the trademark PLASDONE ® K-90. This product meets the specifications for Povidone USP. | | |
| (c) METHOCEL ® E4M Premium CR is hydroxypropyl methylcellulose with a viscosity of 3,500 to 5,600 cps, a methoxy content of 28% to 30% by weight and a hydroxypropoxy content of 7% to 12% by weight which may be obtained from The Dow Chemical Company, Michigan, USA. This product meets the specifications for HPMC 2910 USP. Note that the particular METHOCEL ® E4M Premium CR utilized in this example had a viscosity of 4364 cps, a methoxy content of 28.5% by | | |
| # weight and a hydroxypropoxy content of 7.8% by weight. | | |

(d) Added but not retained.

### Example 12

### Preparation of Composition

Sustained release formulations of quetiapine fumarate are set forth in Tables 10-12 below. They are prepared by:

1) Mixing quetiapine fumarate, lactose, microcrystalline cellulose, HPMC (name changed to hypromellose), and sodium citrate (e.g., in a high shear granulator) until content uniformity is achieved (e.g., 600 L Fielder for about 10 minutes);

2) Charging purified water (e.g., 37% by weight of the tablet) onto the powder in the granulator (eg., spray nozzle) 5-6 minutes] to form a granulate;

3) Drying the granulate (fluid bed ;) (e.g., to a moisture content of <or equal to 3% Loss on drying);

4) Reducing the particle of the granulate to achieve a suitable flow for compression (e.g., Carr index that does not exceed 30 (e.g., 20); (using e.g., 0.05 to 0.109 inch mill screen);

5) Blending the granulate with magnesium stearate for a time sufficient to prevent substantial tablet punch filming (e.g., 3 minutes in a V blender; ⅔ full).

The resulting formulation of step 5 is compressed to form a tablet having a hardness of greater than 16 kiloponds (particularly about 28 kp) and a friability of less than 1%).

The tablets may further be coated by mixing all the (coating) ingredients in water until dissolved and spray the resulting mixture spray onto the tablet (for example in perforated pan coater) until a uniform coat is achieved (e.g., a target of 2.5% percent by weight).

TABLE 10
Formulations and Tablets for Quetiapine Fumarate

| | Seroquel | Weight Percent |
|---|---|---|
| | SR | (%) of |
| Ingredients | 400 mg | Formulation |
| Tablet materials | | |
| Quetiapine fumarate | 460.50 | 52.9 |
| Lactose mono- | 15.50 | 1.8 |
| hydrate | | |
| Microcrystalline | 15.60 | 1.8 |
| cellulose | | |
| Sodium citrate | 100.00 | 11.5 |
| dihydrate | | |
| Hypromellose 2208 | | 27 |
| 100 cP | | |
| Hypromellose 2208 | | 3 |
| 4000 cP | | |
| Magnesium stearate | 17.40 | 2.0 |
| Purified water | qs | |
| Total Tablet Weight | 870.00 | |
| Coating materials | | |
| Hypromellose 2910^{d} | 13.63 | |
| Polyethylene glycol | 2.73 | |
| 400 NF | | |
| Titanium dioxide^{e} | 5.45 | |
| Ferric oxide, yellow^{f} | | |
| Ferric oxide, red^{g} | | |
| Purified water | 123.6 | |
| Total Coating Weight | 21.8 | |

TABLE 11
Formulations and Tablets for
Quetiapine Fumarate

| | | Weight |
|---|---|---|
| | | Percent (%) |
| | Seroquel SR | of |
| Ingredients | 200 mg | Formulation |
| Tablet materials | | |
| Quetiapine fumarate | 230.26 | 38.4 |
| Lactose mono- | 52.87 | 8.9 |
| hydrate | | |
| Microcrystalline | 52.87 | 8.9 |
| cellulose | | |
| Sodium citrate | 75.00 | 12.5 |
| dihydrate | | |
| Hypromellose 2208 | | 23 |
| 100 cP | | |
| Hypromellose 2208 | | 7 |
| 4000 cP | | |
| Magnesium stearate | 9.00 | 1.5 |
| Purified water | qs | |
| Total Tablet Weight | 600.00 | |
| Coating materials | | |
| Hypromellose 2910^{d} | 8.82 | |
| Polyethylene glycol | 2.65 | |
| 400 NF | | |
| Titanium dioxide^{e} | 3.27 | |
| Ferric oxide, yellow^{f} | 0.26 | |
| Ferric oxide, red^{g} | | |
| Purified water | 135.0 | |
| Total Coating | 15.0 | |
| Weight | | |

TABLE 12
Formulations and Tablets for
Quetiapine Fumarate

| | | Weight |
|---|---|---|
| | | Percent (%) |
| | Seroquel SR | of |
| Ingredients | 50 mg | Formulation |
| Tablet materials | | |
| Quetiapine fumarate | 57.56 | 11.6 |
| Lactose mono- | 125.72 | 25.1 |
| hydrate | | |
| Microcrystalline | 125.72 | 25.1 |
| cellulose | | |
| Sodium citrate | 36.00 | 7.2 |
| dihydrate | | |
| Hypromellose 2208 | 150.00 | 23 |
| 100 cP | | |
| Hypromellose 2208 | | 7 |
| 4000 cP | | |
| Magnesium stearate | 5.00 | 1 |
| Purified water | qs | |
| Total Tablet Weight | 500.00 | |
| Coating materials | | |
| Hypromellose 2910^{d} | 7.35 | |
| Polyethylene glycol | 2.21 | |
| 400 NF | | |
| Titanium dioxide^{e} | 2.72 | |
| Ferric oxide, yellow^{f} | 0.11 | |
| Ferric oxide, red^{g} | 0.11 | |
| Purified water | 112.50 | |
| Total Coating | 12.5 | |
| Weight | | |

The hydroxypropyl content of the 100 cP Hypromellose 2208 is in the range of 10.5 to 11.2 % by wieght and the hydroxypropyl content of the 4000 cP Hypromellose 2208 is in the range of 10.8 to 11.9% by weight and is determined for example using an NMR method 5 described below.

Determination of Hydroxypropyl (HP) Content of Hydroxypropyl Methylcellulose (Hypromellose) by Nuclear Magnetic Resonance 3.5 to about 4.5 mg of hypromellose is dissolved in a solvent, which is 99.96% D₂O.

the hypromellose is heated at about 105° C. for about 30 minutes prior to dissolving in the solvent.
the hypromellose is heated at about 80° C. for about 15 minutes after dissolving in the solvent.

NMR particulars
the nuclear magnetic resonance spectrometer comprises a ¹H{x} inverse detection probe.
the temperature is about 353K.
the pulse is about 45°.
the spectrum width is about −2.5 to 13.5 ppm.
the pulse repetition is about 15 seconds.
the exponential line broadening is about 1.0 Hz.
the spectrum is referenced to residual DMSO peak at 2.70 ppm.
the baseline of the nuclear magnetic resonance spectrum is corrected.
the number of scans is selected such that the signal:noise at 200 Hz for the peak at 1.2 ppm is greater than 500.
the number of time domain data points is about 65,000.
the number of processed data points is about 250,000.

b. NMR spectrum is phased so that the peaks at 4.5 ppm and 1.2 ppm are symmetric.

c. The following regions are integrated:
i) Region 1: 4.96-4.31, which is Area A;
ii) Region 2: 4.08-2.95, which is Area B; and
iii) Region 3: 1.47-0.92, which is Area C; and

d. Calculate the hydroxypropoxy content (weight % HP) as:
Weight % HP={(75×MoleHP)/[162+(58×MoleHP)+(14×MoleMeO)]}×100,
wherein:
i) MoleHP=C/(3>A);
ii) MoleMeO=[B—C-(6—A)]/(3×A); and

MeO is methoxy.

Exemplary Procedure for NMR Analysis:

a) heating a 3.5 to 4.5 mg sample of hypromellose at about 105° C. for about 30 minutes;

b) dissolving the 3.5 to 4.5 mg sample of hypromellose in 99.96% D₂O;

c) heating the dissolved hypromellose at about 80° C. for about 10 minutes;

d) analyzing the dissolved hypromellose by nuclear magnetic resonance whereby:
i) the nuclear magnetic resonance spectrometer comprises a ¹H{x} inverse detection probe;
ii) the temperature is about 353K;
iii) the pulse is about 45°;
iv) the spectrum width is about −3.5 to 13.5 ppm;
v) the pulse repetition is about 15 seconds;
vi) the exponential line broadening is about 1.0 Hz;
vii) the number of scans is selected such that the signal:noise at 200 Hz for the peak at 1.2 ppm is greater than 500;
viii) the number of time domain data points is about 65,000; and
ix) the number of processed data points is about 250,000;
e) phasing the nuclear magnetic resonance spectrum so that the peaks at 4.5 ppm and 1.2 ppm are symmetric;
g) referencing the spectrum to residual DMSO peak at 2.70 ppm;
g) correcting the baseline of the nuclear magnetic resonance spectrum;
h) integrating the following regions:
   i) Region 1: 4.96-4.31, which is Area A;
   ii) Region 2: 4.31-4.08;
   iii) Region 3: 4.08-2.95, which is Area B;
   iv) Region 4: 2.95-2.45; and
   v) Region 5: 1.47-0.92, which is Area C; and
i) calculating the hydroxypropoxy content (weight % HP) as: Weight % HP={(75×MoleHP)/[162+(58×MoleHP)+(14×MoleMeO)]}×100, wherein:
   i) MoleHP=C/(3×A); and
   ii) MoleMeO=[B—C-(6×A)]/(3×A).

### Example 13

### Preparation of Immediate Release Composition

Following a procedure similar to that described in Example 1, tablets of the following composition were prepared:

TABLE 13

| | Mg/Tablet |
|---|---|
| CORE | |
| Active ingredient (a) | 115.13 |
| Povidone USP (b) | 8.33 |
| Dicalcium phosphate dihydrate USP | 10.00 |
| Microcrystalline cellulose NF | 32.88 |
| Sodium starch glycolate NF | 8.33 |
| Lactose NF | 22.33 |
| Magnesium stearate NF | 3.00 |
| Purified water (c) | q.s. |
| COATING | |
| Hydroxypropyl methylcellulose 2910 USP (d) | 5.00 |
| Polyethylene glycol 400 NF | 1.00 |
| Yellow ferric oxide NF | 0.15 |
| Titanium dioxide USP | 1.85 |

(a) The active ingredient is 11-[4-[2-(2-hydroxyethoxy)ethyl]-1-piperazinyl]dibenzo[b,f][1,4]thiazepine hemifumarate.
(b) This reagent is a polyvinylpyrrolidone polymer having a K-value of 29-32 which may be obtained from ISP Technologies Inc., Wayne, New Jersey, USA, under the trademark PLASDONE ® K-29/32. This product meets the specification for Povidone USP.
(c) Added but not retained.
(d) The hydroxypropyl methylcellulose utilized in this example was PHARMACOAT ® 606 which may be obtained from Shin-Etsu, Ltd., Japan and has a viscosity in the range of 4.5 to 8.0 cps, a methoxy content of 28% to 30% by weight and a hydroxypropoxy content of 7% to 12% by weight.

The above described immediate release composition is prepared by the following process. The active ingredient, povidone, dicalcium phosphate dihydrate, and portions of the microcrystalline cellulose and sodium starch glycolate are mixed in a mixer-granulator (for example, a Littleford MGT) for approximately 5 minutes. Purified water is added while mixing until a suitable mass is obtained. The wet granules are passed through a cone mill fitted with an appropriate screen (e.g. 6.35 mm) and then dried in a fluidized bed dryer set at an inlet temperature of approximately 65° C. to a loss on drying level of less than 2.5% w/w. The dried granules are then passed through a suitable mill fitted with an appropriate screen (e.g. #20 mesh in a hammer mill). The granulation is combined in a blender (e.g. V-blender) with lactose and the remainder of the microcrystalline cellulose and sodium starch glycolate and is blended for approximately 5 minutes. The magnesium stearate is passed through a suitable mill fitted with an appropriate screen (e.g. 40 mesh) and then added to the dry granulated material and blended for approximately 3 minutes. The blended mixture is then compressed into tablets using conventional rotary compression equipment. The tablets are then film coated using conventional drum coating equipment with an aqueous suspension of the film coating constituents (i.e. hydroxypropyl methylcellulose, polyethylene glycol 400, yellow ferric oxide and titanium dioxide) at an inlet temperature of approximately 80° C.

### Example 14

### Monotherapy in the Maintenance Treatment of MDD

This study is a double-blind, placebo-controlled evaluation of the efficacy of quetiapine as monotherapy for up to 52 weeks of maintenance treatment of MDD using 50, 150, and 300 mg/day dosing regimen. The study comprises four periods: an enrollment period of up to 28 days; an Open-Label Run-In period of 2 to 8 weeks, an Open-Label Stabilization Treatment (OLST) period of 4 months, and a Randomized Treatment period of up to 52 weeks (treatment with a sustained release form of quetiapine or placebo). Patient eligibility criteria includes male or female patients 18 to 65 years old, with a documented clinical diagnosis of MDD together with an acute depressed episode confirmed by Mini-Intemational Neuropsychiatric Interview (MINI) and meeting the DSM-IV of either:

Criteria 296.2× MDD, Single Episode; or

Criteria 296.3× MDD, Recurrent

The patient must have a current episode of depression that is less than 12 months and at least 4 weeks in duration prior to enrollment. The patients should also have a HAM-D score≧20 at enrollment to be eligible for the study.

Treatment Groups

All patients who complete this study will receive a sustained release form of quetiapine for a minimum of 18 and a maximum of 76 weeks. In order to assess the effect of a sustained release form of quetiapine during the maintenance period, it is necessary to randomize a proportion of the patients to placebo. During the Randomized Treatment Period half of the patients will receive a sustained release form of quetiapine, and thus receive active treatment for an even longer duration. The patients who are randomized to receive placebo will be assessed frequently according to the study plan. If they meet criteria for a depressed event, they will be discontinued from the study and may receive treatment as usual according to the investigator's judgment.

Once enrolled and all eligibility criteria are met, the patients enter the Open-Label Run-In Period that can last up to 8 weeks followed by the OLST Period. During the OLST Period, patients will be treated with open-label sustained release form of quetiapine for 4 months. During the Randomized Treatment Period the patient is randomized to either a sustained release form of quetiapine or placebo at the same dose as taken at the last visit of the OLST Period for a 52-week treatment period. Entry criteria for each study period are provided in the Table below.

TABLE 14
Criteria to Enter Study Periods

| | | Entry to OLST | During | Randomization |
|---|---|---|---|---|
| Entry | Enrolment | At any OLT visit | OLST | Day of |
| Criteria^{a}: | Entry | between 2-8 wks | 0-16 wks | randomization |
| HAMD | | | | |
| 17-item | ≧20 | | | |
| Item 1 | ≧2 | | | |
| MADRS | | ≦12 | N/A | ≦12 |
| CGI-S | | ≦3 | ≦4 | N/A |

OLT = Open-label Treatment
OLST = Open-label Stabilisation Treatment

Allowable visit windows are provided in Table 15.

TABLE 15
Visit Windows

| Visit | | Open-Label | Randomized Treatment |
|---|---|---|---|
| Windows | Visit Interval | Treatment Period | Period |
| ±2 days | Weekly | Visits 3, 4 | Visits 18, 19 |
| ±3 days | Bi-weekly | Visits 5, 6, 7 | Visit 20 |
| ±7 days | Every 4 weeks | Visits 8, 9, 10, 11 | Visits 21-32 |

Study medication will be administered once daily in the evening. Treatment 15 discontinuation symptoms will be collected for 14 days following the last dose of open-label treatment in randomized patients. Discontinuation Emergent Signs and Symptoms (DESS) will not be assessed in patients who completed or were discontinued during the open-label treatment and did not enter randomization.

This study can include: (1) evaluation of the efficacy of a sustained release form of quetiapine compared to placebo in maintaining improvement of depressive symptoms in patients with MDD during long-term treatment, as assessed by: (a) the change from randomization to each assessment in the MADRS total score; (b) the incidence of relapse which is defined as a depressed event according to the criteria defined above; (c) the change from randomization to each assessment in the Clinical Global Impression—Severity of Illness (CGI-S); (2) evaluation of the efficacy of a sustained release form of quetiapine compared to placebo in treating anxiety symptoms in patients with MDD during long-term treatment, as assessed by: (a) the change from randomization to each assessment in Hamilton Rating Scale for Anxiety (HAM-A); (b) the change from randomization in the HAM-A psychic anxiety factors (Anxious Mood, Tension, Fears, Insomnia, Intellect, Depressed Mood, Behavior at Interview); (c) The change from randomization in the HAM-A somatic anxiety factors (Somatic Muscular, Somatic Sensory, Cardiovascular, Respiratory, Gastrointestinal, Genitourinary, Autonomic); (3) evaluation of the effect of a sustained release form of quetiapine on the quality of sleep in patients with MDD, compared to placebo during long-term treatment as measured by the change from randomization in Pittsburgh Sleep Quality Index (PSQI) global score; (4) evaluation of the effect of a sustained release form of quetiapine on suicidal ideation in patients with MDD compared to placebo during long-term treatment, as assessed by the change from randomization in the MADRS item 10, suicidal thought; (5) evaluation of the effect of a sustained release form of quetiapine on quality of life of patients with MDD compared to placebo during long-term treatment, as assessed by the change from randomization in Quality of Life Enjoyment and Satisfaction Questionnaire (Q-les-Q) total score (item 1-14); (6) evaluation of the effect of a sustained release form of quetiapine on functional disability in patients with MDD compared to placebo during long-term treatment, as assessed by the change from randomization in the Sheehan Disability Scale (SDS) total score; and/or (7) evaluation if a sustained release form of quetiapine is safe and well-tolerated in the long-term treatment of patients with MDD and to evaluate if a sustained release form of quetiapine is as safe and well-tolerated as placebo in the long-term treatment of patients with MDD, as assessed by (a) the change from normal to Clinically Important in physical examinations including eye exams, laboratory values (including glucose/lipids), vital signs and electrocardiograms (ECGs), (b) the incidence of Adverse Events (AE); (c) adverse events related to sexual dysfunction, nausea, vomiting, and extrapyramidal symptoms including akathisia; (d) The change in Abnormal Involuntary Movement Scale (AIMS), Simpson-Angus Scale (SAS) and Barnes Akathisia Rating Scale (BARS) over the course of treatment; (e) MADRS item 10 score≧4 at any time after randomization or AE of suicidality/suicidal ideation/suicide attempts/suicide completion; (f) Incidence of suicidality using Columbia University classification; (g) total withdrawals due to adverse events; (h) serious discontinuation symptoms assessed by DESS scale; (i) the incidence of AEs related to somnolence, the severity of somnolence AEs, time to onset of somnolence AEs, and withdrawal from study due to adverse event of somnolence; (j) the change in weight and waist circumference over the course of treatment; and/or (k) the proportion of patients with a ≧7% increase in weight over the course of treatment.

Open-Label Run-In Treatment Period (2-8 Weeks)

During the Open-Label Run-In Period, patients will be treated with open-label sustained release form of quetiapine for 2 to 8 weeks. All patients will titrate to a 150 mg/day dose from Day 1 to Day 6 (Table 13). The starting dose of a sustained release form of quetiapine will be 50 mg/day during Days 1-3. The dose of a sustained release form of quetiapine will then be up-titrated to 150 mg/day during Days 4-6. After Day 6, the dosage of a sustained release form of quetiapine can be increased to 300 mg/day or decreased to 50 mg/day based upon the clinical judgment of the investigator to maximize efficacy and tolerability. Patients will return for an unscheduled visit if dose adjustment is required. If depressive symptoms of the patient are not adequately controlled, the investigator should consider adjusting the sustained release form of quetiapine dose to a maximum of 300 mg/day prior to discontinuation of the patient. Doses are 50, 150, and 300 mg/day.

TABLE 16
Titration of a Sustained Release Form of Quetiapine

| Days | Sustained Release Form of Quetiapine dose |
|---|---|
| Days 1-3 | 50 mg/day |
| Days 4-6 | 150 mg/day |

At any visit under this period, if the patient meets the OLST criteria (MADRS score=12 and CGI-S score =3), he/she can begin the OLST Period. Patients who do not meet the criterion for entering the OLST Period (MADRS score=12) during the Open Label Run-in Treatment Period are discontinued from the study.
OLST Period (4 Months)

During the OLST Period, patients will be treated with open-label sustained release form of quetiapine for 4 months. One purpose of the Open-Label Stabilization Period is to achieve stabilization after acute treatment of depression before randomization to double-blind treatment.

Patients will start on the same dose of a sustained release form of quetiapine as taken at the last visit of the Open-label Run-In Treatment Period. The prescribed sustained release dosage should be adjusted to 50, 150 or 300 mg/day once daily to maximize efficacy and tolerability. If depressive symptoms of the patient are not adequately controlled, the investigator should consider adjusting the sustained release form of quetiapine dose to a maximum of 300 mg/day prior to discontinuation of the patient. Doses are 50, 150, and 300 mg/day.

Visits will occur every 4 weeks. During this period the MADRS score is allowed to increase up to 14.

Treatment with open-label sustained release form of quetiapine will continue until the patient has completed 4 months of open-label treatment. After this period the patient should have a MADRS score=12 to be eligible for randomization. If this criteria is not met at the 4-month visit, the patient may return to the clinic for up to 3 more visits (up to 6 weeks after end of Open-Label Stabilization Period) to meet the criteria. Patients who do not meet the criteria for entering the Randomized Treatment Period are discontinued from the study.

Recruitment of patients to the OLST Period will cease when it is estimated that a sufficient number of patients have been recruited to provide a total of at least 88 depressed events.

Randomized Treatment Period (Up to 52 Weeks)

Patients who meet all the inclusion criteria for randomization and none of the exclusion criteria for randomization will be randomized (at any of the visits 11-14) in a blinded fashion to a sustained release form of quetiapine or placebo at the same dose as taken at the last visit of the OLST Period. The dosage can be adjusted to 50, 150, or 300 mg/day, as clinically indicated during the study.

Patients will continue in the Randomized Treatment Period for up to 52 weeks or until they meet any of the criteria for a depressed event (defined above), or until the study is terminated. Once relapse of a depressed event occurs, the patient must be discontinued from the study. Patients may also be discontinued from study treatment and assessments due to lack of efficacy, AE, patient lost to follow-up, protocol non-compliance, informed consent withdrawn.

Patients who reach a MADRS total score of =18 will be required to return to the study site the following week to repeat the MADRS assessment. Patients must have a MADRS total score of =18 at both assessments to be qualified as a depressed event and discontinued from the study. If the patient discontinues from the study after first assessment of MADRS total score of =18, then the patient will also be qualified as having a depressed event.

The study physician must document a CGI-S score of =5 (markedly ill) at a study visit or by a phone call interview within 1 week of a missed study visit in order for the possible event to be qualified as a depressed event.

Patients who are prescribed a medication by a physician to treat MDD will qualify as a depressed event. Additionally, patients who self-medicate with exclusionary medications to treat MDD for 1 week or greater will be qualified as a depressed event and be discontinued. Patients who meet the criteria for a depressed event are discontinued from the study.

Rating Scales and Endpoints

The primary efficacy variable is the time to a depressed event from randomization. This is a clinically relevant endpoint and has been frequently used in maintenance studies. The MADRS is a standardized, well-validated measure of depressive symptoms that is sensitive to treatment effects in depressed outpatients. The threshold value for classifying patients in remission (MADRS=12) has been chosen to detect patients who have resolution of symptoms. This cut-off level has been widely used in other studies.

Enrollment

Exclusion Criteria

Any of the following is regarded as a criterion for exclusion from the study: (1) patients with a DSM-IV Axis I disorder other than MDD within 6 months of enrolment; (2) patients whose current episode of depression exceeds 12 months or is less than 4 weeks from enrolment; (3) history of inadequate response to an adequate treatment (6 weeks) with 2 or more classes of antidepressants during current depressive episode; (4) patients who, in the investigator's judgment, pose a current serious suicidal or homicidal risk, have a HAM-D item 3 score of 3 or greater, or have made a suicide attempt within the past 6 months; and (5) known lack of response to quetiapine in the treatment of depression in a dosage of at least 150 mg/day for 4 weeks, as judged by the investigator.

TABLE 17
Prohibited pre-study medications and treatments

| Medication or Treatment | Time period |
|---|---|
| Antipsychotic medications | 7 days prior to open-label treatment |
| Mood stabilisers and | 7 days prior to open-label treatment |
| anticonvulsants | (except for carbamazepine, 14 days; |
| | see potent cytochrome P450 |
| | 3A4.inducer) |
| Antidepressant medication | 7 days prior to open-label treatment, |
| | except fluoxetine within 28 days |
| | before open-label treatment |
| MAO Inhibitors | 14 days prior to open-label treatment |
| Benzodiazepines | 7 days prior to open-label treatment |
| Anxiolytics | 7 days prior to open-label treatment |
| Hypnotics | 7 days prior to open-label treatment |
| | unless used regularly for the treatment |
| | of insomnia |
| Depot antipsychotic injection | Within 2 dosing intervals prior to |
| | open-label treatment |
| Cytochrome P450 (CYP) 3A4 | 14 days prior to open-label treatment |
| inhibitors or inducers (potent) | |
| Electroconvulsive therapy (ECT) | 28 days before enrolment |

Patient-Reported Outcomes (PROs)

The methods for collecting Patient Reported Outcomes (PRO) data are presented below. The Q-les-Q, PSQI and SDS will be completed by the patient at Day 0 (Visit 2) of the Open-Label Treatment Period, on Day of randomization, and at Weeks 4, 16, 28, 40 and 52 (Visits 20, 23, 26, 29, and 32) of the Randomized Treatment Period.

Quality of Life Enjoyment and Satisfaction Questionnaire (Q-les-Q)

Methods of assessment: The Q-les-Q will be completed at scheduled visits during the trial by each patient. The instrument has been developed to measure differences in degree of enjoyment and satisfaction. The short form used in this trial has 16 items. It has the same content as the last section (General Activities) of the regular version of the Q-les-Q. The first 14 items will be used to derive a total score, and the remaining 2 are single items, measuring satisfaction with medication and overall life satisfaction, respectively. Higher scores indicate better health-related quality of life. The instrument is sensitive to change over time following treatment. It has been found to have high internal consistency, test-retest reliability, and concurrent validity in patients with MDD and GAD.

Derivation or calculation of variable: The Q-les-Q total score is derived by summing item scores 1-14, and expressing them as a percentage of the maximum possible score (ranging from 0 to 100). For all Q-les-Q variables, the change from randomization to each assessment will be calculated as the visit score minus the score at randomization

PSQI

Method of assessment: The PSQI will be completed at scheduled visits during the trial by each patient. The 24-item scale is a reliable, valid and standardized measure of sleep quality. It covers several dimensions that impact sleep quality, such as subjective sleep latency, sleep duration, habitual sleep efficiency, sleep disturbances, use of sleep medications, and daytime dysfunction. Of the 24 items, 19 are self-rated, and a bed partner or roommate, if available, rates 5 items. Only the 19 self-rated items will be used in this trial. A global score is available. Higher scores indicate more severe difficulties in sleep quality.

Derivation or calculation of variable: The 19 self-rated time scores will be combined to form 7 component scores (subjective sleep quality, sleep latency, sleep duration, habitual sleep efficiency, sleep disturbances, use of sleep medications, and daytime dysfunction). Each component score is scored on a 0 to 3 scale. The PSQI is calculated as the sum of the 7 component scores. The change from randomization at each assessment will be calculated as the visit score minus the randomization score.

SDS

Method of assessment: The SDS will be completed at scheduled visits during the trial by each patient. The SDS is made up of 5 items that measure the extent a patient is impaired by the disease. It evaluates 3 inter-correlated domains (school/work, social life, and family life/home responsibilities) and measures the number of unproductive or under-productive days. Each of the three domains is rated from 0-10 (no impairment to most severe impairment) with evaluation of not at all (0), mild (1-3), moderate (4-6), marked (7-9), and extreme (10) disability. A score of 30 indicates most severe impairment.

Derivation or calculation of variable: The SDS total score will be calculated as the sum of the first 3 items (school/work, social life, and family life/home responsibilities). The change from randomization at each assessment and final assessment in the SDS total score, number of unproductive days and under-productive days will be calculated for each assessment

Efficacy and Pharmacodynamic Measurement and Variables

The efficacy variables of this study relate to the study objectives. A primary objective is to evaluate the efficacy of a sustained release form of quetiapine compared to placebo in increasing time to relapse of depression. Primary variable include: time from randomization to depressed event—a depressed event is defined as one of the following: a) initiation of pharmacological treatment by the Investigator, other than the allowed hypnotics, to treat depressive symptoms, b) initiation of pharmacological treatment by the patient for at least one week, other than the allowed hypnotics, to treat depressive symptoms, c) hospitalisation for depressive symptoms, d) MADRS score≧18 at 2 consecutive assessments or at the final assessment if the patient discontinues, e) CGI-S score of =5 (“markedly ill”), and f) suicide attempt. A supportive variable is time from randomization to all-cause discontinuation.

A secondary objective is to evaluate the efficacy of a sustained release form of quetiapine compared to placebo in maintaining improvement of depressive symptoms in patients with MDD during long-term treatment. Secondary variables include, for example, incidence of depressed events according to the criteria for primary variable; MADRS total score; and CGI-S.

Another objective is to evaluate the efficacy of a sustained release form of quetiapine compared to placebo in treating anxiety symptoms in patients with MDD during long-term treatment. Variables include, for example, HAM-A total score; HAM-A psychic anxiety factors score; and HAM-A somatic anxiety factors score.

Another objective is to evaluate the effect of a sustained release form of quetiapine compared to placebo on the quality of sleep in patients with MDD during long-term treatment. Variables include, for example, PSQI global score.

Another objective is to evaluate the efficacy of a sustained release form of quetiapine compared to placebo in treating suicidal ideation in patients with MDD during long-term treatment. Variables include, for example, MADRS item 10.

Another objective is to evaluate the effect of a sustained release form of quetiapine compared to placebo on the quality of life of patients with MDD during long-term treatment. Variables include, for example, Q-les-Q total score and Q-les-Q item 16.

Another objective is to evaluate the effect of a sustained release form of quetiapine compared to placebo on functional disability in patients with MDD during long-term treatment. Variables include, for example, SDS total score.

Montgomery-Asberg Depression Rating Scale (MADRS)

Methods of assessment: The MADRS is a 10-item scale for the evaluation of depressive symptoms. All MADRS assessments should evaluate the patient's symptoms during the past week. Each MADRS item is rated on a 0 to 6 scale. Higher MADRS scores indicate higher levels of depressive symptoms.

Calculation or derivation of outcome variable: The MADRS total score will be calculated as the sum of the 10 individual item scores and the total score ranges from 0-60. The change from baseline value to each assessment will be derived for the MADRS total score. Change from baseline to each assessment will be calculated for total MADRS score as the visit score minus the baseline score.

Hamilton Rating Scale—Anxiety (HAM-A)

Methods of assessment: The HAM-A is a 14-item clinician-administered scale for the evaluation of anxiety symptoms. The HAM-A will be administered by use of the Structured Interview Guide for the Hamilton Anxiety Rating Scale (SIGH-A) at each scheduled visit. All HAM-A assessments should evaluate the patient's symptoms during the past week. Each HAM-A item is rated on a 0 to 4 scale. Higher HAM-A scores indicate higher levels of anxiety.

Derivation or calculation of outcome variable: The HAM-A total score will be calculated as the sum of the 14 individual item scores. The HAM-A psychic anxiety factor score will be calculated as the sum of the following 7 items: anxious mood, tension, fears, insomnia, intellectual changes, depressed mood, and behavior at the interview. The HAM-A somatic anxiety factor score will be calculated as the sum of the following 7 items: somatic muscular, somatic sensory, cardiovascular system, respiratory system, gastrointestinal system, genitourinary system, and autonomic system.

The change from baseline to each assessment will be calculated for the HAM-A total score, HAMA-A psychic anxiety factor score, and HAM-A somatic anxiety factor score as the visit score minus the baseline score.

Clinical Global Impressions

Methods of Assessments: The Clinical Global Impressions (CGI) is a 3-part, clinician-administered scale that assesses global illness severity and change. For the purposes of this study, only the first part of the scale, CGI-severity, will be used. Each CGI-S item is scored on a scale from 1 to 7. A CGI-S score of 1 indicates that a patient is “Normal, not ill” and a score of 7 indicates that a patient is “Among the most extremely ill patients”. The CGI-S item score should be evaluated based on the prior week or visit. The CGI is administered at various times during the course of the study to assess patient progress. Higher CGI-S scores indicate greater illness severity.

Derivation or calculation of outcome variable: The change from baseline value to each assessment will be calculated for the CGI-S as the visit score minus the baseline score.

Safety Measurements and Variables

Safety will be evaluated in terms of adverse events (AE, including SAE), discontinuations due to AE, clinical laboratory analyses (including glucose, lipids and absolute neutrophil counts), vital signs, weight (including the percentage of patients with =7% increase from randomization weight), waist circumference, BMI, ECG changes, physical examination, extrapyramidal symptoms (BARS, SAS and AIMS), incidence of adverse events related to somnolence, severity and time of somnolence event, withdrawal due to somnolence. In addition, discontinuation symptoms will be assessed using the DESS scale.

Adverse Events (AEs)

An adverse event is the development of an undesirable medical condition or the deterioration of a pre-existing medical condition following or during exposure to a pharmaceutical product, whether or not considered causally related to the product. An undesirable medical condition can be symptoms (e.g., nausea, chest pain), signs (e.g., tachycardia, enlarged liver) or the abnormal results of an investigation (e.g., laboratory findings, electrocardiogram). In clinical studies, an AE can include an undesirable medical condition occurring at any time, including run-in or washout periods, even if no study treatment has been administered.

Serious Adverse Events (SAEs)

A serious adverse event is an AE occurring during any study period (i.e., run-in, treatment, washout, follow-up), and at any dose of the investigational product, comparator or placebo, that fulfills one or more of the following criteria: results in death, is immediately life-threatening, requires in-patient hospitalization or prolongation of existing hospitalization, results in persistent or significant disability or incapacity, is a congenital abnormality or birth defect, is an important medical event that may jeopardize the patient or may require medical intervention to prevent one of the above listed outcomes.

Other Significant Adverse Events (OAEs)

OAEs will be identified during the evaluation of safety data. Significant adverse events of particular clinical importance, other than SAEs and those AEs leading to discontinuation of the patient from study treatment, will be classified as OAEs. Examples of these are marked hematological and other laboratory abnormalities, and certain events that lead to intervention (other than those already classified as serious), dose reduction or significant additional treatment.

Rating Scales/Patient Reported Outcomes

Adverse event of Special Interest—Suicidality

All adverse events of suicidality will be carefully monitored. These include events of suicide attempts, suicide ideation, completed suicides and suicidal behavior. The last category includes behavioral AEs or SAEs in which the investigator cannot rule out underlying suicidal thinking, e.g., a motor vehicle accident, or behaving in a dangerous or unsafe way and other self-injurious behaviors.

General Aspects

Because secondary objectives address maintenance of effect while patients are stable, secondary efficacy analyses will focus on changes from randomization during the time that patients are stable (i.e., prior to relapse). For patients with a documented relapse, all assessments from randomization to the last assessment prior to the event will be used. For patients not experiencing relapse during trial, all available assessments during the randomized treatment phase will be used. Safety objectives will consider both changes from enrolment and changes from randomization.

Method of Statistical Analysis

General aspects: All statistical tests will be two-sided with a significance level or 5%, i.e. α=0.05. Where appropriate, 95% confidence intervals will be presented. Also descriptive statistics will be provided for all variables.

Baseline quetiapine dose at time prior to randomization (i.e., 50, 150, or 300 mg) may be a factor in treatment response. Titrated-dose (as opposed to fixed-dose) trials can lead to paradoxical dose response results. For instance, it is possible that patients who are quetiapine resistant are titrated to the maximum allowed dose, resulting in a subgroup of patients who are a mixture of patients who are responsive to 300 mg quetiapine, and those who are not responsive at all. Such a mixed group of patients would likely show less efficacy for quetiapine after randomization. Similarly, patients titrated to 50 mg quetiapine may be enriched in placebo responders (i.e., patients who would have improved during open label regardless of treatment), resulting in a similar reduction in of treatment response.

In order to avoid confounding treatment effects with baseline quetiapine dose, the randomization will be stratified by baseline quetiapine dose. Since this is a trial of titrated quetiapine versus placebo (and not fixed doses of quetiapine versus placebo and versus each other), the primary analyses will be limited to testing all quetiapine patients versus placebo. Testing for response by baseline dose (and interaction of baseline dose by randomized treatment) will be strictly exploratory.

Time to relapse: The main analysis of the time to relapse of a depressed event will be a stratified Cox proportional hazards model to estimate the hazards ratio of time to relapse of a depressed event between quetiapine and placebo, with 95% confidence intervals. Stratification will be by the randomization strata. This will be a 2-sided test of the null hypothesis, with a statistical significance level of 0.05 using the Wald test statistic.

Time to relapse will be censored at the time the patient discontinues from, or completes the study, without meeting the criteria for relapse. Time of censoring will be the date of the patient's final assessment.

Mean change of Q-les-Q: The outcome variable mean change in Q-les-Q total score will be analyzed using repeated measures, mixed effects analysis with Q-les-Q total score at randomization as a covariate and including treatment as a fixed effect. This analysis will test for an overall mean difference between treatment groups as follows.

After titration from quetiapine therapy to randomized therapy, mean assessments scores between treatment arms are expected to monotonically diverge according to one of the following cases: 1) mean scores gradually diverge over time (i.e., show a pure trend); 2) mean scores quickly diverge after titration then maintain a steady difference (i.e., show a pure jump); and 3) mean scores show a combination of the above (i.e., show both a jump after titration and a gradual trend there after. These cases are alternative to the null hypothesis that there is no difference in trend or jump, i.e., slope or intercept. It is also assumed that treatment arms vary in the length of time participating in the trial (which is the primary trial objective). If it is confirmed that the time-to-relapse differs between treatments, and there is a trend of increasing differences over time, then an analysis that does not compensate for these differences will show some bias (either for or against quetiapine). Consequently, a repeated measures analysis of efficacy measures must take into account trend over time and length of time randomized.

For these reasons, the repeated measures analysis will test for a statistically significant difference between treatment arms using the Least Square Mean (LSM) estimates of the 24-week change from baseline. For patients who complete the trial, this time point would represent the middle of their randomized maintenance treatment period; hence the treatment effect will be an interpolation at the 24 weeks based on observations from both before and after (4, 12, 24, 36 and 48 weeks). It is expected that about 50% of patients will complete the randomized phase (i.e., 25% with depression events, and 25% withdrawn), and perhaps 70% complete 24 weeks. Consequently, the contribution of many patients to the analysis will be weighted toward the earlier visits. (NB: under the proposed model, patients who withdraw prior to 24 weeks will still contribute to the analysis, in that the observed treatment differences will be contribute to estimating the nature of the diverging treatment effect earlier in the randomized treatment period).

The estimated treatment difference at 24 weeks will be an average of the treatment differences before and after that visit, adjusted for differences in time to relapse in the treatment arms. In this analysis all assessments between randomization and up to, but excluding, the visit where a relapse event was recorded will be used. If no relapse was recorded for a patient, all visits after randomization with available Q-les-Q data will be used.

Overall Efficacy

The same statistical modeling of Q-les-Q will be used to analyze the change from randomization MADRS, HAM-A and CGI-S scores. All assessments between randomization and up to, but excluding the relapse, will be included in the analyses.

Each of MADRS, HAM-A, and CGI-S scores is be analyzed using the same methods as in the modeling of the Q-les-Q scores (described above). Other potential covariates will be examined and finalized in the SAP. All assessments between randomization and up to, but excluding the depressed event, will be included in the analyses.

Variables that are dichotomous will be analyzed using a logistic model and the Cochran-Mantel-Haenszel (CMH) test statistic.

Safety analyses: Safety and tolerability will be assessed for the Open-label treatment phase, the randomized treatment phase, and across the entire study interval.

Descriptive statistics of incidence rates will be used to evaluate adverse events (including serious adverse events, adverse events leading to withdrawal, and deaths if any), and reasons for study early withdrawal. Other safety analyses will be by means of descriptive statistics, mean, median, standard deviation, minimum and maximum value, frequency tables and graphs as appropriate.

AIMS, SAS, and BARS: Because patients experiencing movement disorders may be more likely to withdraw from the study, the EPS safety assessments AIMS, SAS, and BARS will be analyzed as change from randomization to last observation carried forward (LOCF). Statistical testing will use mixed effects Analysis of Covariance (ANCOVA) with scores at randomization as a covariate, treatment as fixed effect, and region as a random effect.

### Example 14

### MDD Monotherapy 50 mg/Day, 150 mg/Day and 300 mg/Day

The overall rationale for this study is to evaluate that a sustained release form of quetiapine is efficacious and safe in the treatment of subjects with MDD. This is a 6-week placebo-controlled, randomized study evaluating the efficacy and safety of three fixed doses of a sustained release form of quetiapine given as monotherapy in the treatment of subjects with MDD.

Patient eligibility includes male or female subjects, 18 to 65 years old, with a documented clinical diagnosis using the MINI and meeting the DSM-IV of either: 1) 296.2× MDD, Single Episode; or 2) 296.3× MDD, Recurrent. The patients should also have a HAMD score≧22 to be eligible for the study. In order to obtain a balanced population between moderate and severe MDD the aim for enrolling is a patient population with an average score of 28 on the HAMD.

In some embodiments, the following hypotheses are analyzed: 1) a sustained release form of quetiapine once daily has superior efficacy to placebo in depression; 2) a sustained release form of quetiapine once daily has a greater response rate than placebo in depression; 3) a sustained release form of quetiapine once daily is better than placebo in achieving remission in patients with depression; and/or 4) starting on day 1 A sustained release form of quetiapine once daily can be prescribed at a therapeutically effective dose in depression. A primary objective is to evaluate the efficacy of three doses of a sustained release form of quetiapine versus placebo in patients with MDD. A primary variable is the change from randomization to Week 6 in the Montgomery-Asberg Depression Rating Scale (MADRS) total score. Secondary variables supporting the primary objective include: 1) change from randomization to each assessment in the MADRS total score MADRS response, defined as a >50% reduction from randomization in the MADRS total score at Week 6; 2) MADRS remission, defined as total score≦8 at Week 6; 3) change from randomization to week 6 in the Hamilton Depression scale (HAM-D) total score and the HAM-D Item 1; 4) change from randomization to each assessment in the CGI-S, Clinical Global Impression—Improvement (CGI-J) from randomization to each assessment.

In some embodiments, the following efficacy hypotheses are analyzed: a sustained release form of quetiapine once daily demonstrates an anti-depressive effect by day 4 and a sustained release form of quetiapine once daily has a greater response rate at day 4 than placebo in depression. Secondary objectives include, for example, to evaluate the efficacy of a sustained release form of quetiapine versus placebo at day 4 in patients with MDD and to evaluate if a sustained release form of quetiapine is effective at day 4 in patients with MDD. Outcome variables include, for example: change from randomization to Day 4 in MADRS total score; change from randomization to day 4 in the CGI-S score; and MADRS response, defined as a >50% reduction from randomization in the MADRS total score at Day 4.

In some embodiments, the following efficacy hypotheses are analyzed: a sustained release form of quetiapine once daily is more effective than placebo in reducing anxiety symptoms in depression. Secondary objectives include, for example, to evaluate if a sustained release form of quetiapine reduces anxiety symptoms in patients with MDD, compared to placebo. Outcome variables include, for example: change from randomization to each assessment in HAM-A; change in HAM-A psychic anxiety factors (Anxious Mood, Tension, Fears, Insomnia, Intellect, Depressed Mood, Behaviour at interview) from randomization to each assessment; and change in HAM-D anxiety factors (item 10 and 11) from randomization to Week 6.

In some embodiments, the following efficacy hypotheses are analyzed: a sustained release form of quetiapine once daily is more effective than placebo in improving sleep onset and sleep maintenance in depression. Secondary objectives include, for example, to evaluate if a sustained release form of quetiapine improves sleep quality in patients with MDD, compared to placebo. Outcome variables include, for example: change in HAM-D sleep disturbance factors (Items 4-6) from randomization to Week 6; and change in PSQI global score from randomization to each assessment.

In some embodiments, the following efficacy hypotheses are analyzed: a sustained release form of quetiapine once daily is more effective than placebo in reducing suicide ideation in patients with depression. Secondary objectives include, for example, to evaluate if a sustained release form of quetiapine is effective in reducing suicidal ideation in patients with MDD, compared to placebo. Outcome variables include, for example: change from randomization to each assessment in MADRS item 10, suicidal thought.

In some embodiments, the following efficacy hypotheses are analyzed: a sustained release form of quetiapine once daily is more effective than placebo in improving somatic symptoms such as back pain, headache, muscle pain, unspecified pain, abdominal pain, chest pain, in patients with depression. Secondary objectives include, for example, to evaluate if a sustained release form of quetiapine improves somatic symptoms in the treatment of subjects with MDD, compared to placebo. Outcome variables include, for example: Change in HAM-A somatic anxiety factors (Somatic Muscular, Somatic Sensory, Cardiovascular, Respiratory, Gastrointestinal, Genitourinary, Autonomic) from randomization to each assessment.

In some embodiments, the following efficacy-quality of life hypotheses are analyzed: A sustained release form of quetiapine once daily is more effective than placebo in improving the quality of life of patients with depression. Secondary objectives include, for example, to evaluate if a sustained release form of quetiapine improves the quality of life of patients with MDD, compared to placebo. Outcome variables include, for example: change from baseline to each assessment in Q-les-Q total score (item 1-14); and change from baseline to each assessment in Q-les-Q Item 16 (Overall quality of life).

In some embodiments, the following efficacy-quality of life hypotheses are analyzed: a sustained release form of quetiapine once daily is more effective than placebo in improving patient satisfaction in patients with depression. Secondary objectives include, for example, to evaluate if a sustained release form of quetiapine improves patient satisfaction in patients with MDD, compared to placebo. Outcome variables include, for example: change from randomization to each assessment in Q-les-Q Item 15 (Satisfaction with medication).

In some embodiments, the following safety/tolerability hypotheses are analyzed: a sustained release form of quetiapine once daily up to 300 mg/d is well tolerated in patients with depression; a sustained release form of quetiapine once daily does not have serious discontinuation symptoms; somnolence with a sustained release form of quetiapine once daily is generally mild, occurs early in treatment; is not persistent in the majority of patients and is rarely a cause of withdrawal; fasting glucose and lipids not significantly elevated; a sustained release form of quetiapine once daily is associated with a favourable weight profile; a sustained release form of quetiapine once daily is associated with placebo levels of nausea and vomiting; a sustained release form of quetiapine once daily is associated with placebo levels of EPS (including akathisia); and a sustained release form of quetiapine once daily is associated with a lower incidence of treatment emergent suicidal ideation compared with placebo. Secondary objectives include, for example, to evaluate if a sustained release form of quetiapine is safe and well tolerated in the treatment of subjects with MDD; and to evaluate if a sustained release form of sustained release form of quetiapine is as safe and well-tolerated as placebo in the treatment of subjects with MDD. Outcome variables include, for example: change from normal to Clinically Important in: Physical Examinations, Laboratory values (including glucose/lipids), Vital signs, Electrocardiograms (ECGs); Adverse Events; AEs leading to withdrawal; Serious discontinuation symptoms assessed by DESS (discontinuation scale); AEs related to somnolence; Severity of somnolence reports; Time of AE somnolence reports; Withdrawals due to AE of somnolence; Change in weight from randomization to each assessment; Change in waist circumference from randomization to each assessment; Proportion of patients with a ≧7% increase from randomization weight; AEs (especially related to sexual dysfunction, nausea, vomiting, EPS including akathisia); change in SAS and BARS from randomization to each assessment; MADRS item 10 score>4 at any time after randomization or AE of suicidality/suicidal ideation/suicide attempts/suicide completion; and analysis of suicidality according to FDA guidance.

Subjects are required to have a HAM-D (17-item scale) score of ≧22 at screening and randomization.

The study comprises the following three periods:

1) Washout period: If they qualify to participate, patients will commence a washout of all psychotropic medications. There will be a washout period of at least 7 days in order to discontinue all psychotropic medications before randomization. If subject is not taking psychotropic medications at screening and therefore no washout period is necessary, they may be randomized after confirmation of eligibility. For verification of HAMD scores a system to systematically review the scores will be set up, as to prevent scale inflation.

2) Six-Week randomized, placebo controlled treatment period_(Day 1 to Day 43): Eligible subjects will be randomized on Day 1 (Visit 2) to one of four treatment groups: a sustained release form of quetiapine 50 mg/day, a sustained release form of quetiapine 150 mg/day, a sustained release form of quetiapine 300 mg/day, or placebo. The likelihood of entering the placebo arm is 25%. Subjects will be treated and assessed for 6 weeks.

3) Two-week follow-up period_(Day 44 to Day 57): All randomized patients will be asked to call in through an IVRS system to do an assessment of discontinuation-emergent signs and symptoms (DESS) at 1, 3, 5, 7 and 14 days after their final dose of study medication (Day 44, 46, 48, 50 and 57). No down titration will be needed of a sustained release form of quetiapine.

The subject will be randomized to a double blind treatment with a sustained release form of quetiapine 50 mg/day, a sustained release form of quetiapine 150 mg/day, a sustained release form of quetiapine 300 mg/day or placebo. Tablets to be used in the study are: 50 and 300 mg quetiapine sustained release (SR) tablets and placebo tablets to match.

The sustained release form of quetiapine or placebo will be administered once daily at bedtime. All quetiapine patients will start on 50 mg/day, being uptitrated to 150 mg/day at day 3. The patients in the 300 mg/day treatment group will be increased to 300 mg/day on day 5 (see Table 18).

TABLE 18
Titration of investigational product

| Treatment group | 50 mg/day | 150 mg/day | 300 mg/day | placebo |
|---|---|---|---|---|
| Day 1-2 | 1x 50 mg | 1x 50 mg sustained | 1x 50 mg sustained | 3x 50 mg placebo |
| | sustained release | release form of | release form of | tablets |
| | form of | quetiapine tablets | quetiapine tablets | 1x 300 mg placebo |
| | quetiapine tablets | 2x 50 mg placebo | 2x 50 mg placebo | tablets |
| | 2x 50 mg placebo | tablets | tablets | |
| | tablets | 1x 300 mg placebo | 1x 300 mg placebo | |
| | 1x 300 mg | tablets | tablets | |
| | placebo tablets | | | |
| Day 3-4 | 1x 50 mg | 3x 50 mg sustained | 3x 50 mg sustained | 3x 50 mg placebo |
| | sustained release | release form of | release form of | tablets |
| | form of | quetiapine tablets | quetiapine tablets | 1x 300 mg placebo |
| | quetiapine tablets | 1x 300 mg placebo | 1x 300 mg placebo | tablets |
| | 2x 50 mg placebo | tablets | tablets | |
| | tablets | | | |
| | 1x 300 mg | | | |
| | placebo tablets | | | |
| Day 5-43 | 1x 50 mg | 3x 50 mg sustained | 3x 50 mg placebo | 3x 50 mg placebo |
| | sustained release | release form of | tablets | tablets |
| | form of | quetiapine tablets | 1x 300 mg | 1x 300 mg placebo |
| | quetiapine tablets | 1x 300 mg placebo | sustained release | tablets |
| | 2x 50 mg placebo | tablets | form of quetiapine | |
| | tablets | | tablets | |
| | 1x 300 mg | | | |
| | placebo tablets | | | |

Study Procedures

Eligibility for the study will be assessed at screening and randomization. The subjects will be randomized to treatment groups at Day 1 after fulfilling all inclusion criteria and none of the exclusion criteria. Visit 3 (Day 4) allow a visit window of +I days and for all other visits a visit window of ±2 days calculated from randomization is allowed.

Statistical Analysis

The null hypotheses is that there is no difference between the three quetiapine treatments and the placebo treatment in change in MADRS total score from randomization to Week 6. Each quetiapine dose group (50, 150 mg and 300 mg) will be compared to placebo.

The null hypotheses is that there is no difference between the three quetiapine treatments and the placebo treatment in change in Q-LES-Q total score from randomization to Week 6. Each quetiapine dose group (50, 150 mg and 300 mg) will be compared to placebo.

In order to take account of these 6 comparisons, a parallel gatekeeper approach will be used (see Scheme A). The first family will consist of the two hypotheses connected to quetiapine 150 mg and 300 mg in the primary variable (MADRS). The second family will consist of the hypothesis connected to quetiapine 50 mg in the primary variable (MADRS) together with the two hypotheses connected to quetiapine 150 mg and 300 mg in the secondary variable (Q-LES-Q). The third family will consist of the hypothesis connected to quetiapine 50 mg in the secondary variable (Q-LES-Q).

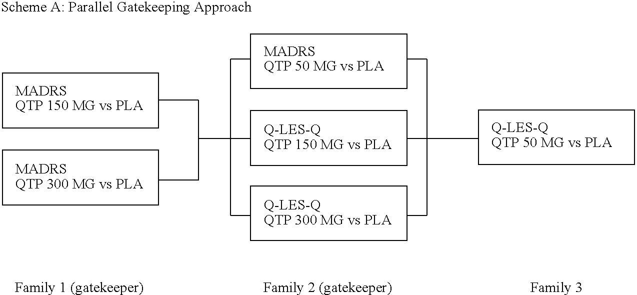

The hypotheses in family 1 will serve as a gatekeeper in the sense that hypotheses in the second family will only be tested if at least one of the tests in family 1 exhibits significance. The hypotheses in the second family will in the same manner serve as gatekeepers for the hypothesis in the third family. Using a gatekeeping strategy for testing the primary and secondary hypotheses will preserve the overall experiment type I error rate at 0.05.

Weights will be applied equally within the first family and set at 0.5. Similarly, weights will be applied equally within second family and set at 0.3333. Since the third family only consists of one hypothesis, no weights will be used.

Analysis Populations

The efficacy analyses will be based on the modified intention-to-treat population (Full Analysis Set). This population will include all randomized subjects, classified according to randomized treatment, who took study medication and who have a baseline MADRS assessment and at least 1 valid MADRS assessment after baseline.

The safety displays will be based on the safety population. This population include all randomized subjects who took study medication, classified according to the treatment actually received.

Analysis of the Primary Outcome Variable

The primary outcome variable, the change in MADRS total score from randomization to Week 6, will be analyzed using a mixed model analysis with MADRS total score at randomization as a covariate and including treatment as a fixed effect and centre as a random effect. The comparisons of interest will be the difference between each sustained release form of quetiapine dose and placebo. A parallel gatekeeping approach will be used to adjust for multiple comparisons as described above.

Secondary Efficacy Analysis of Primary Interest

The outcome variable, the change in Q-LES-Q total score from randomization to Week 6, will be analyzed using a mixed model analysis with Q-LES-Q total score at randomization as a covariate and including treatment as a fixed effect and centre as a random effect. The comparisons of interest will be the difference between each sustained release form of quetiapine dose and placebo. A parallel gatekeeping approach will be used to adjust for multiple comparisons as described above.

The sample size calculation in this study was done to demonstrate superior efficacy of the 150 mg and/or the 300 mg sustained release form of quetiapine doses over placebo and where calculated with regard to the primary outcome variable, change in MADRS total score from baseline to Week 6. A 2-sided test at a=0.025 for the two comparisons of sustained release form of quetiapine versus placebo using an anticipated difference of 3.5 unit difference from placebo and a within patient variability (standard deviation) of 9 for the change in MADRS total score from baseline to Week 6 ensures an individual power of 90% for the two high doses. This yields a planned sample size of 166 for each of the four arms, and 664 in total.

Assuming that 93% of all randomized patients are expected to be evaluable patients (to be included in MITT), a total of about 712 randomized patients are required to obtain 166 evaluable patients per treatment group.

Exemplary sample size calculations are shown in Table 19.

TABLE 19

| | As specified |
|---|---|
| Individual power | 90% |
| Difference to be detected | 3.5 |
| compared to placebo | |
| Standard deviation | 9 |
| Overall Significance level | 0.05 |
| Sample size (evaluable) | 166 |

MDD Exclusion criteria, includes but is not limited to, the criteria shown in Table 20.

TABLE 20

| MDD Exclusion Criteria | | Rationale |
|---|---|---|
| 1. | Subjects with a DSM-IV Axis I disorder other | To exclude other diagnoses which may |
| | than MDD within 6 months of enrolment, | confound results. |
| 2. | Subjects whose current episode of depression | To exclude treatment-resistant subjects and |
| | exceeds 12 months or is less than 4 weeks from | subjects with incorrect diagnoses. |
| | enrolment. | |
| 3. | History of in-adequate response to an adequate | To exclude treatment-resistant subjects. |
| | treatment (6 weeks) with 2 or more classes of | |
| | antidepressants during current depressive episode. | |
| 4. | Substance or alcohol abuse or dependence within | To exclude subjects with active substance |
| | 6 months prior to screening (except dependence in | abuse, which may interfere with assessments |
| | full remission, and except for caffeine or nicotine | of mood. |
| | dependence), as defined in DSM-IV criteria. | |
| | Subjects with a positive urine toxicology screen | |
| | will be excluded. Patients can be re-tested if | |
| | positive initial UTS, but should be excluded if still | |
| | positive at second test. | |
| 5. | Use of drugs that induce or inhibit the hepatic | To ensure more consistent levels of study drug |
| | metabolizing cytochrome 3A4 enzymes within 2 | across subject populations. |
| | weeks prior to randomization (e.g. inducers: | |
| | carbamazepine, phenytoin, barbiturates, rifampin, | |
| | rifabutin, glucocorticoids, thioridazine and St | |
| | John's wort, and inhibitors: ketoconazole (except | |
| | for topical use), itraconazole, fluconazole, | |
| | erythromycin, clarithromycin, fluvoxamine, | |
| | nefazodone, troleandomycin, indinavir, nelfinavir, | |
| | ritonavir, and saquinavir). | |
| 6. | Evidence of clinically relevant disease, (e.g. renal | Potentially confounds results. To ensure |
| | or hepatic impairment, significant coronary artery | safety. |
| | disease, cerebrovascular disease, viral hepatitis B | |
| | or C, acquired immunodeficiency syndrome | |
| | [AIDS), or a clinical finding that is unstable or | |
| | that, in the opinion of the investigator, would be | |
| | negatively affected by the study medication or that | |
| | would affect the study medication | |
| 7. | Use of antipsychotic, mood stabilizer, or | To ensure that previous psychotropic drugs do |
| | antidepressant drugs within 7 days before | not affect study assessments. |
| | randomization, or use of fluoxetine within 28 days | |
| | before randomization, or use of MAO inhibitors, | |
| | anxiolytic or hypnotics within 14 days before | |
| | randomization (with the exception of those | |
| | allowed with restriction per protocol), or use of a | |
| | depot antipsychotic injection within two dosing | |
| | interval before randomization. | |
| 8. | Subjects who in the investigators opinion will | To prevent new therapies being introduced |
| | require psychotherapy (other then supportive | during study treatment period |
| | psychotherapy) during the study period, unless | |
| | psychotherapy has been ongoing for a minimum | |
| | of 3 months prior to randomization | |
| 9. | Subjects who, in the investigator's judgment pose | To ensure that subjects at high risk of suicide |
| | a current serious suicidal or homicidal risk, have a | are not being treated in the study |
| | HAM-D item 3 score of 3 or greater, or have made | |
| | a suicide attempt within the past 6 months. | |
| 10. | Known lack of response to quetiapine in the | To avoid known non-responders in the study. |
| | treatment of depression in a dosage of at least 150 mg/ | |
| | day for 4 weeks, as judged by the investigator. | |

Exemplary restrictions in treatments are shown in Table 21.

TABLE 21

| Restrictions | Rationale |
|---|---|
| Prohibited | |
| Use of drugs that induce or inhibit the hepatic | Potentially confounds the results. |
| metabolizing cytochrome 3A4 enzymes within 2 | To ensure safety. |
| weeks prior to randomization (e.g. inducers: | |
| carbamazepine, phenytoin, barbiturates, rifampin, | |
| rifabutin, glucocorticoids, thioridazine and St | |
| John's wort, and inhibitors: ketoconazole (except | |
| for topical use), itraconazole, fluconazole, | |
| erytromycin, clarithomycin, fluvoxamine, | |
| nefazodone, troleandomycin, indinavir, nelfinavir, | |
| ritonavir, and saquinavir). | |
| Use of any psychoactive drugs including hypnotic, | Potentially confounds the results. |
| antidepressant, anxiolytic, mood stabilizing, | |
| antipsychotic, and sedative medications other than | |
| restricted. | |
| Electroconvulsive therapy (ECT) throughout the | Potentially confounds the results. |
| randomized treatment period. | |
| Abuse according to the DSM-IV criteria of Alcohol, | Potentially confounds the results. |
| Opiates, amphetamine, barbiturate, cocaine, | |
| cannabis, or hallucinogen throughout the study | |
| Restricted | |
| One of the following can be used for insomnia (at | Need to allow hypnotics at reasonable doses. |
| bedtime) up to the specified dosage per night if | Differences in treatment traditions and availability of |
| treatment has been ongoing since 30 days prior | products between countries require alternative |
| enrolment on a regular basis as judged by the | products. |
| investigator: lorazepam max 2 mg/day; zolpidem | |
| tartrate 10 mg; zaleplon, 20 mg; zopiclone, 7.5 mg; | |
| chloral hydrate, 1 g. Hypnotic use not allowed on | |
| the night prior to conducting study assessments. | |
| Psychotherapy is only allowed if it has been | |
| ongoing since at least 3 months prior to | |
| randomization. | |
| Permitted | |
| Nonpsychoactive medications, including over-the- | patients may require medications to treat underlying |
| counter medications which are required to treat | medical conditions |
| illness or complaints that occur during the study | |

### Example 15

### 150 and 300 mg/day as Monotherapy in Treatment of MDD

This study is a 6 week randomized treatment with two weeks follow-up after end of treatment period. The overall rationale for this study is to evaluate that quetiapine sustained release is efficacious and safe in the treatment of patients with MDD. This trial will investigate the short-term efficacy and safety of sustained release form of quetiapine in MDD, and provide information regarding the most appropriate dose.

The primary objective of the study is to evaluate superior efficacy of sustained release form of quetiapine compared with placebo and duloxetine in the treatment of patients with MDD (Table 19). The secondary objectives are shown in Table 23.

Patient eligibility includes male or female subjects, 18 to 65 years old, with a documented clinical diagnosis using the MINI and meeting the DSM-IV of either: 1) 296.2× MDD, Single Episode; or 2) 296.3× MDD, Recurrent. The patients should also have a HAMD score≧22 to be eligible for the study. In order to obtain a balanced population between moderate and severe MDD the aim for enrolling is a patient population with an average score of 28 on the HAMD.

TABLE 22
Primary objective, corresponding outcome variables and claims

| Claims to be addressed | Primary Objective | Outcome Variable |
|---|---|---|
| 2.2 A sustained release form | To evaluate the efficacy of | Primary variable: |
| of quetiapine once daily has | sustained release form of | Change from randomization to Week 6 in |
| superior efficacy to placebo in | quetiapine versus placebo in | the Montgomery-Åsberg Depression |
| depression | patients with MDD. | Rating Scale (MADRS) total score |
| 2.4 A sustained release form | | Secondary variables supporting the |
| of quetiapine once daily has a | | primary objective: |
| greater response rate than | | Change from randomization to each |
| placebo in depression | | assessment in the MADRS total score |
| 2.6 A sustained release form | | MADRS response, defined as a ≧50% |
| of quetiapine once daily is | | reduction from randomization in the |
| better than placebo in | | MADRS total score at Week 6. |
| achieving remission in patients | | MADRS remission, defined as total score |
| with depression | | ≦8 at Week 6. |
| 9.1 Starting on day 1 A | | Change from from randomization to week |
| sustained release form of | | 6 in the Hamilton Depression Scale |
| quetiapine once daily can be | | (HAM-D) total score and the HAM-D |
| prescribed at a therapeutically | | Item 1. |
| effective dose in depression | | Change from |
| | | randomization to each assessment in the |
| | | Clinical Global Impression —Severity |
| | | (CGI-S) |
| | | Clinical Global Impression —Improvement |
| | | (CGI-I) from randomization to each |
| | | assessment |

TABLE 23
Secondary objectives, corresponding outcome variables and claims

| Claims to be addressed or | | |
|---|---|---|
| hypothesis to be tested | Secondary Objective | Outcome Variables |
| Efficacy | | |
| 2.1 A sustained release form | To evaluate the efficacy of | Change from randomization to each |
| of quetiapine once daily is at | sustained release form of | assessment in the MADRS total score |
| least as effective as | quetiapine compared to | MADRS response, defined as a ≧50% |
| duloxetine in depression | duloxetine in the treatment of | reduction from randomization in the MADRS |
| 2.3 A sustained release form | patients with MDD. | total score at Week 6. |
| of quetiapine once daily has | | MADRS remission, defined as total score ≦8 |
| a response rate at least as | | at Week 6. |
| great as duloxetine in | | Change from randomization to week 6 in the |
| depression | | HAM-D total score and the HAM-D Item 1 |
| 2.5 A sustained release form | | Change from randomization to each |
| of quetiapine once daily is at | | assessment in the CGI-S |
| least as effective as | | Improvement in CGI-I from randomization |
| duloxetine in achieving | | to each assessment |
| remission in patients with | | |
| depression | | |
| 3.1 A sustained release form | To evaluate if sustained | Change from randomization to each |
| of quetiapine once daily is at | release form of quetiapine | assessment in Hamilton Rating scale for |
| least as effective as | reduces anxiety symptoms in | Anxiety (HAM-A) |
| duloxetine in reducing | patients with MDD, compared | Change in HAM-A psychic anxiety factors |
| anxiety symptoms in | to duloxetine. | (Anxious Mood, Tension, Fears, Insomnia, |
| depression | | Intellect, Depressed Mood, Behaviour at |
| 3.2 A sustained release form | To evaluate if sustained | interview) from randomization to each |
| of quetiapine once daily is | release form of quetiapine | assessment |
| more effective than placebo | reduces anxiety symptoms in | Change in HAM-D anxiety factors (item 10 |
| in reducing anxiety | patients with MDD, compared | and 11) from randomization to Week 6 |
| symptoms in depression | to placebo | |
| 4.1 A sustained release form | To evaluate if sustained | Change in HAM-D sleep disturbance factors |
| of quetiapine once daily is at | release form of quetiapine | (Items 4-6) from randomization to Week 6 |
| least as effective as | improves sleep quality in | Change in Pittsburgh Sleep Quality Index |
| duloxetine in improving | patients with MDD, compared | (PSQI) global score from randomization to |
| sleep onset and sleep | to duloxetine. | each assessment |
| maintenance in depression | | |
| 4.2 A sustained release form | To evaluate if sustained | |
| of quetiapine once daily is | release form of quetiapine | |
| more effective than placebo | improves sleep quality in | |
| in improving sleep onset and | patients with MDD, compared | |
| sleep maintenance in | to placebo. | |
| depression | | |
| 5.1 A sustained release form | To evaluate if sustained | Change from randomization to each |
| of quetiapine once daily is at | release form of quetiapine is | assessment in MADRS item 10, suicidal |
| least as effective as | effecitve in reducing suicidal | thought |
| duloxetine in reducing | ideation in patients with | |
| suicide ideation in patients | MDD, compared to | |
| with depression | duloxetine. | |
| 5.2 A sustained release form | To evaluate if sustained | |
| of quetiapine once daily is | release form of quetiapine is | |
| more effective than placebo | effective in reducing suicidal | |
| in reducing suicide ideation | ideation in patients with | |
| in patients with depression | MDD, compared to placebo. | |
| 8.1 A sustained release form | To evaluate if sustained | Change in HAM-A somatic anxiety factors |
| of quetiapine once daily is at | release form of quetiapine | (Somatic Muscular, Somatic Sensory, |
| least as effective as | improves somatic symptoms | Cardiovascular, Respiratory, Gastrointestinal, |
| duloxetine in improving | in the treatment of patients | Genitourinary, Autonomic) from |
| somatic symptoms such as | with MDD, compared to | randomization to each assessment. |
| back pain, headache, muscle | duloxetine. | |
| pain, unspecified pain, | | |
| abdominal pain, chest pain, | | |
| in patients with depression | | |
| 8.2 A sustained release form | To evaluate if sustained | |
| of quetiapine once daily is | release form of quetiapine | |
| more effective than placebo | improves somatic symptoms | |
| in improving somatic | in the treatment of subjects | |
| symptoms such as back pain, | with MDD, compared to | |
| headache, muscle pain, | placebo.. | |
| unspecified pain, abdominal | | |
| pain, chest pain, in patients | | |
| with depression | | |
| Efficacy - Quality of Life | | |
| 6.1 A sustained release form | To evaluate if sustained | Change from baseline to each assessment in |
| of quetiapine once daily is at | release form of quetiapine | Q-les-Q total score (item 1-14). |
| least as effective as | improves the quality of life of | Change from baseline to each assessment in |
| duloxetine in improving the | patients with MDD, compared | Q-les-Q Item 16 (Overall quality of life). |
| quality of life of patients | to duloxetine. | |
| with depression | | |
| 6.2 A sustained release form | To evaluate if sustained | |
| of quetiapine once daily is | release form of quetiapine | |
| more effective than placebo | improves the quality of life of | |
| in improving the quality of | patients with MDD, compared | |
| life of patients with | to placebo. | |
| depression | | |
| 7.1 A sustained release form | To evaluate if sustained | Change from randomization to each |
| of quetiapine once daily is at | release form of quetiapine | assessment in Q-les-Q Item 15 (Satisfaction |
| least as effective as | improves patient satisfaction | with medication) |
| duloxetine in improving | in patients with MDD, | |
| patient satisfaction in | compared to duloxetine. | |
| patients with depression | | |
| 7.2 A sustained release form | To evaluate if sustained | |
| of quetiapine once daily is | release form of quetiapine | |
| more effective than placebo | improves patient satisfaction | |
| in improving patient | in patients with MDD, | |
| satisfaction in patients with | compared to placebo. | |
| depression | | |
| Safety/tolerability | | |
| 11.1 A sustained release | To evaluate if sustained | Change from normal to Clinically Important |
| form of quetiapine once daily | release form of quetiapine is | in: |
| up to 300 mg/d is well | safe and well tolerated in the | Physical Examinations |
| tolerated in patients with | treatment of patients with | Laboratory values (including |
| depression | MDD. | glucose/lipids) |
| 11.8 A sustained release | | Vital signs |
| form of quetiapine once daily | | Electrocardiograms (ECGs) |
| does not have serious | | Adverse Events |
| discontinuation symptoms | | AEs leading to withdrawal |
| 11.9 Somnolence with | | Serious discontinuation symptoms assessed |
| sustained release form of | | by DESS (discontinuation scale) |
| quetiapine once daily is | | AEs related to somnolence |
| generally mild, occurs early | | Severity of somnolence reports |
| in treatment; is not persistent | | Time of AE somnolence reports |
| in the majority of patients | | Withdrawals due to AE of somnolence |
| and is rarely a cause of | | Change in weight from randomization to each |
| withdrawal | | assessment |
| 11.10 Fasting Glucose and | | Change in waist circumference from |
| Lipids not significantly | | randomization to each assessment |
| elevated | | Proportion of patients with a ≧7% increase |
| 11.13 A sustained release | | from randomization weight. |
| form of quetiapine once daily | | |
| is associated with a favorable | | |
| weight profile | | |
| 11.14 A sustained release | | |
| form of quetiapine once daily | | |
| has a comparable incidence | | |
| of withdrawals due to | | |
| adverse events than | | |
| duloxetine | | |
| 11.2 A sustained release | For 11.2, 11.4, 11.6, and 11.12 | AEs (especially related to sexual dysfunction, |
| form of quetiapine once daily | To evaluate if sustained | nausea, vomiting, EPS including akathisia) |
| is associated with placebo | release form of quetiapine is | Change from randomization to each |
| levels of sexual dysfunction | as safe and well-tolerated as | assessment in Sexual Functioning |
| | placebo in the treatment of | Questionnaire (CSFQ) total score |
| | patients with MDD | Change in SAS and BARS from |
| 11.3 A sustained release | For 11.3, 11.5, 11.11 | randomization to each assessment |
| form of quetiapine once daily | To evaluate if sustained | MADRS item 10 score ≧4 at any time after |
| is associated with lower | release form of quetiapine is | randomization or AE of suicidality/suicidal |
| levels of sexual dysfunction | safer and more well-tolerated | ideation/suicide attempts/suicide completion |
| than with duloxetine | than duloxetine in the | Analysis of suicidality according to FDA |
| | treatment of patients with | guidance. |
| | MDD | |
| 11.4 A sustained release | | |
| form of quetiapine once daily | | |
| is associated with placebo | | |
| levels of nausea and | | |
| vomiting | | |
| 11.5 A sustained release | | |
| form of quetiapine once daily | | |
| is associated with lower | | |
| levels of nausea and | | |
| vomiting than duloxetine | | |
| 11.6 A sustained release | | |
| form of quetiapine once daily | | |
| is associated with placebo | | |
| levels of EPS (including | | |
| akathisia) | | |
| 11.11 A sustained release | | |
| form of quetiapine once daily | | |
| is associated with a lower | | |
| incidence of treatment | | |
| emergent suicidal ideation | | |
| compared with duloxetine | | |
| 11.12 A sustained release | | |
| form of quetiapine once daily | | |
| is associated with a lower | | |
| incidence of treatment | | |
| emergent suicidal ideation | | |
| compared with placebo | | |

The study comprises the following three periods. See 1) “Washout Period”, 2) “Six-Week double-blind, randomized, placebo controlled treatment period” from Example 14 above.
Two-Week Follow-Up Period Including One Week Down-Titration (Day 44 to Day 57)

All randomized patients will be asked to call in through an IVRS system to do an assessment of discontinuation-emergent signs and symptoms (DESS) at 1, 3, 5, 7 and 14 days after their final dose of study medication (Day 44, 46, 48, 50 and 57). Patients in the 300 mg/day sustained release form of quetiapine group and patients in the 60 mg/day duloxetine group will be down-titrated during the first follow-up week (see Table 21).

About 600 patients will be randomized to obtain 140 evaluable patients per treatment group (sustained release form of quetiapine 300 mg, sustained release form of quetiapine 150 mg, duloxetine 60 mg and placebo arms) in a 1:1:1:1 randomization. An evaluable patient is a patient with at least one valid post-randomization MADRS assessment completed.

The patient will be randomized to a double blind treatment with sustained release form of quetiapine 150 mg/day, sustained release form of quetiapine 300 mg/day, duloxetine 60 mg/day or placebo.

Tablets and capsules to be used in the study are: 50 and 300 mg quetiapine sustained release (SR) tablets; placebo tablets to match; encapsulated duloxetine 30 mg capsules; placebo capsules to match.

The sustained release form of quetiapine, duloxetine or placebo will be administered once daily at bedtime. All sustained release form of quetiapine patients will start on 50 mg/day, being uptitrated to 150 mg/day at day 3. The patients in the 300 mg/day treatment group will be increased to 300 mg/day on day 5. Duloxetine patients can start on 60 mg/day.

TABLE 24
Titration of investigational product

| | sustained release | sustained release | | |
|---|---|---|---|---|
| | form of | form of | | |
| Treatment group | quetiapine | quetiapine | Duloxetine | |
| Day | 150 mg/day | 300 mg/day | 60 mg/day | placebo |
| Day 1-2 | 1x 50 mg | 1x 50 mg | 3x 50 mg placebo | 3x 50 mg placebo |
| | sustained release | sustained release | tablets | tablets |
| | form of quetiapine | form of quetiapine | 1x 300 mg | 1x 300 mg |
| | tablets | tablets | placebo tablets | placebo tablets |
| | 2x 50 mg placebo | 2x 50 mg placebo | 2x 30 mg | 2x 30 mg placebo |
| | tablets | tablets | duloxetine | capsules |
| | 1x 300 mg | 1x 300 mg | capsules | |
| | placebo tablets | placebo tablets | | |
| | 2x 30 mg placebo | 2x 30 mg placebo | | |
| | capsules | capsules | | |
| Day 3-4 | 3x 50 mg | 3x 50 mg | 3x 50 mg placebo | 3x 50 mg placebo |
| | sustained release | sustained release | tablets | tablets |
| | form of quetiapine | form of quetiapine | 1x 300 mg | 1x 300 mg |
| | tablets | tablets | placebo tablets | placebo tablets |
| | 1x 300 mg | 1x 300 mg | 2x 30 mg | 2x 30 mg placebo |
| | placebo tablets | placebo tablets | duloxetine | capsules |
| | 2x 30 mg placebo | 2x 30 mg placebo | capsules | |
| | capsules | capsules | | |
| Day 5-43 | 3x 50 mg | 3x 50 mg placebo | 3x 50 mg placebo | 3x 50 mg placebo |
| | sustained release | tablets | tablets | tablets |
| | form of quetiapine | 1x 300 mg | 1x 300 mg | 1x 300 mg |
| | tablets | sustained release | placebo tablets | placebo tablets |
| | 1x 300 mg | form of quetiapine | 2x 30 mg | 2x 30 mg placebo |
| | placebo tablets | tablets | duloxetine | capsules |
| | 2x 30 mg placebo | 2x 30 mg placebo | capsules | |
| | capsules | capsules | | |

After 6 weeks of treatment the patients should be down titrated according to the following schedule (see Table 25).

TABLE 25
Down titration of investigational product

| | sustained release | sustained release | | |
|---|---|---|---|---|
| | form of | form of | | |
| Treatment group | quetiapine | quetiapine | duloxetine | |
| Day | 150 mg/day | 300 mg/day | 60 mg/day | placebo |
| Day 44-50 | 3x 50 mg placebo | 3x 50 mg | 3x 50 mg placebo | 3x 50 mg placebo |
| | tablets | sustained release | tablets | tablets |
| | 1x 30 mg placebo | form of quetiapine | 1x 30 mg | 1x 30 mg placebo |
| | capsules | tablets | duloxetine | capsules |
| | | 1x 30 mg placebo | capsules | |
| | | capsules | | |
| Day 51-57 | No treatment | No treatment | No treatment | No treatment |

Statistical Analysis:

Primary Objective:

The null hypothesis is that there is no difference between the two quetiapine treatments and the placebo treatment in change in MADRS total score from randomization to Week 6. Each quetiapine dose group (150 mg and 300 mg) will be compared to placebo.

Secondary Objective of Particular Interest:

The null hypothesis is that there is no difference between the two quetiapine treatments and the placebo treatment in change in Q-LES-Q total score from randomization to Week 6. Each quetiapine dose group (150 mg and 300 mg) will be compared to placebo.

A step-wise sequential testing procedure will be used to handle multiple comparisons across the two groups of hypotheses to ensure that the overall significance level of 0.05 is preserved. First, the primary outcome variable change in MADRS total score from randomization to Week 6 will be tested for each dose versus placebo respectively. If both the quetiapine doses are statistically significantly better than the placebo group, then the hypotheses related to the variable change in Q-LES-Q total score from baseline to Week 6 will be tested for each dose respectively. To handle multiplicity within each step, the Simes-Hommel procedure will be used. 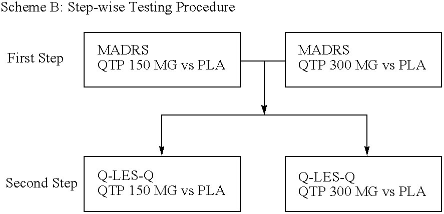
Analysis Populations

The efficacy analyses will be based on the modified intention-to-treat population (Full Analysis Set). This population will include all randomized subjects, classified according to randomized treatment, who took study medication and who have a randomization MADRS assessment and at least 1 valid MADRS assessment after randomization.

The safety displays will be based on the safety population. This population includes all randomized subjects who took study medication, classified according to the treatment actually received.

Primary Efficacy Analysis

The primary outcome variable, the change in MADRS total score from randomization to Week 6, will be analyzed using a mixed model analysis with MADRS total score at randomization as a covariate and including treatment as a fixed effect and centre as a random effect. The comparisons of interest will be the difference between each sustained release form of quetiapine dose and placebo.

The estimated effect and corresponding 95% confidence interval for the change from baseline in MADRS score at Week 6 between duloxetine and placebo will also be provided.

Secondary Efficacy Analysis of Primary Interest

The outcome variable, the change in Q-LES-Q total score from randomization to Week 6, will be analyzed using a mixed model analysis with Q-LES-Q total score at randomization as a covariate and including treatment as a fixed effect and centre as a random effect. The comparisons of interest will be the difference between each sustained release form of quetiapine dose and placebo.

The estimated effect and corresponding 95% confidence interval for the change from baseline in Q-LES-Q total score at Week 6 between duloxetine and placebo will also be provided.

The sample size calculation in this study was done to ensure an 80% power in demonstrating superior efficacy of each of the two sustained release form of quetiapine doses over placebo with regard to the primary outcome variable, change in MADRS total score from baseline to Week 6. Then, the appropriate sample size was attained by assuming an anticipated difference of 3.5 units from placebo and a within patient variability (standard deviation) of 9 for the change in MADRAS total score from baseline to Week 6. Using a two-sided test at a 5% significance level, this yields a planned sample size of 140/arm, and 560 in total to ensure a power of 90% in each individual comparison and an overall power of at least 80%.

Assuming that 93% of all randomized patients are expected to be evaluable patients (to be included in MITT), a total of about 600 randomized patients are required to obtain 140 evaluable patients per treatment group.

An exemplary sample size calculation is shown in Table 26.

TABLE 26
Sample size calculation

| | As specified |
|---|---|
| Over all Power/individual | 80%/90% |
| power | |
| Difference to be detected | 3.5 |
| compared to placebo | |
| Standard deviation | 9 |
| Overall Significance level | 0.05 |
| Sample size (evaluable) | 140 |

Note that the study is not powered for a comparison between sustained release form of quetiapine and duloxetine, only descriptive statistics comparisons will be provided for this comparison.
MDD Exclusion criteria (See “Exclusion criteria” in Example 14 above)
Restrictions in treatments (see “Restrictions” in Example 14 above)

### Example 16

### MDD—Monotherapy 150 mg and 300 mg

The overall rationale for this study is to evaluate that quetiapine fumarate sustained release is efficacious and safe in the treatment of patients with MDD. This trial will serve as one of several studies to investigate the short-term efficacy and safety of quetiapine in MDD.

The primary objective of the study is to evaluate superior efficacy of sustained release form of quetiapine compared with placebo in the treatment of patients with MDD (Table 24). The secondary objectives of the study are shown in Table 25. Escitalopram is added to the study as an active control.

This is an 8-week multi-centre, parallel group, placebo-controlled, randomized study evaluating the efficacy and safety of sustained release form of quetiapine given as monotherapy in the treatment of patients with MDD. Patients are required to have a HAM-D (17-item scale) score of ≧22 at screening and randomization.

See “Patient Eligibility” for Example 15.

TABLE 27
Primary objective, corresponding outcome variables and claims

| Claims to be addressed | Primary Objective | Outcome Variable |
|---|---|---|
| 2.2 A sustained release form of | To evaluate the efficacy of | Primary variable: |
| quetiapine once daily has | sustained release form of | Change from randomization to Week 8 in |
| superior efficacy to placebo in | quetiapine versus placebo in | the MADRS total score |
| depression | patients with MDD. | Secondary variables supporting the |
| 2.4 A sustained release form of | | primary objective: |
| quetiapine once daily has a | | Change from randomization to each |
| greater response rate than | | assessment in the MADRS total score. |
| placebo in depression | | MADRS response, defined as a ≧50% |
| 2.6 A sustained release form of | | reduction from randomization in the |
| quetiapine once daily is better | | MADRS total score at Week 8. |
| than placebo in achieving | | MADRS remission, defined as total score |
| remission in patients with | | ≦8 at Week 8. |
| depression | | Change from randomization to week 8 in |
| 9.1 Starting on day 1 A | | the HAM-D total score and the HAM-D |
| sustained release form of | | Item 1. |
| quetiapine once daily can be | | Change from randomization to each |
| prescribed at a therapeutically | | assessment in the CGI-S |
| effective dose in depression | | CGI-I at Week 8 |

TABLE 28
Secondary objectives, corresponding outcome variables and claims

| Claims to be addressed or | | |
|---|---|---|
| hypothesis to be tested | Secondary Objective | Outcome Variables |
| Efficacy | | |
| 2.1 A sustained release form of | To evaluate the efficacy of | Change from randomization to each |
| quetiapine once daily is at least | sustained release form of | assessment in the MADRS total score |
| as effective as escitalopram in | quetiapine compared to | MADRS response, defined as a ≧50% |
| depression | escitalopram in the treatment of | reduction from randomization in the |
| 2.3 A sustained release form of | patients with MDD. | MADRS total score at Week 8. |
| quetiapine once daily has a | | MADRS remission, defined as total score |
| response rate at least as great as | | ≦8 at Week 8. |
| escitalopram in depression | | Change from randomization to week 8 in |
| 2.5 A sustained release form of | | the HAM-D total score and the HAM-D |
| quetiapine once daily is at least | | Item 1 |
| as effective as escitalopram in | | Change from randomization to each |
| achieving remission in patients | | assessment in the CGI-S |
| with depression | | Improvement in CGI-I from |
| | | randomization to each assessment |
| 3.1 A sustained release form of | To evaluate if sustained release | Change from randomization to each |
| quetiapine once daily is at least | form of quetiapine reduces | assessment in Hamilton Rating scale for |
| as effective as escitalopram in | anxiety symptoms in patients | Anxiety (HAM-A) |
| reducing anxiety symptoms in | with MDD, compared to | Change in HAM-A psychic anxiety |
| depression | escitalopram. | factors (Anxious Mood, Tension, Fears, |
| 3.2 A sustained release form of | To evaluate if sustained release | Insomnia, Intellect, Depressed Mood, |
| quetiapine once daily is more | form of quetiapine reduces | Behaviour at interview) from |
| effective than placebo in | anxiety symptoms in patients | randomization to each assessment |
| reducing anxiety symptoms in | with MDD, compared to | Change in HAM-D anxiety factors (item |
| depression | placebo. | 10 and 11) from randomization to Week |
| | | 8. |
| 4.1 A sustained release form of | To evaluate if sustained release | Change in HAM-D sleep disturbance |
| quetiapine once daily is at least | form of quetiapine improves | factors (Items 4-6) from randomization to |
| as effective as escitalopram in | sleep quality in patients with | Week 8 |
| improving sleep onset and | MDD, compared to | Change in Pittsburgh Sleep Quality Index |
| sleep maintenance in | escitalopram. | (PSQI) global score from randomization |
| depression | | to each assessment |
| 4.2 A sustained release form of | To evaluate if sustained release | |
| quetiapine once daily is more | form of quetiapine improves | |
| effective than placebo in | sleep quality in patients with | |
| improving sleep onset and | MDD, compared to placebo. | |
| sleep maintenance in | | |
| depression | | |
| 5.1 A sustained release form of | To evaluate if sustained release | Change from randomization to each |
| quetiapine once daily is at least | form of quetiapine is effective in | assessment in MADRS item 10, suicidal |
| as effective as escitalopram in | reducing suicidal ideation in | thought |
| reducing suicide ideation in | patients with MDD, compared to | |
| patients with depression | escitalopram. | |
| 5.2 A sustained release form of | To evaluate if sustained release | |
| quetiapine once daily is more | form of quetiapine is effective in | |
| effective than placebo in | reducing suicidal ideation in | |
| reducing suicide ideation in | patients with MDD, compared to | |
| patients with depression | placebo. | |
| 8.1 A sustained release form of | To evaluate if sustained release | Change in HAM-A somatic anxiety |
| quetiapine once daily is at least | form of quetiapine improves | factors (Somatic Muscular, Somatic |
| as effective as escitalopram in | somatic symptoms in the | Sensory, Cardiovascular, Respiratory, |
| improving somatic symptoms | treatment of patients with MDD, | Gastrointestinal, Genitourinary, |
| such as back pain, headache, | compared to escitalopram. | Autonomic) from randomization to each |
| muscle pain, unspecified pain, | | assessment. |
| abdominal pain, chest pain, in | | |
| patients with depression | | |
| 8.2 A sustained release form of | To evaluate if sustained release | |
| quetiapine once daily is more | form of quetiapine improves | |
| effective than placebo in | somatic symptoms in the | |
| improving somatic symptoms | treatment of patients with MDD, | |
| such as back pain, headache, | compared to placebo. | |
| muscle pain, unspecified pain, | | |
| abdominal pain, chest pain, in | | |
| patients with depression | | |
| Efficacy-Quality of Life | | |
| 6.1 A sustained release form of | To evaluate if sustained release | Change from baseline to each assessment |
| quetiapine once daily is at least | form of quetiapine improves the | in Q-les-Q total score (item 1-14). |
| as effective as escitalopram in | quality of life of patients with | Change from baseline to each assessment |
| improving the quality of life of | MDD, compared to | in Q-les-Q Item 16 (Overall quality of |
| patients with depression | escitalopram. | life). |
| 6.2 A sustained release form of | To evaluate if sustained release | |
| quetiapine once daily is more | form of quetiapine improves the | |
| effective than placebo in | quality of life of patients with | |
| improving the quality of life of | MDD, compared to placebo. | |
| patients with depression | | |
| 7.1 A sustained release form of | To evaluate if sustained release | Change from randomization to each |
| quetiapine once daily is at least | form of quetiapine improves | assessment in Q-les-Q Item 15 |
| as effective as escitalopram in | patient satisfaction in patients | (Satisfaction with medication) |
| improving patient satisfaction | with MDD, compared to | |
| in patients with depression | escitalopram. | |
| 7.2 A sustained release form of | To evaluate if sustained release | |
| quetiapine once daily is more | form of quetiapine improves | |
| effective than placebo in | patient satisfaction in patients | |
| improving patient satisfaction | with MDD, compared to | |
| in patients with depression | placebo. | |
| Safety/tolerability | | |
| 11.1 A sustained release form | To evaluate if sustained release | Change from normal to Clinically |
| of quetiapine once daily up to | form of quetiapine is safe and | Important in: |
| 300 mg/d is well tolerated in | well tolerated in the treatment of | Physical Examinations |
| patients with depression | patients with MDD. | Laboratory values (including |
| 11.8 A sustained release form | | glucose/lipids) |
| of quetiapine once daily does | | Vital signs |
| not have serious | | Electrocardiograms (ECGs) |
| discontinuation symptoms | | Adverse Events |
| 11.9 Somnolence with A | | AEs leading to withdrawal |
| sustained release form of | | Serious discontinuation symptoms |
| quetiapine once daily is | | assessed by DESS (discontinuation scale) |
| generally mild, occurs early in | | AEs related to somnolence |
| treatment; is not persistent in | | Severity of somnolence reports |
| the majority of patients and is | | Time of AE somnolence reports |
| rarely a cause of withdrawal | | Withdrawals due to AE of somnolence |
| 11.10 Fasting Glucose and | | Change in weight from randomization to |
| Lipids not significantly | | each assessment |
| elevated | | Change in waist circumference from |
| 11.13 A sustained release form | | randomization to each assessment |
| of quetiapine once daily is | | Proportion of patient with a ≧7% increase |
| associated with a favourable | | from randomization weight. |
| weight profile | | |
| 11.14 A sustained release form | | |
| of quetiapine once daily has a | | |
| comparable incidence of | | |
| withdrawals due to adverse | | |
| events than escitalopram | | |
| 11.2 A sustained release form | For 11.2, 11.4, 11.6, and 11.12 | AEs (especially related to sexual |
| of quetiapine once daily is | To evaluate if sustained release | dysfunction, nausea, vomiting, EPS |
| associated with placebo levels | form of quetiapine is as safe and | including akathisia) |
| of sexual dysfunction | well-tolerated as placebo in the | Change from randomization to each |
| | treatment of patients with MDD | assessment in Sexual Functioning |
| 11.3 A sustained release form | For 11.3, 11.5, 11.7, 11.11 | Questionnaire (CSFQ) total score |
| of quetiapine once daily is | To evaluate if sustained release | Change in SAS and BARS from |
| associated with lower levels of | form of quetiapine is safer and | randomization to each assessment |
| sexual dysfunction than with | more well-tolerated than | MADRS item 10 score ≧4 at any time |
| escitalopram | escitalopram in the treatment of | after randomization or AE of |
| | patients with MDD | suicidality/suicidal ideation/suicide |
| 11.4 A sustained release form | | attempts/suicide completion |
| of quetiapine once daily is | | Analysis of suicidality according to FDA |
| associated with placebo levels | | guidance (tbc) |
| of nausea and vomiting | | |
| 11.5 A sustained release form | | |
| of quetiapine once daily is | | |
| associated with lower levels of | | |
| nausea and vomiting than | | |
| escitalopram | | |
| 11.6 A sustained release form | | |
| of quetiapine once daily is | | |
| associated with placebo levels | | |
| of EPS (including akathisia) | | |
| 11.7 A sustained release form | | |
| of quetiapine once daily is | | |
| associated with less EPS | | |
| (including akathisia) than | | |
| escitalopram | | |
| 11.11 A sustained release form | | |
| of quetiapine once daily is | | |
| associated with a lower | | |
| incidence of treatment | | |
| emergent suicidal ideation | | |
| compared with escitalopram | | |
| 11.12 A sustained release form | | |
| of quetiapine once daily is | | |
| associated with a lower | | |
| incidence of treatment | | |
| emergent suicidal ideation | | |
| compared with placebo | | |

The study comprises the following three periods:

1) Washout period (See Example 15, above)

2) Eight-Week double-blind, randomized, placebo controlled treatment period (Day 1 to Day 57)

Eligible patients will be randomized on Day 1 (Visit 2) to one of three treatment groups: sustained release form of quetiapine 150 mg/day, escitalopram 10mg/day or placebo. The likelihood of entering the placebo arm is 33%.

After 2 weeks of treatment, patients with inadequate response will receive a double dose of study medication. Inadequate response is defined by the following criteria: failure to decrease the initial MADRS score by 20% at week 2 from randomization. Patients responding to treatment will continue on initial dose.

Patients will be treated and assessed for 8 weeks according to schedule.

3) Two-week follow-up period including one week down-titration (Day 58 to Day 71)

All randomized patients will be asked to call in through an IVRS system to do an assessment of discontinuation-emergent signs and symptoms (DESS) at 1, 3, 5, 7 and 14 days after their final dose of study medication (Day 58, 60, 62, 64 and 71). Patients in the 300 mg/day sustained release form of quetiapine group and patients in the 20 mg/day escitalopram group will be down-titrated during the first follow-up week.

Number of Patients

About 450 patients (7% attrition rate), will be randomized to obtain 140 evaluable patients per treatment group (sustained release form of quetiapine, escitalopram and placebo arms) in a 1:1:1 randomization. An evaluable patient is a patient with at least one valid post-randomization MADRS assessment completed.

Investigational Products

The patients will be randomized to double blind treatment with either sustained release form of quetiapine 150 mg/day escitalopram 10 mg/day or placebo. After 2 weeks of treatment patients with inadequate response will be treated with double dose of the starting dose.

Tablets to be used in the study are: 50 and 300 mg quetiapine sustained release (SR) tablets; 10 mg escitalopram tablets; placebo tablets to match; and placebo capsules to match.

The sustained release form of quetiapine, escitalopram or placebo will be administered once daily at bedtime. All sustained release form of quetiapine patients will start on 50 mg/day, being uptitrated to 150 mg/day at day 3.

TABLE 29
Packaging and doses required for blinding

| Treatment group | Day 1-2 | Day 3-14 | Day 15-57 |
|---|---|---|---|
| Placebo | | | |
| Patients responding to | 3 placebo tablets 50 mg | 3 placebo tablets 50 mg | 3 placebo tablets 50 mg |
| treatment | 1 placebo tablet 300 mg | 1 placebo tablet 300 mg | 1 placebo tablet 300 mg |
| Patients with inadequate | 2 placebo capsules 10 mg | 2 placebo capsules 10 mg | 2 placebo capsules 10 mg |
| response assigned to double | | | |
| dose | | | |
| 150 mg sustained release form | | | |
| of quetiapine/day | | | |
| Patients responding to | 1 sustained release form | 3 sustained release form | 3 sustained release form |
| treatment (continue on 150 mg/ | of quetiapine tablets 50 mg | of quetiapine tablets 50 mg | of quetiapine tablets 50 mg |
| day) | 2 placebo tablets 50 mg | 1 placebo tablet 300 mg | 1 placebo tablet 300 mg |
| | 1 placebo tablet 300 mg | 2 placebo capsules 10 mg | 2 placebo capsules 10 mg |
| | 2 placebo capsules 10 mg | | |
| 150 mg sustained release form | | | |
| of quetiapine/day | | | |
| Patients with inadequate | 1 sustained release form | 3 sustained release form | 3 placebo tablets 50 mg |
| response assigned to double | of quetiapine tablets 50 mg | of quetiapine tablets 50 mg | 1 sustained release form |
| dose (300 mg/day) | 2 placebo tablets 50 mg | 1 placebo tablet 300 mg | of quetiapine tablet 300 mg |
| | 1 placebo tablet 300 mg | 2 placebo capsules 10 mg | 2 placebo capsules 10 mg |
| | 2 placebo capsules 10 mg | | |
| 10 mg escitalopram/day | | | |
| Patients responding to | 3 placebo tablets 50 mg | 3 placebo tablets 50 mg | 3 placebo tablets 50 mg |
| treatment (continue on 10 mg/ | 1 placebo tablet 300 mg | 1 placebo tablet 300 mg | 1 placebo tablet 300 mg |
| day) | 1 escitalopram capsule 10 mg | 1 escitalopram capsule 10 mg | 1 escitalopram capsule 10 mg |
| | 1 placebo capsule 10 mg | 1 placebo capsule 10 mg | 1 placebo capsule 10 mg |
| 10 mg escitalopram/day | | | |
| Patients with inadequate | 3 placebo tablets 50 mg | 3 placebo tablets 50 mg | 3 placebo tablets 50 mg |
| response assigned to double | 1 placebo tablet 300 mg | 1 placebo tablet 300 mg | 1 placebo tablet 300 mg |
| dose (20 mg/day) | 1 escitalopram capsule 10 mg | 1 escitalopram capsule 10 mg | 2 escitalopram capsules |
| | 1 placebo capsule 10 mg | 1 placebo capsule 10 mg | 10 mg |

After 8 weeks of treatment the patients should be down titrated according to the following schedule.

TABLE 30
Down titration of investigational product

| | sustained release | sustained release | | | |
|---|---|---|---|---|---|
| Treatment | form of quetiapine | form of quetiapine | Escitalopram | Escitalopram | |
| group | 150 mg/day | 300 mg/day | 10 mg/day | 20 mg/day | placebo |
| Day 58-64 | 3x 50 mg placebo | 3x 50 mg sustained | 3x 50 mg | 3x 50 mg | 3x 50 mg |
| | tablets | release form of | placebo tablets | placebo tablets | placebo tablets |
| | 1x 10 mg placebo | quetiapine tablets | 1x 10 mg | 1x 10 mg | 1x 10 mg |
| | capsule | 1x 10 mg placebo | placebo capsule | escitalopram | placebo capsule |
| | | capsule | | capsule | |
| Day 65-71 | No treatment | No treatment | No treatment | No treatment | No treatment |

Study Procedures

Eligibility for the study will be assessed at enrolment and randomization. The patients will be randomized to treatment groups at Day 1 after fulfilling all inclusion criteria and none of the exclusion criteria. All visits allow a visit window of ±2 days calculated from randomization.

After 2 weeks all patients will be assessed for response. Patients with inadequate response (defined by the following criteria: failure to decrease the initial MADRS score by 20% at week 2 from randomization), will received double dose of investigational product. Patients responding to treatment will continue on initial dose.

Statistical Analysis

Confirmatory Strategy

Primary objective: The null hypotheses is that there is no difference between the quetiapine treatment regimen and the placebo treatment regimen in change in MADRS total score from randomization to Week 8.

Secondary objective of particular interest: The null hypotheses is that there is no difference between the quetiapine treatment regimen and the placebo treatment regimen in change in Q-LES-Q total score from randomization to Week 8.

A step-wise sequential testing procedure will be used to handle multiple comparisons to ensure that the overall significance level of 0.05 is preserved. First the primary outcome variable change in MADRS total score from randomization to Week 8 will be tested. If the null hypothesis for this variable is rejected, then the variable change in Q-LES-Q total score from baseline to Week 8 will be tested.

Analysis Populations

The sustained release form of quetiapine treatment regimen and the placebo treatment regimen is defined as all patient initially randomized to sustained release form of quetiapine/placebo, regardless of they were classified as patients with inadequate response or patients with adequate response at the assessment of response at week 2. Thus, the patients initially randomized to sustained release form of quetiapine or placebo will for this hypothesis be regarded as one sustained release form of quetiapine and one placebo group, regardless the response at week 2. The sustained release form of quetiapine treatment regimen and the placebo treatment regimen will from now on be referred to as sustained release form of quetiapine and placebo, respectively.

The efficacy analyses will be based on the modified intention-to-treat population (Full Analysis Set). This population will include all randomized subjects, classified according to randomized treatment, who took study medication and who have a randomization MADRS assessment and at least 1 valid MADRS assessment after randomization.

The safety displays will be based on the safety population. This population includes all randomized subjects who took study medication, classified according to the treatment actually received.

Analysis of the Primary Outcome Variable

Primary Efficacy Analysis

The primary outcome variable, the change in MADRS total score from randomization to Week 8, will be analyzed using a mixed model analysis with MADRS total score at randomization as a covariate and including treatment as a fixed effect and centre as a random effect. The comparison of interest will be the difference between sustained release form of quetiapine and placebo.

Descriptive statistics including 95% confidence intervals around the estimate for the comparison of MADRS total score change from randomization between escitalopram and placebo will also be provided for assay sensitivity.

Secondary Efficacy Analysis of Primary Interest

The outcome variable, the change in Q-LES-Q total score from randomization to Week 8, will be analyzed using a mixed model analysis with Q-LES-Q total score at randomization as a covariate and including treatment as a fixed effect and centre as a random effect. The comparison of interest will be the difference between the sustained release form of quetiapine dose and placebo.

Descriptive statistics including 95% confidence intervals around the estimate for the comparison of Q-LES-Q total score change from randomization between escitalopram and placebo will also be provided

The sample size calculation in this study was done to demonstrate superior efficacy of sustained release form of quetiapine over placebo and were calculated with regard to the primary outcome variable, change in MADRS total score from randomization to week 8. The appropriate sample size was attained by assuming a clinically relevant difference of 3.5 units from placebo and a within patient variability (standard deviation) of 9 for the change in MADRS total score from randomization to Week 8. A power set to 90% yields a planned sample size of 140/arm, and 420 in total.

Assuming that 93% of all randomized patients are expected to be evaluable patients (to be included in MITT) without significant protocol violations or deviations, a total of about 450 randomized patients are required to obtain 140 evaluable patients per treatment group.

TABLE 31
Sample size calculations

| | As specified |
|---|---|
| Power | 90% |
| Difference to be detected | 3.5 |
| compared to placebo | |
| Standard deviation | 9 |
| Significance level | 0.05 |
| Sample size (evaluable) | 140/arm |

Note that the study is not powered for a formal comparison between escitalopram and placebo, only descriptive statistics will be provided for this comparison.
MDD Exclusion criteria (See “Exclusion criteria” in Example 14 above)
Restrictions in treatments (see “Restrictions” in Example 14 above)

### Example 17

### Treatment of GAD (50-300 mg/day)

The overall rationale for this study is to demonstrate the maintenance of effect and long-term safety of quetiapine fumarate (SEROQUEL®) in the treatment of patients with GAD.

A primary objective of this study is to evaluate the efficacy of quetiapine versus placebo with respect to risk of relapse of anxiety symptoms in patients with GAD (Table 29). The secondary objectives of the study are shown in Table 30.

TABLE 32
Primary Objective

| Claims to be | | |
|---|---|---|
| addressed | Primary Objective | Primary Outcome Variable |
| Quetiapine once | To evaluate the | Time to relapse: where relapse |
| daily efficacy | efficacy of quetiapine | is defined as a HAM-A total |
| is maintained | versus placebo | score = 15, or hospitalization |
| long term in | with respect to | due to GAD, or need to |
| patients with | risk of relapse of | initiate another medication to |
| GAD | anxiety symptoms | treat GAD, or attempted |
| | | suicide, or CGI-C score of = 4 |

TABLE 33
Secondary Objectives

| Claims to be addressed or | | |
|---|---|---|
| hypothesis to be tested | Secondary Objective | Secondary Outcome Variables |
| Quetiapine once daily efficacy | To evaluate the efficacy of | Relapse: where relapse is |
| is maintained long term in | quetiapine versus placebo in | defined as a HAM-A total score = 15, |
| patients with GAD | long-term treatment of patients | or hospitalization due to |
| | with GAD | GAD, or need to initiate another |
| | | medication to treat GAD, or |
| | | attempted suicide, or CGI-C |
| | | score of = 4 |
| | | Change from randomization in |
| | | HAM-A total score at Week 28 |
| Quetiapine once daily efficacy | To evaluate the efficacy of | Change from randomization in |
| is maintained long term in | quetiapine versus placebo in the | Clinical Global Impression |
| patients with GAD | long-term treatment of anxiety | Severity of Illness (CGI-S) |
| | symptoms in patients with GAD | score at Week 28. |
| | | CGI Global Improvement (CGI- |
| | | I) at each assessment after |
| | | randomization and Week 28. |
| | | Change from randomization in |
| | | HAM-A psychic cluster |
| | | (anxious mood, tension, fears, |
| | | insomnia, intellectual changes, |
| | | depressed mood, and behavior |
| | | at interview) at Week 28. |
| | | Change from randomization in |
| | | HAM-A somatic cluster |
| | | (somatic muscular, somatic |
| | | sensory, cardiovascular system, |
| | | respiratory system, |
| | | gastrointestinal system, |
| | | genitourinary system, |
| | | autonomic system) at Week 28. |
| Quetiapine once daily efficacy | To evaluate if the long term | Change from randomization in |
| is maintained long term in | treatment of quetiapine | Pittsburgh Sleep Scale Index |
| patients with GAD | maintains sleep quality in | (PSQI) score at each assessment |
| | patients with GAD, compared to | and Week 28. |
| | placebo | Change from randomization in |
| | | the MADRS sleep disturbance |
| | | factor (Item 4) at each |
| | | assessment and Week 28. |
| Quetiapine is more effective | To evaluate the efficacy of long- | Change from randomization in |
| than placebo in reducing | term treatment of quetiapine | Montgomery-Asberg |
| depressive symptoms in long- | versus placebo in the treatment | Depression Rating Scale |
| term treatment of patients with | of depressive symptoms in | (MADRS) total score at each |
| GAD | patients with GAD | assessment and Week 28. |
| Quetiapine once daily efficacy | To evaluate if quetiapine | Change from randomization in |
| is maintained long term in | maintains the quality of life of | Q-les-Q total score at each |
| patients with GAD | patients with GAD, compared to | assessment and Week 28. |
| | placebo during the long-term | Change from randomization in |
| | treatment. | Q-les-Q Item 16 (Overall life |
| | | satisfaction) at each assessment |
| | | and Week 28. |
| Quetiapine once daily efficacy | To evaluate if quetiapine | Change from randomization in |
| is maintained long term in | maintains patient satisfaction | Q-les-Q Item 15 (Satisfaction |
| patients with GAD | versus placebo in the long-term | with medication) at each |
| | treatment of patients with GAD | assessment and Week 28. |
| Quetiapine once daily efficacy | To evaluate the effect on | Change from randomization in |
| is maintained long term in | functional disability of long | the SDS total score at Week 28. |
| patients with GAD | term treatment of quetiapine | |
| | compared to placebo in patients | |
| | with GAD as assessed by the | |
| | change from randomization in | |
| | Sheehan Disability Scale (SDS) | |
| | total score | |
| Quetiapine once daily is well | To assess whether quetiapine is | Change from normal to |
| tolerated in patients with GAD | safe and well-tolerated in long- | Clinically Important in: Physical |
| long term | term treatment of patients with | Examinations; Laboratory |
| | GAD. | values (including |
| | To assess whether quetiapine is | glucose/lipids); Vital signs; |
| | as safe and well-tolerated as | Electrocardiograms (ECGs). |
| | placebo in long-term treatment | Adverse events: Adverse events |
| | of patients with GAD | related to nausea and vomiting |
| | | Adverse events related to EPS |
| | | (including akathisia) |
| | | Change in SAS/BARS, AIMS |
| | | scores from randomization to |
| | | Week 28 |
| | | Adverse events related to |
| | | discontinuation |
| | | Adverse events related to |
| | | somnolence |
| | | Severity of AEs related to |
| | | somnolence |
| | | Time to first instance of AE |
| | | somnolence |
| | | Withdrawals due to AE of |
| | | somnolence |
| | | Change in weight from |
| | | randomization to Week 28 |
| | | Change in waist circumference |
| | | from randomization to Week 28 |
| | | Change in weight of = 7% from |
| | | randomization to Week 28 |

This is a multicentre, randomized, parallel-group, double-blind, placebo-controlled, treatment-withdrawal, long-term study to evaluate the efficacy and safety of quetiapine for a minimum of 28 weeks of maintenance treatment in adult patients with GAD.

The study comprises the following three periods:

Enrolment Period

Enrolment will last for up to 28 days. To be eligible for the study, patients must have a documented clinical diagnosis of GAD confirmed according to DSM-IV (Diagnostic and Statistical Manual of Mental Disorders, Fourth Edition) criteria 300.02 as assessed by the MINI. Additionally, patients will need to meet the requirements for HAM-A, MADRS, CGI-S, COVI and RASKIN as outlined in the Inclusion Criteria to be enrolled in the study. Patients who meet all inclusion criteria and none of the exclusion criteria will enter the Open-Label Treatment Period at Visit 1.

Open-Label Treatment Period

During the Open-Label Treatment Period, patients will be administered open-label quetiapine for 8-12 weeks. The purpose of the Open-Label Treatment Period is to achieve stabilization before randomization, after acute treatment of anxiety.

The starting dose of quetiapine in the Open-Label Treatment Period will be 150 mg/day.

The prescribed quetiapine dosage may be adjusted to 50, 150 or 300 mg/day once daily to maximize efficacy and tolerability based upon the investigator's clinical experience

Visits will occur at Week 1, Week 2, Week 4, Week 6, Week 8, Week 10 and Week 12. Treatment with open-label quetiapine will continue until patients meet all the inclusion criteria and none of the exclusion criteria for randomization, for at least 8 weeks, but no longer than 12 weeks. In addition, patients will need to be on the same stable dose for two consecutive visits in order to qualify for randomization. To qualify for randomization, patients must have a 50% reduction in the HAM-A total score from screening and also a HAM-A total score of ≦10. Both criteria must be met at two consecutive visits while on the same stable dose in the Open-Label Treatment Period.

Randomized Treatment Period

Patients who meet all the inclusion criteria for randomization and none of the exclusion criteria for randomization will be randomized (at Visit 9) in a blinded fashion to quetiapine or matching placebo at the same stable dose level received during the Open-Label Treatment Period. The dosage can be adjusted as clinically indicated during the Randomized Treatment Period at the investigator's discretion.

All randomized patients will be assessed for discontinuation-emergent signs and symptoms (DESS) during the first week after randomization at Day 1, Day 3, Day 5, and Day 7, and at Day 14 during the second week. Adverse events and concomitant medications will be assessed after administration of the DESS checklist.

Patients will continue in the Randomized Treatment Period for a minimum of 28 weeks up to 80 weeks or until they meet the criteria for relapse as defined as a HAM-A total score of =15, or hospitalization for GAD, or the need to initiate another medication to treat GAD, or attempted suicide, or a CGI-C score of =4.

Patients who reach a HAM-A total score of =15 will be required to return to the study site the following week to repeat the HAM-A assessment. Patients must have a HAM-A total score of =15 at both assessments to be qualified as a relapse and discontinued from the study. However, if the patient discontinues from the study after first assessment of HAM-A total score of =15, then patient will also be qualified as a relapse.

The study physician must document CGI-C=4 at a study visit or a phone call interview within 1 week of a missed study visit in order for patient to be qualified as a relapse. If patient is lost to follow up and later found to have been hospitalized due to GAD within 30 days of missed visit, then patient qualifies as a relapse. Patients who are prescribed a medication by a physician to treat GAD will qualify as a relapse. Additionally, patients who self-medicate with exclusionary medications to treat GAD for 1 week or greater will be qualified as a relapse and discontinued.

Patients may also be discontinued from the study due to lack of efficacy, adverse event, patient lost to follow-up, protocol noncompliance, or informed consent withdrawn.

The study will be terminated when the last patient has completed 28 weeks of treatment or until they meet the criteria for relapse as defined above. The final number of randomized patients may change during the study based on observed event rates.

Study Population

Patients will be male or female, 18 to 70 years of age, with a DSM-IV diagnosis of GAD according to DSM-IV (Diagnostic and Statistical Manual of Mental Disorders, Fourth Edition) criteria 300.02 as assessed by the MINI.

Patients are required to have a HAM-A total score of =20 with both Item 1 and Item 2 scores =2 at the Screening Visit. Patients are required to have CGI-S score=4 at the Screening Visit. Additionally, patients are required to have a total COVI score=8 and greater than the total RASKIN item score at the Screening Visit with all individual RASKIN item scores=3.

Patients suffering from depressive symptoms, defined as having a MADRS total score≧17 at the Screening Visit will be excluded from participation in this study.

In order to be randomized to the blinded treatment phase of this study, patients are required to have a total HAM-A score reduction of =50% from the baseline (screening) and a total HAM-A score of =10 at two consecutive study visits during the Open-Label Treatment Period while on the same stable dose of quetiapine.

Number of Patients

It is estimated that 352 subjects will need to be enrolled in the open-label treatment phase to obtain 176 eligible patients for the randomization treatment phase. An evaluable subject will be defined as a subject who has used study medication in the Randomized Treatment Period.

Study Duration

This study will consist of 8-12 weeks of open-label treatment followed by a minimum of 28-week randomized treatment period.

Drug Formulation; Doses and Dosing Regimen

In the Open-Label Treatment Period, all patients will start on a quetiapine 150 mg/day. The quetiapine will be administered once daily at bedtime. The dosage of quetiapine can be increased to 300 mg/day or decreased to 50 mg/day based upon the clinical judgment of the investigator. All patients will need to be maintained at the same stable dose of quetiapine for two consecutive visits (4 weeks) prior to randomization.

Eligible patients will be randomized to a double blind treatment with quetiapine or matching placebo at the same stable dose received during the Open-Label Treatment Period.

Starting at Visit 9 (randomization), open-label quetiapine tablets will be replaced with tablets of blinded quetiapine or matching placebo tablets. Open-label treatment will end abruptly and replaced with double blind treatment.

Investigators are encouraged to use investigational products (randomized treatments) to treat anxiety, sleep, and other symptoms before prescribing the restricted sleep medications.

Tablets to be used in the study are: 50 and 300 quetiapine sustained release (SR) tablets and placebo tablets to match.

Statistical Analysis

Confirmatory Strategy

Primary claim: The null hypothesis is that there is no difference in relative risk of relapse between quetiapine and placebo treatment groups. Relapse is defined as: a HAM-A total score of =15, or hospitalization for GAD, or the need to initiate another medication to treat GAD, or attempted suicide, or a CGI-C score of =4.

The null hypothesis that there is no difference between quetiapine and placebo with respect to the secondary outcome, change from randomization in Q-LES-Q total score. The strategy is to control the overall experiment type I error while including this additional comparison with the primary comparison.

Multiple Comparisons Procedure:

In order to take account of these 2 comparisons, a stepwise sequential procedure will be used for the 2 analyses in the confirmatory part of this study to ensure an overall experiment type I error of 0.05. These outcomes will be tested sequentially in the following order: time to relapse followed by Q-LES-Q. Q-LES-Q will only be formally tested if time to relapse is statistically significant using a 2-sided test and alpha=0.05.

Analysis of the Primary Outcome Variable

The primary efficacy outcome variable will be analyzed using a Cox proportional hazards model. An estimate of the hazard ratio for relapse between treatment groups, with 95% confidence intervals will be provided. A two-sided test of the null hypothesis that the hazard ratio is equal to unity will be performed. For those patients without an event (i.e. relapse), the time to relapse will be censored when a patient discontinues from or completes the study. The time of censoring will be the date of the patient's final assessment.

The study is powered to show that quetiapine is different from placebo with respect to risk of relapse, primary efficacy outcome variable.

TABLE 34
Sample Size Calculation

| | |
|---|---|
| Power | 90% |
| Hazard ratio | .375 |
| % Relapse in Quetiapine | .109 |
| % Relapse in Placebo | .399 |
| % Relapse Overall | .254 |
| Significance level | 5% two-sided |
| Number of evaluable per group in | 87 (174) |
| Randomized treatment phase (total) | |
| Number randomized per group in | 88 (176) |
| Randomized treatment phase (total) | |
| Sample size per treatment arm needed | 176 (352) |
| at Open-label portion (total) | |

An evaluable subject will be defined as a subject who has used study medication in the randomized treatment phase.

The sample size estimate was based on another randomized withdrawal design for Paroxetine. In this article, placebo showed 39.9% relapse and paroxetine showed 10.9%. The definition of relapse in this article was an increase in CGI-S score of at least 2 points to a score of >=4, or withdrawal resulting from lack of efficacy. Also, in this article the percent remission was 30% for placebo and 70% for paroxetine. Using incidence of remission, the current sample size would allow us to detect a difference in 25% points at 90% power assuming the overall remission rates is approximately 60%. Another long-term study in Venlafaxine XR showed a difference of 4.18 points in HAM-A total score with s.d.=7.5. The current sample size would be overpowered to show this difference. Another long-term study in Venlafaxine also noted a 5.4 change in HAM-A total score compared to placebo at 6 months.

The drop-out of 50% in the open label portion has been estimated and will be monitored throughout the study in order to obtain the number evaluable for the randomized treatment phase.

Study-Specific Inclusion/Exclusion Criteria, Restrictions and Rationale

TABLE 35
Inclusion Criteria

| Inclusion Criteria | Rationale |
|---|---|
| Provision of written informed consent before | Mandatory according |
| initiation of any study related procedures | to GCP |
| Male or female aged 18 to 70 years | To include adult |
| | subjects |
| A documented clinical diagnosis of GAD | Selection of patients |
| according to DSM-IV (Diagnostic and Statistical | whose anxiety is not |
| Manual of Mental Disorders, Fourth Edition) | part of another |
| criteria 300.02 as assessed by the MINI (Mini- | disorder |
| International Neuropsychiatric Interview) | |
| A HAM-A total score = 20 with Item 1 (anxious | To ensure that patients |
| mood) and Item 2 (tension) scores = 2 at | are sufficiently acutely |
| enrolment | ill |
| A CGI-S score = 4 at enrolment | To ensure that patients |
| | are sufficiently acutely |
| | ill |
| A COVI total score = 8 and greater than the | To avoid selection of |
| RASKIN total score at enrolment and all | patients with |
| RASKIN item scores = 3 at enrolment | depression |
| Female patients of childbearing potential must | To ensure safety |
| have a negative serum pregnancy test at | |
| enrolment and be willing to use a reliable | |
| method of birth control (i.e., barrier method, | |
| oral contraceptive, implant, dermal contraception, | |
| long-term injectable contraceptive, intrauterine | |
| device, or tubal ligation) during the study, as | |
| judged by the investigator | |
| Be able to understand and comply with the | To ensure protocol |
| requirements of the study, as judged by the | compliance and |
| investigator | generate evaluable |
| | data |

TABLE 36
Exclusion Criteria: Including but not limited to the following:

| Exclusion Criteria | Rationale |
|---|---|
| Patients with a current DSM-IV Axis I disorder | To exclude other diagnoses which may confound |
| other than GAD (with or without simple phobia) | results |
| within 6 months of Day 0, Visit 2 | |
| The presence or history of any psychotic | Not part of the target population, would |
| disorder | potentially confound the results |
| The presence of any DSM-IV axis II disorder | Not part of the target population |
| that is likely to interfere with the patient's ability | |
| to participate in the study as judged by the | |
| investigator | |
| Patients suffering from depressive symptoms, | Not part of the target population |
| defined as having a MADRS total score ≧17 at | |
| enrolment | |
| Patients who, in the investigator's judgment, | To ensure safety |
| pose a current serious suicidal or homicidal risk | |
| or have made a suicide attempt | |
| Evidence of clinically relevant disease (e.g., | Could potentially confound study results |
| renal or hepatic impairment, significant coronary | To ensure safety |
| artery disease, cerebrovascular disease, viral | |
| hepatitis B or C, acquired immunodeficiency | |
| syndrome [AIDS], or cancer), or a clinical | |
| finding that is unstable or, in the opinion of the | |
| investigator, would either be negatively affected | |
| by the study medication or would affect the | |
| study medication | |
| History of seizure disorder, except febrile | To ensure safety |
| convulsions | |
| Substance or alcohol abuse or dependence, as | To ensure protocol compliance and generate |
| defined in DSM-IV criteria, within 6 months | evaluable data |
| prior to Day 0, Visit 2 (except dependence in full | |
| remission, caffeine dependence, or nicotine | |
| dependence). Patients with a positive urine | |
| toxicology screen for a drug of abuse will be | |
| excluded with the exception of patients testing | |
| positive for cannabinoids. For patients testing | |
| positive for cannabinoids at enrolment to be | |
| entered, they must not meet abuse or dependence | |
| criteria, and in the judgment of the investigator | |
| will not use cannabinoids or other illegal or non | |
| prescribed drugs during the study | |
| Use of antipsychotic medication within 28 days | Could potentially confound study results |
| prior to Day 0, Visit 2. | |
| Receipt of electroconvulsive therapy (ECT) | Could potentially confound study results |
| within 28 days prior to Day 0, Visit 2. | |
| Patients who in the investigators opinion will | To prevent new therapies being introduced during |
| require psychotherapy (other than supportive | study treatment period |
| psychotherapy) during the study, unless | |
| psychotherapy has been ongoing for a minimum | |
| of 3 months prior to Day 0, Visit 2 | |
| Use of benzodiazepines, antidepressants, MOA | Could potentially confound study results |
| inhibitors and mood stabilizers within 14 days | |
| prior to Day 0, Visit 2. | |
| Use of benzodiazepines (maximum allowable | Could potentially confound study results |
| dose of 10-mg equivalent of diazepam) as a | |
| single dose more than 3 times per week in the | |
| period 14 to 28 days prior to Day 0, Visit 2 (ie, | |
| Day −15 to Day −28) | |
| Use of hypnotics (maximum allowable dose of | Could potentially confound study results |
| 10 mg zolpidem tartrate, 1 gram chloral hydrate) | |
| at bedtime more than 3 times per week in the 28 | |
| days prior to Day 0, Visit 2. | |
| Administration of a depot antipsychotic injection | Could potentially confound study results |
| within 2 dosing intervals prior to Day 0, Visit 2. | |
| Use of potent cytochrome P450 (CYP) 3A4 | To avoid drug interaction |
| inducers (e.g., barbiturates, carbamazepine, | |
| glucocorticoids, phenytoin, rifampin, rifabutin, | |
| thioridazine, and St John's Wort) in the 14 days | |
| preceding Day 0, Visit 2. | |
| Use of potent CYP 3A4 inhibitors (e.g., | To avoid drug interaction |
| macrolide antibiotics [clarithromycin, | |
| fluvoxamine, nefazodone, erythromycin, | |
| troleandomycin]; azolantifungals [fluconazole, | |
| itraconazole, ketoconazole (except for topical | |
| use)]; protease inhibitors [indinavir, nelfinavir, | |
| ritonavir, saquinavir]) in the 14 days preceding | |
| Day 0, Visit 2. | |

Criteria for Entering Randomized Treatment Period

TABLE 37
Inclusion Criteria

| Inclusion criteria | Rationale |
|---|---|
| Patient has been prescribed a dose of quetiapine | Quetiapine dose to be |
| within the range of 50, 150 or 300 mg/day for at | used in the |
| least 8 weeks | Randomized |
| | Treatment Period |
| Patients who responded with = 50% reduction in | Required for |
| HAM-A total score from screening and a HAM- | stabilisation |
| A total score of = 10. Both criteria must be met | Establishes remission |
| at two consecutive visits at the same stable dose | |

TABLE 38
Exclusion Criteria

| Exclusion criteria | Rationale |
|---|---|
| MADRS = 17 during the Open-Label | Not part of the target |
| Treatment Period | population |
| Hospitalization due to GAD symptoms during | Indicates patient is not |
| the Open-Label Treatment Period | stable. Hospitalization |
| | is part of event criteria |
| | during Randomized |
| | Treatment Period. |
| The need to initiate another medication to treat | |
| GAD during the Open-Label Treatment Period | |
| Attempt to commit suicide or homicide during | Indicates patient is |
| the Open-Label Treatment Period | not stable. |

Restrictions

TABLE 39
Restrictions in treatments during the study

| Prohibited Treatments | Rationale |
|---|---|
| Use of drugs that induce or inhibit the hepatic | Potentially confounds the results |
| metabolizing cytoctrome 3A4 enzymes [e.g. | To ensure safety |
| inducers: carbamazepine, phenytoin, | |
| barbiturates, rifampin, rifabutin, glucocorticoids, | |
| thioridazine and St John's wort, and inhibitors: | |
| ketoconazole (except for topical use), | |
| itraconazole, fluconazole, erythromycin, | |
| clarithromycin, fluvoxamine, nefazodone, | |
| troleandomycin, indinavir, nelfinavir, ritonavir, | |
| and saquinavir]. | |
| Use of any psychoactive drugs including | Potentially confounds the results |
| antidepressant, anxiolytic, hypnotic, mood | |
| stabilizing, antipsychotic, and sedative | |
| medications other than restricted | |
| Prophylactic use of anticholinergics is | Potentially confounds the results |
| prohibited. | |
| Electroconvulsive therapy (ECT). | Potentially confounds the results |
| Abuse according to the DSM-IV criteria of | Potentially confounds the results |
| Alcohol, Opiates, amphetamine, barbiturate, | |
| cocaine, cannabis, or hallucinogen throughout | |
| the study | |
| Restricted Treatments | Rationale |
| Anticholinergics can be used to treat | Need to allow anticholinergics for treatment of |
| extrapyramidal symptoms. | EPS but not prophylactic as it could potentially |
| | mask EPS. |
| Psychotherapy is only allowed if it has been | Potentially confounds the results |
| ongoing since at least 3 months prior to | |
| screening | |
| From enrolment until Day 14 of Open-Label | Need to allow hypnotics at reasonable doses. |
| Treatment Period only, one of the following can | Differences in treatment traditions and availability |
| be used for insomnia, maximum 2 times per | of products between countries require alternative |
| week, up to the specified dosage per night; | products. |
| hypnotic use not allowed on the night prior to | |
| conducting study assessments: | |
| zolpidem tartrate 10 mg | |
| chloral hydrate, 1 g. | |

### Example 18

### Treatment of GAD with 50, 150, and 300 mg/Day

The overall rationale for this study is to demonstrate that quetiapine fumarate (SEROQUEL ) is efficacious and safe in the acute treatment of patients with GAD. This trial will serve as one of three studies to investigate the short-term efficacy and safety of quetiapine in GAD.

The primary objective is to evaluate the efficacy of quetiapine fumarate (SEROQUEL®) compared to placebo in the treatment of anxiety symptoms in patients with GAD (Table 37). The secondary objectives of the study are listed in Table 38.

This is an 8-week, 4-arm, randomized, parallel-group, double-blind, placebo-controlled Phase III study of the efficacy and safety of quetiapine fumarate (SEROQUEL®) 50, 150, 300 mg/day compared with placebo in the treatment of GAD. The study is comprised of three periods, the Screening Period, the Treatment Period and the Post-Treatment Period.

TABLE 40
Primary Objective

| | | Primary Outcome |
|---|---|---|
| Claims to be addressed | Primary Objective | Variable |
| Quetiapine is more | To evaluate the efficacy of | Change from |
| effective than placebo | quetiapine versus placebo | randomization in the |
| in GAD as measured | in the treatment of | Hamilton Anxiety |
| by total HAM-A score | anxiety symptoms | Scale (HAM-A) |
| | in patients with GAD | total score at Day 57 |

TABLE 41
Secondary Objectives

| Claims to be addressed or | | |
|---|---|---|
| hypothesis to be tested | Secondary Objective | Secondary Outcome Variables |
| Quetiapine demonstrates an | To evaluate the early | Change from randomization in the |
| anti-anxiety effect by Day 8 | efficacy of quetiapine in | HAM-A total score at Day 8 |
| | the treatment of anxiety | Change from randomization in |
| | symptoms in patients with | HAM-A psychic cluster (anxious |
| | GAD | mood, tension, fears, insomnia, |
| | | intellectual changes, depressed |
| | | mood, and behavior at interview) at |
| | | Day 8 |
| | | Change from randomization in |
| | | HAM-A somatic cluster (somatic |
| | | muscular, somatic sensory, |
| | | cardiovascular system, respiratory |
| | | system, gastrointestinal system, |
| | | genitourinary system, autonomic |
| | | system) at Day 8 |
| | | Change from randomization in |
| | | Clinical Global Impression Severity |
| | | of Illness (CGI-S) score at Day 8 |
| Quetiapine has a greater | To evaluate the early | HAM-A Response (decrease from |
| response rate at Day 8 than | efficacy of quetiapine | randomization total score of ≧50%) |
| placebo | versus placebo by | at Day 8 |
| | evaluating the response rate | |
| | in the treatment of anxiety | |
| | symptoms in patients with | |
| | GAD | |
| Quetiapine is more effective | To evaluate the efficacy of | Change from randomization in CGI- |
| than placebo in GAD across | quetiapine in the treatment | S score at Day 57 |
| anxiety symptoms | of anxiety symptoms in | CGI Global Improvement (CGI-I) at |
| | patients with GAD | Day 57 (much or very much |
| | | improved) |
| | | Change from randomization in |
| | | HAM-A psychic cluster (anxious |
| | | mood, tension, fears, insomnia, |
| | | intellectual changes, depressed |
| | | mood, and behavior at interview) at |
| | | Day 57 |
| | | Change from randomization in |
| | | HAMA somatic cluster (somatic |
| | | muscular, somatic sensory, |
| | | cardiovascular system, respiratory |
| | | system, gastrointestinal system, |
| | | genitourinary system, autonomic |
| | | system) at Day 57 |
| Quetiapine has a greater | To evaluate the efficacy of | HAM-A Response (decrease from |
| response rate than placebo in | quetiapine versus placebo | randomization total score of ≧50%) |
| patients with GAD | by evaluating the response | at Day 57 |
| | rate in the treatment of | |
| | anxiety symptoms in | |
| | patients with GAD | |
| Quetiapine is better than | To evaluate the efficacy of | HAM-A Remission (HAM-A total |
| placebo at achieving remission | quetiapine versus placebo | score ≦7) at Day 57 |
| in patients with GAD | by evaluating the remission | |
| | rate in the treatment of | |
| | anxiety symptoms in | |
| | patients with GAD | |
| Quetiapine is more effective | To evaluate the efficacy of | Change from randomization in |
| than placebo in reducing | quetiapine versus placebo | Montgomery-Asberg Depression |
| depressive symptoms in GAD | in the treatment of | Rating Scale (MADRS) total score at |
| | depressive symptoms in | Day 57 |
| | patients with GAD | |
| Quetiapine is superior to | To evaluate the efficacy of | Change from randomization in |
| placebo in improving sleep | quetiapine versus placebo | Pittsburgh Sleep Scale Index (PSQI) |
| quality in patients with GAD | in improving sleep quality | score at Day 57 |
| | in patients with GAD | Change from randomization in the |
| | | MADRS sleep disturbance factor |
| | | (Item 4) at Day 57 |
| Quetiapine is more effective | To evaluate the effect of | Change from randomization in the Q- |
| than placebo in improving the | quetiapine versus placebo | les-Q total score at Day 57 |
| quality of life of patients with | on the quality of life of | Change from randomization in the Q- |
| GAD | patients with GAD | les-Q Item 16 (Overall life |
| | | satisfaction) at Day 57 |
| Quetiapine is more effective | To evaluate if quetiapine | Change from randomization in Q-les- |
| than placebo in improving | improves patient | Q Item 15 (Satisfaction with |
| patient satisfaction in patients | satisfaction versus placebo | medication) at Day 57 |
| with GAD | in patients with GAD | |
| Quetiapine once daily up to | To assess the safety and | Change from normal to Clinically |
| 300 mg/day is safe and well | tolerability of quetiapine in | Important in: Physical Examinations; |
| tolerated in patients with GAD | patients with GAD | Laboratory values (including |
| Quetiapine is associated with | | glucose/lipids); Vital signs; |
| placebo levels of nausea and | | Electrocardiograms (ECGs). |
| vomiting | | Adverse events of nausea or |
| Quetiapine is associated with | | vomiting |
| placebo levels of EPS | | Adverse events related to EPS |
| (including akathisia) | | (including akathisia) |
| Quetiapine does not have | | Change from randomization in the |
| serious discontinuation | | SAS and BARS scores at Day 57 |
| symptoms | | Adverse events related to |
| Somnolence with quetiapine is | | discontinuation |
| generally mild, occurs early in | | Change in Discontinuation-Emergent |
| treatment; is not persistent in | | Signs and Symptoms total score at |
| the majority of patients and is | | Day 64 and Day 71 |
| rarely a cause of withdrawal as | | Severity of AEs related to |
| compared to placebo | | somnolence |
| Quetiapine is associated with a | | Adverse events related to |
| favorable weight profile in | | somnolence |
| patients with GAD | | Time of first instance of somnolence |
| | | Withdrawals due to AE of |
| | | somnolence |
| | | Change in weight from |
| | | randomization to Day 57 |
| | | Change in waist circumference from |
| | | randomization to Day 57 |
| | | Clinically significant weight gain |
| | | (patients with ≧7% increase from |
| | | randomization weight at Day 57) |

Screening Period

Eligibility for the study will be assessed at the Screening Visit. The Screening Period can extend up to 14 days prior to the Randomization Visit, at Day 1. Patients will undergo procedures and assessments prior to randomization.

Treatment Period

Eligible patients will be randomized on Day 1 (Visit 2) to one of four treatment groups: quetiapine 50 mg/day, quetiapine 150 mg/day, quetiapine 300 mg/day, or placebo. All patients 10 will follow a dose titration scheme to reach the randomized dose level. The dose titration scheme is as follows: Day 1 and Day 2-50 mg quetiapine, Day 3 and Day 4-150 mg quetiapine and Day 5 and up—300 mg quetiapine. Patients will be dosed to their appropriate level in a blinded fashion according to their treatment arm. Patients will receive 8 weeks of treatment from Day 1 to Day 56 and will undergo the procedures and assessments.

Post-Treatment Period

All randomized patients will be asked to call in to an Interactive Voice Response System (IVRS) for assessment of discontinuation-emergent signs and symptoms (DESS). Baseline DESS will be collected at the final study visit, Day 57. Patients will then call in to the IVRS at Day 1, Day 3, Day 5, Day 7 and Day 14 after the final study visit, Day 57. The baseline DESS assessment will be performed at the final study visit (Day 57) via IVRS at the physician's office. The rest of the assessments will be conducted at home.

Patients randomized to 300 mg quetiapine will be dose reduced to 150 mg quetiapine from Day 57 to Day 64. All other patients will receive matching and blinded placebo during this time. All patients will return for a Day 64 visit. Patients should perform the DESS at home on this day and are not required to complete DESS assessment at this office visit. There will be no study drug administered after Day 64.

Study Population

Patients will be male or female, 18 to 65 years of age, with a DSM-IV diagnosis of GAD according to DSM-IV (Diagnostic and Statistical Manual of Mental Disorders, Fourth Edition) criteria 300.02 as assessed by the MINI.

Patients are required to have a HAM-A (conducted by the Structured Interview Guide for HAM-A [SIGH-A]) total score of ≧20 with both Item 1 and Item 2 scores≧2 at both the Screening and Randomization Visits. Patients are required to have CGI-S score≧4 and MADRS score<17 at both the Screening and Randomization Visits. Additionally, patients are required to have a total COVI score≧8 and greater than the total RASKIN item score at both the Screening and Randomization Visits with all individual RASKIN item scores≦3 at both screening and randomization.

Number of Patients

About 876 patients will be randomized to obtain 812 evaluable patients in total, assuming that 7% of the patients will be inevaluable. Recruitment of patients will be stopped when it is determined that 812 randomized patients are evaluable. An evaluable patient will be defined as a patient who has used study medication, has a HAM-A total score at randomization and at least one HAM-A total score post-randomization.

Study Duration

Eligible patients will receive 56 days of randomized treatment. The Treatment Period will be followed by a 2-week Post-Treatment Period to measure discontinuation-emergent signs and symptoms (DESS). Patients randomized to the quetiapine 300 mg treatment arm will be down titrated during the Post-Treatment Period according to Table 32. 11 treatment arms except for the 300 mg treatment arm will receive placebo during the first week of the Post-Treatment Period. There will be no study medication dispensed during the second week of the Post-Treatment Period.

Drug Formulation; Doses and Dosing Regimen

The sustained release (SR) formulation of quetiapine (or matching placebo) will be administered once daily, in the evening, from Day 1 with doses escalating to reach each target dose according to Table 42.

TABLE 42
Dose Escalation of Investigational Products

| | Treatment group | | | |
|---|---|---|---|---|
| | Quetiapine | Quetiapine | Quetiapine | |
| Day | 50 mg/day | 150 mg/day | 300 mg/day | Placebo |
| 1 and 2 | 50 mg | 50 mg | 50 mg | Placebo |
| 3 and 4 | 50 mg | 150 mg | 150 mg | Placebo |
| 5 and up | 50 mg | 150 mg | 300 mg | Placebo |

Additionally, patients will be dose reduced during the Post-Treatment Period in a blinded fashion according to Table 43. Patients will not receive investigational product during the second week of the Post-Treatment Period (Day 64 to Day 71).

TABLE 43
Dose Reduction of Investigational Products

| | Treatment group | | | |
|---|---|---|---|---|
| | Quetiapine | Quetiapine | Quetiapine | |
| Day | 50 mg/day | 150 mg/day | 300 mg/day | Placebo |
| 56 | 50 mg | 150 mg | 300 mg | Placebo |
| 57 to 63 | Placebo | Placebo | 150 mg | Placebo |
| 64 to 70 | No Inv. Prod. | No Inv. | No Inv. Prod. | No Inv. Prod. |
| | | Prod. | | |

Study Procedures

Eligibility for the study will be assessed at the Screening and Randomization Visits. Eligible patients will be randomized to treatment groups at Day 1 after fulfilling all inclusion criteria and none of the exclusion criteria.

Statistical Analysis for Primary Endpoint

Confirmatory Strategy

Primary claim: The null hypotheses are that there are no differences between quetiapine (each dose) and placebo with respect to the primary outcome, change from randomization in HAM-A total score at Day 57. There will be 3 primary comparisons tested: each quetiapine dose compared to placebo. The overall experiment type I error rate will be set to 0.05.

Key regulatory claim: Q-LES-Q total score has been identified as a key additional regulatory claim. Specifically this would include the null hypotheses that there are no differences between quetiapine (each dose) and placebo with respect to the secondary outcome, change from randomization in Q-LES-Q total score at Day 57. Again, there will be 3 key secondary comparisons tested: each quetiapine dose compared to placebo. The strategy is to control the overall experiment type I error while including these 3 additional comparisons with the 3 primary comparisons.

Multiple Comparisons Procedure:

In order to take account of these 6 comparisons, a parallel gatekeeper approach will be used. The primary hypotheses serve as a gatekeeper in the sense that the key secondary hypotheses of interest (Q-LES-Q) will be tested only after the primary analysis has yielded a statistically significant result. Using a gatekeeping strategy for testing the primary and secondary hypotheses will preserve the overall experiment type I error rate at 0.05. Weights will be applied equally within the primary claim hypotheses (i.e. HAM-A comparisons) and set at 0.333. Similarly, weights will be applied equally within the key secondary claim hypotheses (i.e. Q-LES-Q comparisons) and set at 0.333.

Analysis Populations

The efficacy analyses will be based on the modified intention-to-treat population (Full Analysis Set). This population will include all randomized subjects, classified according to randomized treatment, who took study medication and who have a randomization HAM-A total score assessments and at least one HAM-A total score post-randomization.

The safety displays will be based on the safety population. This population includes all randomized subjects who took study medication, classified according to the treatment actually received.

Analysis of the Primary Outcome Variable

The primary efficacy outcome variable will be analyzed using an analysis of covariance (ANCOVA) model including the randomization HAM-A total score, centre, and treatment group as variables in the analysis.

Missing Data

Last observation carried forward (LOCF) is defined in the following way: the latest post randomization valid value before the missing data will be used. Randomization values will not be carried forward.

LOCF methodology will be used for the primary outcome variable.

The study is powered to show that either dose of quetiapine 50 mg, 150 mg or 300 mg is different from placebo with respect to the primary efficacy outcome variable, change from randomization in HAM-A total score at Day 57 (Table 41).

TABLE 44
Sample size calculation

| | |
|---|---|
| Power | 90% |
| Difference to be detected | 2.75 |
| Standard deviation | 7.5 |
| Significance level (specify | 5% overall; Bonferroni for each |
| one- or two-tailed) | comparison at 0.0166 |
| Total sample size (each | 812 (203) |
| group) | |

It is estimated that 876 subjects will need to be randomized to obtain 812 evaluable patients (using an assumption of a 7% unevaluable rate). An evaluable subject will be defined as a subject who has used study medication, has a HAM-A total score at randomization and at least one HAM-A total score post randomization).

The sample size estimate was based on other 8-week GAD trials. Maximum treatment differences observed in various Effexor XR trials (NDA 20-699/S-001) ranged from 2.3 to 2.9 points. Similar results were noted in a published paroxetine trial. Standard deviations and non-evaluable rates from these trials ranged from 7.2 to 8.8 points and 1% to 12% respectively.

Study-Specific Inclusion/Exclusion Criteria, Restrictions and Rationale

TABLE 45
Inclusion Criteria

| Inclusion Criteria | Rationale |
|---|---|
| Male or female aged 18 to 65 years | To include adult |
| | subjects |
| A documented clinical diagnosis of GAD | Selection of patients |
| according to DSM-IV (Diagnostic and Statistical | whose anxiety is not |
| Manual of Mental Disorders, Fourth Edition) | part of another |
| criteria 300.02 as assessed by the MINI (Mini- | disorder |
| International Neuropsychiatric Interview) | |
| A HAMA total score ≧20 with Item 1 (anxious | To ensure that patients |
| mood) and Item 2 (tension) scores ≧2 at both | are sufficiently acutely |
| screening and randomization | ill |
| A CGI-S score ≧4 at both screening and | To ensure that patients |
| randomization | are sufficiently acutely |
| | ill |
| A COVI total score ≧8 and greater than the | To avoid selection of |
| RASKIN total score at both screening and at | patients with |
| randomization and all RASKIN item scores ≦3 | depression |
| at screening and randomization | |

TABLE 46
Exclusion Criteria

| Exclusion Criteria | Rationale |
|---|---|
| Patients with a current DSM-IV Axis I disorder | To exclude other diagnoses which may confound |
| other than GAD (with or without simple phobia) | results |
| within 6 months of screening | |
| The presence or history of any psychotic | Not part of the target population, would |
| disorder | potentially confound the results |
| The presence of any DSM-IV axis II disorder | Not part of the target population |
| that is likely to interfere with the patient's ability | |
| to participate in the study as judged by the | |
| investigator | |
| Patients suffering from depressive symptoms, | Not part of the target population |
| defined as having a MADRS total score ≧17 at | |
| either screening or randomization | |
| Patients who, in the investigator's judgment, | To ensure safety |
| pose a current serious suicidal or homicidal risk | |
| or have made a suicide attempt | |
| Evidence of clinically relevant disease (e.g., | Could potentially confound study results |
| renal or hepatic impairment, significant coronary | To ensure safety |
| artery disease, cerebrovascular disease, viral | |
| hepatitis B or C, acquired immunodeficiency | |
| syndrome [AIDS], or cancer), or a clinical | |
| finding that is unstable or, in the opinion of the | |
| investigator, would either be negatively affected | |
| by the study medication or would affect the | |
| study medication | |
| History of seizure disorder, except febrile | To ensure safety |
| convulsions | |
| Substance or alcohol abuse or dependence, as | To ensure protocol compliance and generate |
| defined in DSM-IV criteria, within 6 months | evaluable data |
| prior to screening (except dependence in full | |
| remission, caffeine dependence, or nicotine | |
| dependence). Patients with a positive urine | |
| toxicology screen for a drug of abuse will be | |
| excluded with the exception of patients testing | |
| positive for cannabinoids. For patients testing | |
| positive for cannabinoids at screening to be | |
| enrolled, they must not meet abuse or | |
| dependence criteria, and in the judgment of the | |
| investigator will not use cannabinoids or other | |
| illegal or non prescribed drugs during the study | |
| Use of antipsychotic medication within 28 days | Could potentially confound study results |
| prior to randomization. | |
| Receipt of electroconvulsive therapy (ECT) | Could potentially confound study results |
| within 28 days prior to randomization. | |
| Patients who in the investigators opinion will | To prevent new therapies being introduced during |
| require psychotherapy (other than supportive | study treatment period |
| psychotherapy) during the study period, unless | |
| psychotherapy has been ongoing for a minimum | |
| of 3 months prior to randomization | |
| Use of benzodiazepines, antidepressants, MAO | Could potentially confound study results |
| inhibitors and mood stabilizers within 14 days | |
| prior to randomization. | |
| Use of benzodiazepines (maximum allowable | Could potentially confound study results |
| dose of 10-mg equivalent of diazepam) as a | |
| single dose more than 3 times per week in the | |
| period 14 to 28 days prior to randomization (ie, | |
| Day −15 to Day −28) | |
| Use of hypnotics (maximum allowable dose of | Could potentially confound study results |
| 10 mg zolpidem tartrate, 1 gram chloral hydrate) | |
| at bedtime more than 3 times per week in the 28 | |
| days prior to randomization | |
| Administration of a depot antipsychotic injection | Could potentially confound study results |
| within 2 dosing intervals prior to randomization | |
| Use of potent cytochrome P450 (CYP) 3A4 | To avoid drug interaction |
| inducers (e.g., barbiturates, carbamazepine, | |
| glucocorticoids, phenytoin, rifampin, rifabutin, | |
| thioridazine, and St John's Wort) in the 14 days | |
| preceding randomization | |
| Use of potent CYP 3A4 inhibitors (e.g., | To avoid drug interaction |
| macrolide antibiotics [clarithromycin, | |
| fluvoxamine, nefazodone, erythromycin, | |
| troleandomycin]; azolantifungals [fluconazole, | |
| itraconazole, ketoconazole (except for topical | |
| use)]; protease inhibitors [indinavir, nelfinavir, | |
| ritonavir, saquinavir]) in the 14 days preceding | |
| randomization | |
| If the patient's CBC with WBC differential | To ensure safety |
| shows an ANC ≦1.5 × 10⁹/L, repeat test within | |
| 24 hours. If it remains, <1.5 × 10⁹/L, the patient | |
| will be excluded. | |
| Treatment with quetiapine for anxiety | To preserve integrity of the results |
| disorder in the 6 months prior to randomization | |
| Known history of intolerance or | To ensure safety |
| hypersensitivity to quetiapine or to any other | |
| component in the tablets | |
| Known lack of response to quetiapine, as | To preserve integrity of the results |
| judged by the investigator | To ensure safety |

Restrictions

TABLE 47
Restrictions in treatment during the study

| | Rationale |
|---|---|
| Prohibited Treatments | |
| Use of drugs that induce or inhibit the | Potentially confounds the results |
| hepatic metabolizing cytochrome 3A4 | To ensure safety |
| enzymes [e.g. inducers: carbamazepine, | |
| phenytoin, barbiturates, rifampin, rifabutin, | |
| glucocorticoids, thioridazine and St John's | |
| wort, and inhibitors: ketoconazole (except | |
| for topical use), itraconazole, fluconazole, | |
| erytromycin, clarithomycin, fluvoxamine, | |
| nefazodone, troleandomycin, indinavir, | |
| nelfinavir, ritonavir, and saquinavir]. | |
| Use of any psychoactive drugs including | Potentially confounds the results |
| antidepressant, anxiolytic, hypnotic, mood | |
| stabilizing, antipsychotic, and sedative | |
| medications other than those specifically | |
| restricted (ie, zolpidem tartrate, chloral | |
| hydrate) | |
| Prophylactic use of anticholinergics is | Potentially confounds the results |
| prohibited. | |
| Electroconvulsive therapy (ECT). | Potentially confounds the results |
| Abuse according to the DSM-IV criteria | Potentially confounds the results |
| of Alcohol, Opiates, amphetamine, | |
| barbiturate, cocaine, cannabis, or | |
| hallucinogen throughout the study | |
| Restricted Treatments | |
| Anticholinergics can be used to treat | Need to allow anticholinergics for treatment of |
| extrapyramidal symptoms. | EPS but not prophylactic as it could potentially |
| | mask EPS. |
| Psychotherapy is only allowed if it has | Potentially confounds the results |
| been ongoing since at least 3 months prior to | |
| screening | |
| From randomization until Day 14 only, | Need to allow hypnotics at reasonable doses. |
| one of the following can be used for | Differences in treatment traditions and availability |
| insomnia, maximum 2 times per week, up to | of products between countries require alternative |
| the specified dosage per night; hypnotic use | products. |
| not allowed on the night prior to conducting | |
| study assessments: | |
| zolpidem tartrate 10 mg | |
| chloral hydrate, 1 g. | |

### Example 19

### Treatment of GAD

The following study is an 8-week, Multicentre, Randomized, Double-blind, Parallel-group, Placebo-controlled, Active-controlled (Escitalopram Oxalate 10mg) Study of the Efficacy and Safety of Quetiapine Fumarate (SEROQUEL®) 150 mg/day and 300 mg/day Compared with Placebo in the treatment of GAD.

The overall rationale for this study is to demonstrate that quetiapine fumarate (SEROQUEL®) is efficacious and safe in the acute treatment of patients with GAD. This trial will serve as one of three studies to investigate the short-term efficacy and safety of quetiapine in GAD.

A primary objective of this study is to evaluate the efficacy of quetiapine fumarate (SEROQUEL®) compared to placebo in the treatment of anxiety symptoms in patients with GAD (Table 45). The secondary objectives of the study are listed in Table 49.

TABLE 48
Primary Objective

| Claims to be addressed | Primary Objective | Primary Outcome Variable |
|---|---|---|
| Quetiapine is more effective | To evaluate the efficacy of | Change from randomization in |
| than placebo in GAD as | quetiapine versus placebo in the | the Hamilton Anxiety Scale |
| measured by total HAM-A | treatment of anxiety symptoms | (HAM-A) total score at Day 57 |
| score | in patients with GAD | |

TABLE 49
Secondary Objectives

| Claims to be addressed or | | |
|---|---|---|
| hypothesis to be tested | Secondary Objective | Secondary Outcome Variables |
| Quetiapine demonstrates an | To evaluate the early efficacy of | Change from randomization in |
| anti-anxiety effect by Day 4 | quetiapine in the treatment of | the HAM-A total score at Day 4 |
| compared to placebo | anxiety symptoms in patients | Change from randomization in |
| | with GAD | HAM-A psychic cluster |
| | | (anxious mood, tension, fears, |
| | | insomnia, intellectual changes, |
| | | depressed mood, and behavior |
| | | at interview) at Day 4 |
| | | Change from randomization in |
| | | HAM-A somatic cluster |
| | | (somatic muscular, somatic |
| | | sensory, cardiovascular system, |
| | | respiratory system, |
| | | gastrointestinal system, |
| | | genitourinary system, |
| | | autonomic system) at Day 4 |
| | | Change from randomization in |
| | | Clinical Global Impression |
| | | Severity of Illness (CGI-S) |
| | | score at Day 4 |
| Quetiapine has a greater | To evaluate the early efficacy of | HAM-A Response (decrease |
| response rate at Day 4 than | quetiapine versus placebo by | from randomization total score |
| placebo | evaluating the response rate in | of = 50%) at Day 4 |
| | the treatment of anxiety | |
| | symptoms in patients with GAD | |
| Quetiapine is more effective | To evaluate the efficacy of | Change from randomization in |
| than placebo in GAD across | quetiapine versus placebo in the | (CGI-S) score at Day 57 |
| anxiety symptoms | treatment of anxiety symptoms | CGI Global Improvement (CGI- |
| | in patients with GAD | I) at Day 57 (much or very |
| | | much improved) |
| | | Change from randomization in |
| | | HAM-A psychic cluster |
| | | (anxious mood, tension, fears, |
| | | insomnia, intellectual changes, |
| | | depressed mood, and behavior |
| | | at interview) at Day 57 |
| | | Change from randomization in |
| | | HAM-A somatic cluster |
| | | (somatic muscular, somatic |
| | | sensory, cardiovascular system, |
| | | respiratory system, |
| | | gastrointestinal system, |
| | | genitourinary system, |
| | | autonomic system) at Day 57 |
| Quetiapine is at least as | To evaluate the efficacy of | Change from randomization in |
| effective as escitalopram in | quetiapine versus escitalopram | the Hamilton Anxiety Scale |
| GAD across anxiety symptoms | in the treatment of anxiety | (HAM-A) total score at Day 57 |
| | symptoms in patients with GAD | CGI Global Improvement at |
| | | Day 57 (much or very much |
| | | improved) |
| | | Change from randomization in |
| | | HAM-A psychic cluster |
| | | (anxious mood, tension, fears, |
| | | insomnia, intellectual changes, |
| | | depressed mood, and behavior |
| | | at interview) at Day 57 |
| | | Change from randomization in |
| | | HAM-A somatic cluster |
| | | (somatic muscular, somatic |
| | | sensory, cardiovascular system, |
| | | respiratory system, |
| | | gastrointestinal system, |
| | | genitourinary system, |
| | | autonomic system) at Day 57 |
| Quetiapine has a greater | To evaluate the efficacy of | HAM-A Response (decrease |
| response rate than placebo in | quetiapine versus placebo by | from randomization total score |
| patients with GAD | evaluating the response rate in | of = 50%) at Day 57 |
| | the treatment of anxiety | |
| | symptoms in patients with GAD | |
| Quetiapine is better than | To evaluate the efficacy of | HAM-A Remission (HAM-A |
| placebo at achieving remission | quetiapine versus placebo by | total score = 7) at Day 57 |
| in patients with GAD | evaluating the remission rate in | |
| | the treatment of anxiety | |
| | symptoms in patients with GAD | |
| Quetiapine is more effective | To evaluate the efficacy of | Change from randomization in |
| than placebo in reducing | quetiapine versus placebo in the | Montgomery-Asberg |
| depressive symptoms in GAD | treatment of depressive | Depression Rating Scale |
| | symptoms in patients with GAD | (MADRS) total score at Day 57 |
| Quetiapine is superior to | To evaluate the efficacy of | Change from randomization in |
| placebo in improving sleep | quetiapine versus placebo in | Pittsburgh Sleep Scale Index |
| quality in patients with GAD | improving sleep quality in | (PSQI) score at Day 57 |
| | patients with GAD | Change from randomization in |
| | | the MADRS sleep disturbance |
| | | factor (Item 4) at Day 57 |
| Quetiapine is more effective | To evaluate the effect of | Change from randomization in |
| than placebo in improving the | quetiapine versus placebo on the | Q-les-Q total score at Day 57 |
| quality of life of patients with | quality of life of patients with | Change from randomization in |
| GAD | GAD | Q-les-Q Item 16 (Overall life |
| | | satisfaction) at Day 57 |
| Quetiapine is more effective | To evaluate if quetiapine | Change from randomization in |
| than placebo in improving | improves patient satisfaction | Q-les-Q Item 15 (Satisfaction |
| patient satisfaction in patients | versus placebo in patients with | with medication) at Day 57 |
| with GAD | GAD | |
| Quetiapine once daily up to | To assess the safety and | Change from normal to |
| 300 mg/day is safe and well | tolerability of quetiapine in | Clinically Important in: |
| tolerated in patients with GAD | patients with GAD | Physical |
| Quetiapine is associated with | | Examinations |
| placebo levels of sexual | | Laboratory values |
| dysfunction | | (including glucose/lipids) |
| Quetiapine is associated with | | Vital signs |
| lower levels of sexual | | Electrocardiograms |
| dysfunction compared to | | (ECGs) |
| escitalopram | | Change from randomization in |
| Quetiapine is associated with | | the Changes in Sexual |
| placebo levels of nausea and | | Functioning Questionnaire |
| vomiting | | (CSFQ) total score at Day 57 |
| Quetiapine is associated with | | Adverse events of nausea or |
| lower levels of nausea and | | vomiting |
| vomiting compared to | | Adverse events related to EPS |
| escitalopram | | (including akathisia) |
| Quetiapine is associated with | | Change from randomization in |
| placebo levels of EPS | | the SAS and BARS scores at |
| (including akathisia) | | Day 57 |
| Quetiapine has similar | | Adverse events related to |
| incidence of withdrawals due | | discontinuation |
| to adverse events compared to | | Adverse events related to |
| escitalopram | | discontinuation |
| Quetiapine does not have | | Change in Discontinuation- |
| serious discontinuation | | Emergent Signs and Symptoms |
| symptoms | | total score at Day 64 and Day |
| Somnolence with quetiapine is | | 71 |
| generally mild, occurs early in | | Severity of AEs related to |
| treatment; is not persistent in | | somnolence |
| the majority of patients and is | | Adverse events related to |
| rarely a cause of withdrawal as | | somnolence |
| compared to placebo | | Time of first instance of |
| Quetiapine is associated with a | | somnolence |
| favorable weight profile in | | Withdrawals due to AE of |
| patients with GAD | | somnolence |
| | | Change in weight from |
| | | randomization to Day 57 |
| | | Change in waist circumference |
| | | from randomization to Day 57 |
| | | Clinically significant weight |
| | | gain (patients with ≧7% increase |
| | | from randomization weight to |
| | | Day 57) |

This is an 8-week, 4-arm, multicentre, randomized, parallel-group, double-blind, placebo-controlled, active-controlled Phase III study of the efficacy and safety of quetiapine fumarate (SEROQUEL®) 150 mg/day and 300 mg/day and escitalopram oxalate (Lexapro™) 10 mg/day compared with placebo in the treatment of GAD.

This study is comprised of three periods, the Screening Period, the Treatment Period and the Post-Treatment Period.

1) Screening Period

Eligibility for the study will be assessed at the Screening Visit. The Screening Period can extend up to 14 days prior to Day 1. Patients will undergo procedures and assessments prior to randomization.

2) Treatment Period

Eligible patients will be randomized on Day 1 (Visit 2) to one of four treatment groups: quetiapine 150 mg/day, quetiapine 300 mg/day, escitalopram 10 mg/day or placebo. Patients randomized to quetiapine will follow a dose titration scheme to reach the randomized dose level. The dose titration scheme is as follows: Day 1 and Day 2—50 mg quetiapine, Day 3 and Day 4—150 mg quetiapine and Day 5 and up—300 mg quetiapine. Patients will be dosed to their appropriate level in a blinded fashion according to their randomized treatment arm. Patients will receive 8 weeks of treatment from Day 1 to Day 56 and will undergo the procedures and assessments.

3) Post-Treatment Period

All randomized patients will be asked to call in to an Interactive Voice Response System (IVRS) for assessment of discontinuation-emergent signs and symptoms (DESS). Baseline DESS will be collected at the final study visit, Day 57. Patients will then call in to the IVRS at Day 1, Day 3, Day 5, Day 7 and Day 14 after the final study visit, Day 57. The baseline DESS assessment will be performed at the final study visit (Day 57) via IVRS at the physician's office. The rest of the assessments will be conducted at home.

Study Population

Patients will be male or female, 18 to 65 years of age, with a DSM-IV diagnosis of GAD according to DSM-IV (Diagnostic and Statistical Manual of Mental Disorders, Fourth Edition) criteria 300.02 as assessed by the MINI.

Patients are required to have a HAM-A (conducted by the Structured Interview Guide for HAM-A [SIGH-A]) total score of =20 with both Item 1 and Item 2 scores=2 at both the Screening and Randomization Visits. Patients are required to have CGI-S score=4 and MADRS score<17 at both the Screening and Randomization Visits. Additionally, patients are required to have a total COVI score=8 and greater than the total RASKIN item score at both the Screening and Randomization Visits with all individual RASKIN item scores=3 at both screening and randomization.

Number of Patients

About 800 patients will be randomized to obtain 744 evaluable patients in total, assuming that 7% of the patients will be inevaluable. Recruitment of patients will be stopped when it is determined that 744 randomized patients are evaluable. An evaluable patient will be defined as a patient who has used study medication, has a HAM-A total score at randomization and at least one HAM-A total score post-randomization.

Study Duration

Eligible patients will receive 56 days of randomized treatment. The Treatment Period will be followed by a 2-week Post-Treatment Period to measure discontinuation-emergent signs and symptoms (DESS). Patients randomized to the quetiapine treatment arms will be up titrated during the Treatment Period according to Table 47. There will be no down titration for any of the treatment arms. All study medication will stop at Day 56. There will be no study medication dispensed during the Post-Treatment Period.

Comparator

LEXAPRO® (escitalopram oxalate) (Forest Laboratories, Inc.) has obtained FDA approval for the treatment of GAD. This approval was based upon efficacy of LEXAPRO in three, 8-week, placebo-controlled trials in patients with GAD. The recommended starting dose of escitalopram oxalate for the treatment of GAD is 10 mg once a day.

Through both internal and external consultation, the clinical study team decided that the utilization of escitalopram 10mg would be acceptable as an active reference.

Drug Formulation; Doses and Dosing Regimen

The sustained release (SR) formulation of quetiapine, matching escitalopram, or matching placebo will be administered once daily, in the evening, from Day 1 with doses escalating to reach each target dose according to Table 50.

TABLE 50
Dose Escalation of Investigational Products

| | Treatment group | | | |
|---|---|---|---|---|
| | Quetiapine | Quetiapine | Escitalopram | |
| Day | 150 mg/day | 300 mg/day | 10 mg/day | Placebo |
| 1 and 2 | 50 | 50 | 10 | Placebo |
| 3 and 4 | 150 | 150 | 10 | Placebo |
| 5 and up | 150 | 300 | 10 | Placebo |

Study Procedures

Eligibility for the study will be assessed through the Screening Period and at the Randomization Visit. The patients will be randomized to treatment groups at Day 1 after fulfilling all inclusion criteria and none of the exclusion criteria.

Statistical Analysis for Primary Endpoint

Confirmatory Strategy

Primary claim: The null hypotheses are that there are no differences between quetiapine (each dose) and placebo with respect to the primary outcome, change from randomization in HAM-A total score at Day 57. There will be 2 primary comparisons tested: each quetiapine dose compared to placebo. The overall experiment type I error rate will be set to 0.05.

Key regulatory claim: Q-LES-Q total score has been identified as a key additional regulatory claim. Specifically this would include the null hypotheses that there are no differences between quetiapine (each dose) and placebo with respect to the secondary outcome, change from randomization in Q-LES-Q total score at Day 57. Again, there will be 2 key secondary comparisons tested: each quetiapine dose compared to placebo. The strategy is to control the overall experiment type I error while including these 2 additional comparisons with the 2 primary comparisons.

Multiple Comparisons Procedure

In order to take account of these 4 comparisons, a parallel gatekeeper approach will be used. The primary hypotheses serve as a gatekeeper in the sense that the key secondary hypotheses of interest (Q-LES-Q) will be tested only after the primary analysis has yielded a statistically significant result. Using a gatekeeping strategy for testing the primary and secondary hypotheses will preserve the overall experiment type I error rate at 0.05. Weights will be applied equally within the primary claim hypotheses (i.e. HAM-A comparisons) and set at 0.5. Similarly, weights will be applied equally within the key secondary claim hypotheses (i.e. Q-LES-Q comparisons) and set at 0.5.

The comparison between active control (escitalopram oxalate) and placebo will not be part of the confirmatory strategy. This comparison will be used to assess internal evidence of assay sensitivity.

Analysis Populations

The efficacy analyses will be based on the modified intention-to-treat population (Full Analysis Set). This population will include all randomized subjects, classified according to randomized treatment, who took study medication and who have a randomization HAM-A total score assessments and at least one HAM-A total score post-randomization.

The safety displays will be based on the safety population. This population includes all randomized subjects who took study medication, classified according to the treatment actually received.

Analysis of the Primary Outcome Variable

The primary efficacy outcome variable will be analyzed using an analysis of covariance (ANCOVA) model including the randomization HAM-A total score, centre, and treatment group as variables in the analysis.

Missing Data

Last observation carried forward (LOCF) is defined in the following way: the latest post randomization valid value before the missing data will be used. Randomization values will not be carried forward. LOCF methodology will be used for the primary outcome variable.

Rationale for Sample Size

The study is powered to show that either dose of quetiapine 150 mg or 300 mg is different from placebo with respect to the primary efficacy outcome variable, change from randomization in HAM-A total score at Day 57 (Table 51).

TABLE 51
Sample size calculation

| | |
|---|---|
| Power | 90% |
| Difference to be detected | 2.75 |
| Standard deviation | 7.5 |
| Significance level | 5% Overall: Bonferroni each |
| | comparison at 0.025 |
| Total sample size (each group) | 744 (186) |

An evaluable subject will be defined as a subject who has used study medication, has a HAM-A total score at randomization and at least one HAM-A total score post randomization).

Note: No adjustment for the comparison of active control vs. placebo has been made as this is not one of the primary hypotheses.

The sample size estimate was based on other 8-week GAD trials. Maximum treatment differences observed in various Effexor XR trials (NDA 20-699/S-001) ranged from 2.3 to 2.9 points. Similar results were noted in a published paroxetine trial. Standard deviations and non-evaluable rates from these trials ranged from 7.2 to 8.8 points and 1% to 12% respectively.

Study-Specific Inclusion/Exclusion Criteria, Restrictions and Rationale

TABLE 52
Inclusion Criteria: including but not limited to the following:

| Inclusion Criteria | Rationale |
|---|---|
| Male or female aged 18 to 65 years | To include adult |
| | subjects |
| A documented clinical diagnosis of GAD | Selection of patients |
| according to DSM-IV (Diagnostic and | whose anxiety is not |
| Statistical Manual of Mental Disorders, | part of another disorder |
| Fourth Edition) criteria 300.02 as | |
| assessed by the MINI (Mini-International | |
| Neuropsychiatric Interview) | |
| A HAMA total score ≧20 with Item 1 (anxious | To ensure that patients |
| mood) and Item 2 (tension) scores ≧2 at both | are sufficiently acutely ill |
| screening and randomization | |
| A CGI-S score ≧4 at both screening and | To ensure that patients |
| randomization | are sufficiently acutely ill |
| A COVI total score ≧8 and greater than the | To avoid selection of |
| RASKIN total score at both screening and at | patients with depression |
| randomization and all RASKIN item scores = 3 | |
| at screening and randomization | |

TABLE 53
Exclusion Criteria

| Exclusion Criteria | Rationale |
|---|---|
| Patients with a current DSM-IV Axis I disorder | To exclude other diagnoses which may confound |
| other than GAD (with or without simple phobia) | results |
| within 6 months of screening | |
| The presence or history of any psychotic disorder | Not part of the target population, would potentially |
| | confound the results |
| The presence of any DSM-IV axis II disorder that | Not part of the target population |
| is likely to interfere with the patient's ability to | |
| participate in the study as judged by the | |
| investigator | |
| Patients suffering from depressive symptoms, | Not part of the target population |
| defined as having a MADRS total score ≧17 at | |
| either screening or randomization | |
| Patients who, in the investigator's judgment, pose | To ensure safety |
| a current serious suicidal or homicidal risk or have | |
| made a suicide attempt | |
| Evidence of clinically relevant disease (e.g., renal | Could potentially confound study results |
| or hepatic impairment, significant coronary artery | To ensure safety |
| disease, cerebrovascular disease, viral hepatitis B | |
| or C, acquired immunodeficiency syndrome | |
| [AIDS], or cancer), or a clinical finding that is | |
| unstable or, in the opinion of the investigator, | |
| would either be negatively affected by the study | |
| medication or would affect the study medication | |
| History of seizure disorder, except febrile | To ensure safety |
| convulsions | |
| Substance or alcohol abuse or dependence, as | To ensure protocol compliance and generate |
| defined in DSM-IV criteria, within 6 months prior | evaluable data |
| to screening | |
| Use of antipsychotic medication within 28 days | Could potentially confound study results |
| prior to randomization. | |
| Use of benzodiazepines, antidepressants, MAO | Could potentially confound study results |
| inhibitors and mood stabilizers within 14 days | |
| prior to randomization. | |
| Use of benzodiazepines (maximum allowable | Could potentially confound study results |
| dose of 10-mg equivalent of diazepam) as a single | |
| dose more than 3 times per week in the period 14 | |
| to 28 days prior to randomization (ie, Day −15 to | |
| Day −28) | |
| Use of hypnotics (maximum allowable dose of 10 mg | Could potentially confound study results |
| zolpidem tartrate, 1 gram chloral hydrate) at | |
| bedtime more than 3 times per week in the 28 | |
| days prior to randomization | |
| Administration of a depot antipsychotic injection | Could potentially confound study results |
| within 2 dosing intervals prior to randomization | |
| Use of potent cytochrome P450 (CYP) 3A4 | To avoid drug interaction |
| inducers (e.g., barbiturates, carbamazepine, | |
| glucocorticoids, phenytoin, rifampin, rifabutin, | |
| thioridazine and St John's Wort) in the 14 days | |
| preceding randomization | |
| Use of potent CYP 3A4 inhibitors (e.g., macrolide | To avoid drug interaction |
| antibiotics [clarithromycin, fluvoxamine, | |
| nefazodone, erythromycin, troleandomycin]; | |
| azolantifungals [fluconazole, itraconazole, | |
| ketoconazole (except for topical use)]; protease | |
| inhibitors [indinavir, nelfinavir, ritonavir, | |
| saquinavir]) in the 14 days preceding | |
| randomization | |
| Treatment with quetiapine for anxiety disorder in | To preserve integrity of the results |
| the 6 months prior to randomization | |

### Example 20

### Treatment of GAD

The following study is an 8-week, multicentre, randomized, double-blind, parallel-group, placebo-controlled, active-controlled (Paroxetine 20 mg) study of the efficacy and safety of quetiapine fumarate (SEROQUEL®) 50 mg/day and 150 mg/day compared with placebo in the treatment of GAD.

The overall rationale for this study is to demonstrate that quetiapine fumarate (SEROQUEL®) is efficacious and safe in the acute treatment of patients with GAD. This trial will serve as one of three studies to investigate the short-term efficacy and safety of quetiapine in GAD.

The primary objective of this study is to evaluate the efficacy of quetiapine fumarate (SEROQUEL®) compared to placebo in the treatment of anxiety symptoms in patients with GAD (Table 5 1). The secondary objectives of the study are listed in Table 52.

TABLE 54
Primary Objective

| Claims to be addressed | Primary Objective | Primary Outcome Variable |
|---|---|---|
| Quetiapine is more effective | To evaluate the efficacy of | Change from randomization in |
| than placebo in GAD as | quetiapine versus placebo in the | the Hamilton Anxiety Scale |
| measured by total HAM-A | treatment of anxiety symptoms | (HAM-A) total score at Day 57 |
| score | in patients with GAD | |

TABLE 55
Secondary Objectives

| Claims to be addressed or | | |
|---|---|---|
| hypothesis to be tested | Secondary Objective | Secondary Outcome Variables |
| Quetiapine demonstrates an | To evaluate the early efficacy of | Change from randomization in |
| anti-anxiety effect by Day 4 | quetiapine in the treatment of | the HAM-A total score at Day 4 |
| compared to placebo | anxiety symptoms in patients | Change from randomization in |
| | with GAD | HAM-A psychic cluster |
| | | (anxious mood, tension, fears, |
| | | insomnia, intellectual changes, |
| | | depressed mood, and behavior |
| | | at interview) at Day 4 |
| | | Change from randomization in |
| | | HAM-A somatic cluster |
| | | (somatic muscular, somatic |
| | | sensory, cardiovascular system, |
| | | respiratory system, |
| | | gastrointestinal system, |
| | | genitourinary system, |
| | | autonomic system) at Day 4 |
| | | Change from randomization in |
| | | Clinical Global Impression |
| | | Severity of Illness (CGI-S) |
| | | score at Day 4 |
| Quetiapine has a greater | To evaluate the early efficacy of | HAM-A Response (decrease |
| response rate at Day 4 than | quetiapine versus placebo by | from randomization total score |
| placebo | evaluating the response rate in | of ≧50%) at Day 4 |
| | the treatment of anxiety | |
| | symptoms in patients with GAD | |
| Quetiapine is more effective | To evaluate the efficacy of | Change from randomization in |
| than placebo in GAD across | quetiapine versus placebo in the | CGI-S score at Day 57 |
| anxiety symptoms | treatment of anxiety symptoms | CGI Global Improvement (CGI- |
| | in patients with GAD | I) at Day 57 (much or very |
| | | much improved) |
| | | Change from randomization in |
| | | HAM-A psychic cluster |
| | | (anxious mood, tension, fears, |
| | | insomnia, intellectual changes, |
| | | depressed mood, and behavior |
| | | at interview) at Day 57 |
| | | Change from randomization in |
| | | HAM-A somatic cluster |
| | | (somatic muscular, somatic |
| | | sensory, cardiovascular system, |
| | | respiratory system, |
| | | gastrointestinal system, |
| | | genitourinary system, |
| | | autonomic system) at Day 57 |
| Quetiapine is at least as | To evaluate the efficacy of | Change from randomization in |
| effective as paroxetine in GAD | quetiapine versus paroxetine in | the Hamilton Anxiety Scale |
| across anxiety symptoms | the treatment of anxiety | (HAM-A) total score at Day 57 |
| | symptoms in patients with GAD | CGI Global Improvement at |
| | | Day 57 (much or very much |
| | | improved) |
| | | Change from randomization in |
| | | HAM-A psychic cluster |
| | | (anxious mood, tension, fears, |
| | | insomnia, intellectual changes, |
| | | depressed mood, and behavior |
| | | at interview) at Day 57 |
| | | Change from randomization in |
| | | HAM-A somatic cluster |
| | | (somatic muscular, somatic |
| | | sensory, cardiovascular system, |
| | | respiratory system, |
| | | gastrointestinal system, |
| | | genitourinary system, |
| | | autonomic system) at Day 57 |
| Quetiapine has a greater | To evaluate the efficacy of | HAM-A Response (decrease |
| response rate than placebo in | quetiapine versus placebo by | from randomization total score |
| patients with GAD | evaluating the response rate in | of ≧50%) at Day 57 |
| | the treatment of anxiety | |
| | symptoms in patients with GAD | |
| Quetiapine is better than | To evaluate the efficacy of | HAM-A Remission (HAM-A |
| placebo at achieving remission | quetiapine versus placebo by | total score ≦7) at Day 57 |
| in patients with GAD | evaluating the remission rate in | |
| | the treatment of anxiety | |
| | symptoms in patients with GAD | |
| Quetiapine is more effective | To evaluate the efficacy of | Change from randomization in |
| than placebo in reducing | quetiapine versus placebo in the | Montgomery-Asberg |
| depressive symptoms in GAD | treatment of depressive | Depression Rating Scale |
| | symptoms in patients with GAD | (MADRS) total score at Day 57 |
| Quetiapine is superior to | To evaluate the efficacy of | Change from randomization in |
| placebo in improving sleep | quetiapine versus placebo in | Pittsburgh Sleep Scale Index |
| quality in patients with GAD | improving sleep quality in | (PSQI) score at Day 57 |
| | patients with GAD | Change from randomization in |
| | | the MADRS sleep disturbance |
| | | factor (Item 4) at Day 57 |
| Quetiapine is more effective | To evaluate the effect of | Change from randomization in |
| than placebo in improving the | quetiapine versus placebo on the | Q-les-Q total score at Day 57 |
| quality of life of patients with | quality of life of patients with | Change from randomization in |
| GAD | GAD | Q-les-Q Item 16 (Overall life |
| | | satisfaction) at Day 57 |
| Quetiapine is more effective | To evaluate if quetiapine | Change from randomization in |
| than placebo in improving | improves patient satisfaction | Q-les-Q Item 15 (Satisfaction |
| patient satisfaction in patients | versus placebo in patients with | with medication) at Day 57 |
| with GAD | GAD | |
| Quetiapine once daily up to | To assess the safety and | Change from normal to |
| 150 mg/day is safe and well | tolerability of quetiapine in | Clinically Important in: Physical |
| tolerated in patients with GAD | patients with GAD | Examinations; Laboratory |
| Quetiapine is associated with | | values (including |
| placebo levels of sexual | | glucose/lipids); Vital signs; |
| dysfunction | | Electrocardiograms (ECGs) |
| Quetiapine is associated with | | Change from randomization |
| lower levels of sexual | | in the Changes in Sexual |
| dysfunction compared to | | Functioning Questionnaire |
| paroxetine | | (CSFQ) total score at Day 57 |
| Quetiapine is associated with | | Adverse events of nausea or |
| placebo levels of nausea and | | vomiting |
| vomiting | | Adverse events related to |
| Quetiapine is associated with | | EPS (including akathisia) |
| lower levels of nausea and | | Change from randomization |
| vomiting compared to | | in the SAS and BARS scores |
| paroxetine | | at Day 57 |
| Quetiapine is associated with | | Adverse events related to |
| placebo levels of EPS | | discontinuation |
| (including akathisia) | | Change in Discontinuation- |
| Quetiapine has similar | | Emergent Signs and |
| incidence of withdrawals due | | Symptoms total score at Day |
| to adverse events compared to | | 64 and Day 71 |
| paroxetine | | Severity of AEs related to |
| Quetiapine does not have | | somnolence |
| serious discontinuation | | Adverse events related to |
| symptoms | | somnolence |
| Somnolence with quetiapine is | | Time of first instance of |
| generally mild, occurs early in | | somnolence |
| treatment; is not persistent in | | Withdrawals due to AE of |
| the majority of patients and is | | somnolence |
| rarely a cause of withdrawal as | | Change in weight from |
| compared to placebo | | randomization to Day 57 |
| Quetiapine is associated with a | | Change in waist |
| favorable weight profile in | | circumference from |
| patients with GAD | | randomization to Day 57 |
| | | Clinically significant weight |
| | | gain (patients with ≧7% |
| | | increase from randomization |
| | | weight to Day 57) |

This is an 8-week, 4-arm, multicentre, randomized, parallel-group, double-blind, placebo-controlled, active-controlled Phase III study of the efficacy and safety of quetiapine fumarate (SEROQUEL®) 50 mg/day and 150 mg/day and paroxetine hydrochloride (Paxil®) 20 mg/day compared with placebo in the treatment of GAD.

This study is comprised of three periods, the Screening Period, the Treatment Period and the Post-Treatment Period.

1) Screening Period

Eligibility for the study will be assessed at the Screening Visit. The Screening Period can extend up to 14 days prior to Day 1.

2) Treatment Period

Eligible patients will be randomized on Day 1 (Visit 2) to one of four treatment groups: quetiapine 50 mg/day, quetiapine 150 mg/day, paroxetine 20 mg/day or placebo. Patients randomized to quetiapine will follow a dose titration scheme to reach the randomized dose level. The dose titration scheme is as follows: Day 1 and Day 2—50 mg quetiapine, Day 3 and up—150 mg quetiapine. Patients will be dosed to their appropriate level in a blinded fashion according to their randomized treatment arm. Patients will receive 8 weeks of treatment from Day 1 to Day 56.

3) Post-Treatment Period

All randomized patients will be assessed for discontinuation-emergent signs and symptoms (DESS). Baseline DESS will be collected at the final study visit, Day 57. The baseline DESS assessment will be performed at the final study visit (Day 57).

Study Population

Patients will be male or female, 18 to 65 years of age, with a DSM-IV diagnosis of GAD according to DSM-IV (Diagnostic and Statistical Manual of Mental Disorders, Fourth Edition) criteria 300.02 as assessed by the MINI.

Patients are required to have a HAM-A (conducted by the Structured Interview Guide for HAM-A [SIGH-A]) total score of ≧20 with both Item 1 and Item 2 scores≧2 at both the Screening and Randomization Visits. Patients are required to have CGI-S score≧4 and MADRS score<17 at both the Screening and Randomization Visits. Additionally, patients are required to have a total COVI score≧8 and greater than the total RASKIN item score at both the Screening and Randomization Visits with all individual RASKIN item scores≦3 at both screening and randomization.

Study Duration

Eligible patients will receive 56 days of randomized treatment. The Treatment Period will be followed by a 2-week Post-Treatment Period to measure discontinuation-emergent signs and symptoms (DESS). Patients randomized to the quetiapine treatment arms will be up titrated during the Treatment Period according to Table below. There will be no down titration for any of the treatment arms. All study medication will stop at Day 56. There will be no study medication dispensed during the Post-Treatment Period.

Comparator

PAXIL® (paroxetine hydrochloride) (Glaxo SmithKline) has obtained FDA approval for the treatment of GAD. This approval was based upon efficacy of PAXIL® in two, 8-week, placebo-controlled trials in patients with GAD. The recommended starting dose of paroxetine hydrochloride for the treatment of GAD is 20 mg once a day.

Through both internal and external consultation, the clinical study team decided that the utilization of paroxetine 20 mg would be acceptable as an active reference.

Drug Formulation; Doses and Dosing Regimen

The sustained release (SR) formulation of quetiapine, matching paroxetine, or matching placebo will be administered once daily, in the evening, from Day 1 with doses escalating to reach each target dose according to the Table below.

Tablets to be used in the study are: 50 mg quetiapine sustained release (SR) tablets; 20 mg paroxetine tablets; placebo tablets to match.

TABLE 56
Dose Escalation of Investigational Products

Treatment group

| | Quetiapine | Quetiapine | Paroxetine | |
|---|---|---|---|---|
| Day | 50 mg/day | 150 mg/day | 20 mg/day | Placebo |
| 1 and 2 | 50 | 50 | 20 | Placebo |
| 3 and up | 50 | 150 | 20 | Placebo |

Study Procedures

Eligibility for the study will be assessed through the Screening Period and at the Randomization Visit. The patients will be randomized to treatment groups at Day 1 after fulfilling all inclusion criteria and none of the exclusion criteria.

Statistical Analysis for Primary Endpoint

Confirmatory Strategy

Primary claim: The null hypotheses are that there are no differences between quetiapine (each dose) and placebo with respect to the primary outcome, change from randomization in HAM-A total score at Day 57. There will be 2 primary comparisons tested: each quetiapine dose compared to placebo. The overall experiment type I error rate will be set to 0.05.

Key Regulatory Claim:

Q-LES-Q total score has been identified as a key additional regulatory claim. Specifically this would include the null hypotheses that there are no differences between quetiapine (each dose) and placebo with respect to the secondary outcome, change from randomization in Q-LES-Q total score at Day 57. Again, there will be 2 key secondary comparisons tested: each quetiapine dose compared to placebo. The strategy is to control the overall experiment type I error while including these 2 additional comparisons with the 2 primary comparisons.

Multiple Comparisons Procedure:

In order to take account of these 4 comparisons, a parallel gatekeeper approach will be used. The primary hypotheses serve as a gatekeeper in the sense that the key secondary hypotheses of interest (Q-LES-Q) will be tested only after the primary analysis has yielded a statistically significant result. Using a gatekeeping strategy for testing the primary and secondary hypotheses will preserve the overall experiment type I error rate at 0.05. Weights will be applied equally within the primary claim hypotheses (i.e. HAM-A comparisons) and set at 0.5. Similarly, weights will be applied equally within the key secondary claim hypotheses (i.e. Q-LES-Q comparisons) and set at 0.5.

The comparison between active control (paroxetine hydrochloride) and placebo will not be part of the confirmatory strategy. This comparison will be used to assess internal evidence of assay sensitivity.

Analysis Populations

The efficacy analyses will be based on the modified intention-to-treat population (Full Analysis Set). This population will include all randomized subjects, classified according to randomized treatment, who took study medication and who have a randomization HAM-A total score assessments and at least one HAM-A total score post-randomization.

The safety displays will be based on the safety population. This population includes all randomized subjects who took study medication, classified according to the treatment actually received.

Analysis of the Primary Outcome Variable

The primary efficacy outcome variable will be analyzed using an analysis of covariance (ANCOVA) model including the randomization HAM-A total score, centre, and treatment group as variables in the analysis.

Missing Data:

Last observation carried forward (LOCF) is defined in the following way: the latest post randomization valid value before the missing data will be used. Randomization values will not be carried forward.

LOCF methodology will be used for the primary outcome variable.

Rationale for Sample Size

The study is powered to show that either dose of quetiapine 50 mg or 150 mg is different from placebo with respect to the primary efficacy outcome variable, change from randomization in HAM-A total score at Day 57 (5).

TABLE 57
Sample size calculation

| | |
|---|---|
| Power | 90% |
| Difference to be detected | 2.75 |
| Standard deviation | 7.5 |
| Significance level | 5% Overall: Bonferroni each |
| | comparison at .025 |
| Total sample size (each group) | 744 (186) |

An evaluable subject will be defined as a subject who has used study medication, has a HAM-A total score at randomization and at least one HAM-A total score post randomization).

The sample size estimate was based on other 8-week GAD trials. Maximum treatment differences observed in various Effexor XR trials (NDA 20-699/S-001) ranged from 2.3 to 2.9 points. Similar results were noted in a published paroxetine trial. Standard deviations and non-evaluable rates from these trials ranged from 7.2 to 8.8 points and 1% to 12% respectively.

Study-Specific Inclusion/Exclusion Criteria, Restrictions and Rationale

TABLE 58
Inclusion Criteria

| Inclusion Criteria | Rationale |
|---|---|
| Male or female aged 18 to 65 years | To include adult subjects |
| A documented clinical diagnosis of GAD | Selection of patients |
| according to DSM-IV (Diagnostic and | whose anxiety is not |
| Statistical Manual of Mental Disorders, | part of another disorder |
| Fourth Edition) criteria 300.02 as | |
| assessed by the MINI (Mini-International | |
| Neuropsychiatric Interview) | |
| A HAMA total score ≧20 with Item 1 (anxious | To ensure that patients |
| mood) and Item 2 (tension) scores ≧2 at both | are sufficiently acutely ill |
| screening and randomization | |
| A CGI-S score ≧4 at both screening and | To ensure that patients |
| randomization | are sufficiently acutely ill |
| A COVI total score ≧8 and greater than the | To avoid selection of |
| RASKIN total score at both screening and at | patients with depression |
| randomization and all RASKIN item scores ≦3 | |
| at screening and randomization | |

TABLE 59
Exclusion Criteria

| Exclusion Criteria | Rationale |
|---|---|
| Patients with a current DSM-IV Axis I disorder | To exclude other diagnoses which may confound |
| other than GAD (with or without simple phobia) | results |
| within 6 months of screening | |
| Substance or alcohol abuse or dependence, as | To ensure protocol compliance and generate |
| defined in DSM-IV criteria, within 6 months | evaluable data |
| prior to screening (except dependence in full | |
| remission, caffeine dependence, or nicotine | |
| dependence). | |
| Use of antipsychotic medication within 28 days | Could potentially confound study results |
| prior to randomization. | |
| Use of benzodiazepines, antidepressants, MAO | Could potentially confound study results |
| inhibitors and mood stabilizers within 14 days | |
| prior to randomization. | |
| Use of benzodiazepines (maximum allowable | Could potentially confound study results |
| dose of 10-mg equivalent of diazepam) as a | |
| single dose more than 3 times per week in the | |
| period 14 to 28 days prior to randomization (ie, | |
| Day −15 to Day −28) | |
| Administration of a depot antipsychotic injection | Could potentially confound study results |
| within 2 dosing intervals prior to randomization | |
| Use of potent cytochrome P450 (CYP) 3A4 | To avoid drug interaction |
| inducers (e.g., barbiturates, carbamazepine, | |
| glucocorticoids, phenytoin, rifampin, rifabutin, | |
| thioridazine, and St John's Wort) in the 14 days | |
| preceding randomization | |
| Use of potent CYP 3A4 inhibitors (e.g., | To avoid drug interaction |
| macrolide antibiotics [clarithromycin, | |
| fluvoxamine, nefazodone, erythromycin, | |
| troleandomycin]; azolantifungals [fluconazole, | |
| itraconazole, ketoconazole (except for topical | |
| use)]; protease inhibitors [indinavir, nelfinavir, | |
| ritonavir, saquinavir]) in the 14 days preceding | |
| randomization | |

Restrictions

TABLE 60
Restrictions in treatments during the study

| Prohibited Treatments | Rationale |
|---|---|
| Use of drugs that induce or inhibit the | Potentially confounds the |
| hepatic metabolizing cytochrome 3A4 | results |
| enzymes [e.g. inducers: carbamazepine, | To ensure safety |
| phenytoin, barbiturates, rifampin, rifabutin, | |
| glucocorticoids, thioridazine and St John's | |
| wort, and inhibitors: ketoconazole (except | |
| for topical use), itraconazole, fluconazole, | |
| erytromycin, clarithomycin, fluvoxamine, | |
| nefazodone, troleandomycin, indinavir, | |
| nelfinavir, ritonavir, and saquinavir]. | |
| Use of any psychoactive drugs including | Potentially confounds the |
| antidepressant, anxiolytic, hypnotic, mood | results |
| stabilizing, antipsychotic, and sedative | |
| medications other than those specifically | |
| restricted (ie, zolpidem tartrate, chloral | |
| hydrate) | |
| Abuse according to the DSM-IV criteria | Potentially confounds the |
| of Alcohol, Opiates, amphetamine, | results |
| barbiturate, cocaine, cannabis, or | |
| hallucinogen throughout the study | |

### Example 21

### Treatment of Patients with MDD

This study is a 6-week multicentre, double-blind, randomized, parallel-group, placebo-controlled Phase III Study of the efficacy and safety of quetiapine fumarate sustained release 150 mg/day and 300 mg/day in combination with an anti-depressant in the treatment of patients with MDD with inadequate response to an antidepressant treatment.

The objective for this study is to evaluate that a sustained release form of quetiapine in combination with an anti-depressant is efficacious and safe in the treatment of MDD in patients who have inadequate response to an antidepressant. The primary objective is described in Table 61 and the secondary objectives of the study are shown in Table 62.

TABLE 61
Primary objective, corresponding outcome variables and claims

| Claims to be addressed | Primary Objective | Outcome Variable |
|---|---|---|
| 2.2 A sustained release form | To evaluate the efficacy of | Primary variable: |
| of quetiapine once daily in | sustained release form of | Change from randomization to Week 6 in |
| combination with an anti- | quetiapine in combination with | the Montgomery-Åsberg Depression |
| depressant has superior | an anti-depressant versus an | Rating Scale (MADRS) total score |
| efficacy to an anti-depressant | anti-depressant alone in patients | Secondary variables supporting the |
| alone in depression | with MDD. | primary objective: |
| 2.4 A sustained release form | | Change from randomization to each |
| of quetiapine once daily in | | assessment in the MADRS total score |
| combination with an anti- | | MADRS response, defined as a ≧50% |
| depressant has a greater | | reduction from randomization in the |
| response rate than an anti- | | MADRS total score at Week 6. |
| depressant alone in depression | | MADRS remission, defined as total score |
| 2.6 A sustained release form | | ≦8 at Week 6. |
| of quetiapine once daily in | | Change from randomization week 6 in the |
| combination with an anti- | | Hamilton Depression scale (HAM-D) total |
| depressant is better than an | | score and the HAM-D Item 1. |
| anti-depressant alone in | | Change from randomization to each |
| achieving remission in patients | | assessment in the Clinical Global |
| with depression | | Impression - Severity (CGI-S) |
| 9.1 Starting on day 1 A | | Clinical Global Impression - Improvement |
| sustained release form of | | (CGI-I) at each assessment |
| quetiapine once daily in | | |
| combination with an anti- | | |
| depressant can be prescribed at | | |
| a therapeutically effective | | |
| dose in depression | | |

TABLE 62
Secondary objectives, corresponding outcome variables and claims

| Claims to be addressed or | | |
|---|---|---|
| hypothesis to be tested | Secondary Objective | Outcome Variables |
| Efficacy | | |
| 3.2 A sustained release form | To evaluate if sustained release | Change from randomization to each |
| of quetiapine once daily in | form of quetiapine in | assessment in Hamilton Rating scale for |
| combination with an anti- | combination with an anti- | Anxiety (HAM-A) |
| depressant is more effective | depressant reduces anxiety | Change in HAM-A psychic anxiety factors |
| than an anti-depressant alone | symptoms in patients with | (Anxious Mood, Tension, Fears, Insomnia, |
| in reducing anxiety symptoms | MDD, compared to an anti- | Intellect, Depressed Mood, Behaviour at |
| in depression | depressant alone. | interview) from randomization to each |
| | | assessment |
| | | Change in HAM-D anxiety factors (item |
| | | 10 and 11) from randomization to Week 6. |
| 4.2 A sustained release form | To evaluate if sustained release | Change in HAM-D sleep disturbance |
| of quetiapine once daily in | form of quetiapine in | factors (Items 4-6) from randomization to |
| combination with an anti- | combination with an anti- | Week 6 |
| depressant is more effective | depressant improves sleep | Change in Pittsburgh Sleep Quality Index |
| than an anti-depressant alone | quality in patients with MDD, | (PSQI) global score from randomization to |
| in improving sleep onset and | compared to an anti-depressant | each assessment |
| sleep maintenance in | alone. | |
| depression | | |
| 5.2 A sustained release form | To evaluate if sustained release | Change from randomization to each |
| of quetiapine once daily in | form of quetiapine in | assessment in MADRS item 10, suicidal |
| combination with an anti- | combination with an anti- | thought |
| depressant is more effective | depressant is effective in | |
| than an anti-depressant alone | reducing suicidal ideation in | |
| in reducing suicide ideation in | patients with MDD, compared | |
| patients with depression | to an anti-depressant alone. | |
| 8.2 A sustained release form | To evaluate if sustained release | Change in HAM-A somatic anxiety factors |
| of quetiapine once daily in | form of quetiapine in | (Somatic Muscular, Somatic Sensory, |
| combination with an anti- | combination with an anti- | Cardiovascular, Respiratory, |
| depressant is more effective | depressant improves somatic | Gastrointestinal, Genitourinary, |
| than an anti-depressant alone | symptoms in the treatment of | Autonomic) from randomization to each |
| in improving somatic | patients with MDD, compared | assessment. |
| symptoms such as back pain, | to an anti-depressant alone.. | |
| headache, muscle pain, | | |
| unspecified pain, abdominal | | |
| pain, chest pain, in patients | | |
| with depression | | |
| Efficacy-Quality of Life | | |
| 6.2 A sustained release form | To evaluate if sustained release | Change from baseline to each assessment |
| of quetiapine once daily in | form of quetiapine in | in Q-les-Q total score (item 1-14). |
| combination with an anti- | combination with an anti- | Change from baseline to each assessment |
| depressant is more effective | depressant improves the quality | in Q-les-Q Item 16 (Overall quality of |
| than an anti-depressant alone | of life of patients with MDD, | life). |
| in improving the quality of life | compared to an anti-depressant | |
| of patients with depression | alone. | |
| 7.2 A sustained release form | To evaluate if sustained release | Change from randomization to each |
| of quetiapine once daily in | form of quetiapine in | assessment in Q-les-Q Item 15 |
| combination with an anti- | combination with an anti- | (Satisfaction with medication) |
| depressant is more effective | depressant improves patient | |
| than an anti-depressant alone | satisfaction in patients with | |
| in improving patient | MDD, compared to an anti- | |
| satisfaction in patients with | depressant alone. | |
| depression | | |
| Safety/tolerability | | |
| 11.1 A sustained release form | To evaluate if sustained release | Change from normal to Clinically |
| of quetiapine once daily in | form of quetiapine in | Important in: |
| combination with an anti- | combination with an anti- | Physical Examinations |
| depressant up to 300 mg/d is | depressant is safe and well- | Laboratory values (including |
| well tolerated in patients with | tolerated in the treatment of | glucose/lipids) |
| depression | patients with MDD. | Vital signs |
| 11.10 Fasting Glucose and | | Electrocardiograms (ECGs) |
| Lipids not significantly | | Adverse Events |
| elevated | | AEs leading to withdrawal |
| 11.8 A sustained release form | | Serious discontinuation symptoms |
| of quetiapine once daily in | | assessed by DESS (discontinuation scale) |
| combination with an anti- | | Incidence of AEs related to somnolence |
| depressant does not have | | Severity of somnolence reports |
| serious discontinuation | | Time of AE somnolence reports |
| symptoms | | Withdrawals due to AE of somnolence |
| 11.9 Somnolence with A | | |
| sustained release form of | | |
| quetiapine once daily in | | |
| combination with an anti- | | |
| depressant is generally mild, | | |
| occurs early in treatment; is | | |
| not persistent in the majority | | |
| of patients and is rarely a | | |
| cause of withdrawal | | |
| 11.13 A sustained release form | To evaluate if sustained release | Change in weight from randomization to |
| of quetiapine once daily in | form of quetiapine in | each assessment |
| combination with an anti- | combination with an anti- | Change in waist circumference from |
| depressant is associated with a | depressant is safe and well- | randomization to each assessment |
| favourable weight profile | tolerated in the treatment of | Proportion of patients with a ≧7% increase |
| | patients with MDD. | from randomization weight. |
| 11.2 A sustained release form | For 11.2, 11.4, 11.6, and 11.12 | AEs (especially related to sexual |
| of quetiapine once daily in | To evaluate if sustained release | dysfunction, nausea, vomiting, EPS |
| combination with an anti- | form of quetiapine in | including akathisia) |
| depressant is associated with | combination with an anti- | Change from randomization to each |
| levels of sexual dysfunction as | depressant is as safe and well- | assessment in Sexual Functioning |
| an anti-depressant alone | tolerated as an anti-depressant | Questionnaire (CSFQ) total score |
| 11.4 A sustained release form | alone in the treatment of | Change in SAS and BARS from |
| of quetiapine once daily in | patients with MDD | randomization to each assessment |
| combination with an anti- | | MADRS item 10 score ≧4 at any time after |
| depressant is associated with | | randomization or AE of suicidality/suicidal |
| levels of nausea and vomiting | | ideation/suicide attempts/suicide |
| as an anti-depressant alone | | completion |
| 11.6 A sustained release form | | Analysis of suicidality according to FDA |
| of quetiapine once daily in | | guidance <<tbc>> |
| combination with an anti- | | |
| depressant is associated with | | |
| levels of EPS (including | | |
| akathisia) as an anti- | | |
| depressant alone | | |
| 11.12 A sustained release form | | |
| of quetiapine once daily in | | |
| combination with an anti- | | |
| depressant is associated with a | | |
| lower incidence of treatment | | |
| emergent suicidal ideation | | |
| compared with an anti- | | |
| depressant alone | | |
| Pharmacokinetics | | |
| — | To evaluate if sustained release | Change in concentration of antidepressant |
| | form of quetiapine in | from randomization to Week 4 |
| | combination with an | |
| | antidepressant changes the level | |
| | of antidepressant in the | |
| | circulation | |

This is a 6-week Randomized, Parallel-group, Placebo-controlled Study of the Efficacy and Safety of quetiapine fumarate (SEROQUEL®) in combination with an anti-depressant in the Treatment of Patients with MDD with inadequate response to an antidepressant treatment.

The study comprises the following three periods:

1) Washout Period

Patients shall be evaluated and meet the DSM-IV diagnosis confirmed by the MINI and will undergo enrolment assessments at Visit 1, eligible patients will commence a washout during which prohibited medication should be washed out prior to randomization.

Patients should be on stable treatment with an adequate anti-depressant treatment (sertraline, paroxetine, venlafaxine, citralopram, escitralopram, fluoxetine, bupropion, amitryptyline or duloxetine) at least min dose according to label for 6 weeks including at least one dose increase when permitted according to label as well as having a HAMD score of at least 20 to be eligible. For verification of HAMD scores a system to systematically review the scores will be set up, as to prevent scale inflation.

2) Six-Week Double-Blind, Randomized, Placebo Controlled Treatment Period (Day 1 to Day 43)

Eligible patients will be randomized on Day 1 (Visit 2) to one of three treatment groups: sustained release form of quetiapine 150 mg/day, sustained release form of quetiapine 300 mg/day or placebo as add-on therapy to ongoing anti-depressant treatment. The ongoing treatment with the anti-depressant should be stable when entering the study and kept at the same dosage throughout the study. Patients will be treated and assessed for 6 weeks

3) Two-Week Follow-Up Period (Day 44 to Day 57)

All randomized patients will be assessed for discontinuation-emergent signs and symptoms (DESS) at 1, 3, 5, 7 and 14 days after their final dose of study medication (Day 44, 46, 48, 50 and 57). No down titration will be needed of sustained release form of quetiapine, anti-depressant medication to be kept at the same dose.

Study Population

Male or female patients, 18 to 65 years old, with a documented clinical diagnosis using the MINI and meeting the DSM-IV of either:

296.2× MDD, Single Episode, or

296.3× MDD, Recurrent

Patients should be on stable treatment with an adequate anti-depressant treatment (sertraline, paroxetine, venlafaxine, citralopram, escitralopram, fluoxetine, bupropion, amitryptyline or duloxetine) at least min dose according to label for 6 weeks including at least one dose increase when permitted according to label to be eligible. Inadequate response is defined as a HAMD score=20 and an HAMD item 1 score=2 at both enrolment and randomization. For verification of HAMD scores a system to systematically review the scores will be set up, as to prevent scale inflation.

An evaluable patient is a patient who received study medications with at least one valid randomization and one post-randomization MADRS assessment.

Study Duration

6 weeks randomized treatment followed by a 2-week follow-up period.

Investigational Products

The eligible patients will be randomly assigned to one of the three treatment arms: sustained release form of quetiapine 150 mg/day, 300 mg/day or placebo as add-on therapy to ongoing anti-depressant treatment.

Tablets to be used in the study are: 50 mg and 300 mg quetiapine sustained release (SR) tablets and placebo tablets to match.

The sustained release form of quetiapine or placebo will be administered once daily at bedtime. All sustained release form of quetiapine patients will start on 50 mg/day, being uptitrated to 150 mg/day at day 3. The patients in the 300 mg/day treatment group will be increased to 300 mg/day on day 5 (see Table 63).

TABLE 63
Titration of investigational product

| Treatment group Day | 150 mg/day | 300 mg/day | placebo |
|---|---|---|---|
| Day 1-2 | 1x 50 mg sustained release | 1x 50 mg sustained release | 3x 50 mg placebo tablets |
| | form of quetiapine tablets | form of quetiapine tablets | 1x 300 mg placebo tablets |
| | 2x 50 mg placebo tablets | 2x 50 mg placebo tablets | |
| | 1x 300 mg placebo tablets | 1x 300 mg placebo tablets | |
| Day 3-4 | 3x 50 mg sustained release | 3x 50 mg sustained release | 3x 50 mg placebo tablets |
| | form of quetiapine tablets | form of quetiapine tablets | 1x 300 mg placebo tablets |
| | 1x 300 mg placebo tablets | 1x 300 mg placebo tablets | |
| Day 5-57 | 3x 50 mg sustained release | 3x 50 mg placebo tablets | 3x 50 mg placebo tablets |
| | form of quetiapine tablets | 1x 300 mg sustained release | 1x 300 mg placebo tablets |
| | 1x 300 mg placebo tablets | form of quetiapine tablets | |

Study Procedures

Eligibility for the study will be assessed at enrolment and randomization. The patients will be randomized to treatment groups at Day 1 after fulfilling all inclusion criteria and none of the exclusion criteria.

Statistical Analysis

Confirmatory Strategy:

Primary objective: The null hypotheses is that there is no difference between the two quetiapine treatments and the placebo treatment in change in MADRS total score from randomization to Week 6. Each quetiapine dose group (150 mg and 300 mg) will be compared to placebo.

Secondary objective of particular interest: The null hypotheses is that there is no difference between the two quetiapine treatments and the placebo treatment in change in Q-LES-Q total score from randomization to Week 6. Each quetiapine dose group (150 mg and 300 mg) will be compared to placebo.

A step-wise sequential testing procedure will be used to handle multiple comparisons across the two groups of hypotheses to ensure that the overall significance level of 0.05 is preserved. First, the primary outcome variable change in MADRS total score from randomization to Week 6 will be tested for each dose versus placebo respectively. If both the quetiapine doses are statistically significantly better than the placebo group, then the hypotheses related to the variable change in Q-LES-Q total score from baseline to Week 6 will be tested for each dose respectively. To handle multiplicity within each step, the Simes-Hommel procedure will be used.

Analysis Populations

The efficacy analyses will be based on the modified intention-to-treat population (Full Analysis Set). This population will include all randomized subjects, classified according to randomized treatment, who took study medication and who have a randomization MADRS assessment and at least 1 valid MADRS assessment after randomization.

The safety displays will be based on the safety population. This population includes all randomized subjects who took study medication, classified according to the treatment actually received.

Primary Efficacy Analysis

The primary outcome variable, the change in MADRS total score from randomization to Week 6, will be analyzed using a mixed model analysis with MADRS total score at randomization as a covariate and including treatment as a fixed effect and centre as a random effect. The comparisons of interest will be the difference between each sustained release form of quetiapine dose and placebo.

Secondary Efficacy Analysis of Primary Interest

The outcome variable, the change in Q-LES-Q total score from randomization to Week 6, will be analyzed using a mixed model analysis with Q-LES-Q total score at randomization as a covariate and including treatment as a fixed effect and centre as a random effect. The comparisons of interest will be the difference between each sustained release form of quetiapine dose and placebo.

Rationale for Sample Size

The sample size calculation in this study was done to ensure an 80% power in demonstrating superior efficacy of each of the two sustained release form of quetiapine doses over placebo with respect to the primary outcome variable, change in MADRS total score from baseline to Week 6. Then, the appropriate sample size was attained by assuming an anticipated difference of 3.5 units from placebo and a within patient variability (standard deviation) of 9 for the change in MADRAS total score from baseline to Week 6. Using a two-sided test at a 5% significance level, this yields a planned sample size of 140/arm, and 420 in total to ensure a power of 90% in each individual comparison and an overall power of at least 80%. Assuming that 93% of all randomized patients are expected to be evaluable patients (to be included in MITT), a total of about 450 randomized patients are required to obtain 140 evaluable patients per treatment group.

TABLE 64
Sample size calculation

| | As specified |
|---|---|
| Over all Power/individual power | 80%/90% |
| Difference to be detected | 3.5 |
| compared to placebo | |
| Standard deviation | 9 |
| Overall Significance level | 0.05 |
| Sample size (evaluable) | 140 |

Inclusion/Exclusion Criteria, Restrictions and Rationale

TABLE 65
MDD Inclusion criteria

| MDD Inclusion Criteria | | Rationale |
|---|---|---|
| 1. | Men and women aged 18 to 65 years. | To include adult patients |
| | | but exclude the elderly |
| | | population. |
| 2. | Documented clinical diagnosis meeting | |
| | criteria from the Diagnostic and Statistical | |
| | Manual of Mental Disorders, 4^{th} edition | |
| | (DSM-IV) for any of the following: | |
| | 296.2x MDD, Single Episode, or | |
| | 296.3x MDD, Recurrent | |
| 3. | HAM-D (17-item) total score of = 22 and | To ensure that patients |
| | HAM-D item 1 (depressed mood) | have a moderate to |
| | score = 2 both at enrolment (visit 1) and | severe degree of |
| | randomization (Visit 2). | depression and to ensure |
| | | that the patients are still |
| | | eligible at randomization |
| 4. | History of in-adequate response to an | To include patients with |
| | adequate anti-depressant treatment | inadequate response |
| | (sertraline, paroxetine, venlafaxine, | |
| | citralopram, escitralopram, fluoxetine, | |
| | bupropion, amitryptyline or duloxetine) at | |
| | least min dose according to label for 6 | |
| | weeks including at least one dose increase | |
| | when permitted according to label and | |
| | tolerability during current depressive | |
| | episode | |

TABLE 66
MDD Exclusion criteria

| MDD Exclusion Criteria | | Rationale |
|---|---|---|
| 1. | Patients with a DSM-IV Axis I disorder other | To exclude other diagnoses which may |
| | than MDD within 6 months of enrolment. | confound results |
| | 2. Patients whose current episode of depression | To exclude treatment-resistant patients and |
| | exceeds 12 months or is less than 4 weeks from | patients with incorrect diagnoses. |
| | enrolment. | |
| 3. | Substance or alcohol abuse or dependence | To exclude patients with active substance abuse, |
| | within 6 months prior to screening as defined in | which may interfere with assessments of mood |
| | DSM-IV criteria | |
| 4. | Use of drugs that induce or inhibit the hepatic | To ensure more consistent levels of study drug |
| | metabolizing cytochrome 3A4 enzymes within | across patient populations. |
| | 2 weeks prior to randomization (e.g. inducers: | |
| | carbamazepine, phenytoin, barbiturates, | |
| | rifampin, rifabutin, glucocorticoids, thioridazine | |
| | and St John's wort, and inhibitors: ketoconazole | |
| | (except for topical use), itraconazole, | |
| | fluconazole, erythromycin, clarithromycin, | |
| | fluvoxamine, nefazodone, troleandomycin, | |
| | indinavir, nelfinavir, ritonavir, and saquinavir). | |
| 5. | Use of antipsychotic or mood stabilizer other | To ensure that previous psychotropic drugs do |
| | than allowed, or other psychoactive drugs | not affect study assessments. |
| | within 7 days before randomization, or use of | |
| | MAO inhibitors anxiolytic drugs or hypnotics | |
| | within 14 days before randomization, or use of a | |
| | depot antipsychotic injection within two dosing | |
| | interval before randomization. | |

TABLE 67
Restrictions in treatments

| Restrictions | Rationale |
|---|---|
| Prohibited | |
| Use of drugs that induce or inhibit the hepatic | Potentially |
| metabolizing cytochrome 3A4 enzymes within 2 | confounds the |
| weeks prior to randomization (e.g. inducers: | results. |
| carbamazepine, phenytoin, barbiturates, rifampin, | To ensure safety. |
| rifabutin, glucocorticoids, thioridazine and St John's | |
| wort, and inhibitors: ketoconazole (except for | |
| topical use), itraconazole, fluconazole, erytromycin, | |
| clarithomycin, fluvoxamine, nefazodone, | |
| troleandomycin, indinavir, nelfinavir, ritonavir, and | |
| saquinavir). | |
| Use of any psychoactive drugs including | Potentially |
| antidepressant, anxiolytic, hypnotic, mood | confounds the |
| stabilizing, antipsychotic, and sedative medications | results. |
| other than restricted. | |
| One anti-depressant that has been ongoing for 6 | To have patients |
| weeks prior enrolment. Dosage should be stable at | with one |
| enrolment and remain at the same dose throughout | anti-depressant |
| the study | treatment in |
| | the study |
| Permitted | |
| Treatment with one of the following anti depressants | Patients should be |
| during the study period at the same dose as when | on adjunct therapy |
| entering the study: | and with and anti- |
| | depressant. |

### Example 22

### Treatment of Patients with MDD

This study is a 6-week multicentre, double-blind, randomized, parallel-group, placebo-controlled Phase III Study of the efficacy and safety of a sustained release form of quetiapine fumarate 150 mg/day and 300 mg/day in combination with an anti-depressant in the treatment of patients with MDD with inadequate response to an antidepressant treatment. The objective for this study is to evaluate that a sustained release form of quetiapine in combination with an anti-depressant is efficacious and safe in the treatment of MDD in patients who have inadequate response to an antidepressant. The primary objective is described in Table 65 and the secondary objectives of the study are shown in Table 69.

TABLE 68
Primary objective, corresponding outcome variables and claims

| Claims to be addressed | Primary Objective | Outcome Variable |
|---|---|---|
| 2.2 A sustained release form of | To evaluate the efficacy of | Primary variable: |
| quetiapine once daily in | sustained release form of | Change from randomization to Week 6 |
| combination with an anti- | quetiapine in combination with | in the Montgomery-Åsberg Depression |
| depressant has superior efficacy | an anti-depressant versus an anti- | Rating Scale (MADRS) total score |
| to an anti-depressant alone in | depressant alone in patients with | Secondary variables supporting the |
| depression | MDD. | primary objective: |
| 2.4 A sustained release form of | | Change from randomization to each |
| quetiapine once daily in | | assessment in the MADRS total score |
| combination with an anti- | | MADRS response, defined as a = 50% |
| depressant has a greater | | reduction from randomization in the |
| response rate than an anti- | | MADRS total score at Week 6. |
| depressant alone in depression | | MADRS remission, defined as total |
| 2.6 A sustained release form of | | score ≦8 at Week 6. |
| quetiapine once daily in | | Change from randomization to week 6 |
| combination with an anti- | | in the Hamilton Depression scale |
| depressant is better than an anti- | | (HAM-D) total score and the HAM-D |
| depressant alone in achieving | | Item 1. |
| remission in patients with | | Change from randomization to each |
| depression | | assessment in the Clinical Global |
| 9.1 Starting on day 1 A | | Impression - Severity (CGI-S) |
| sustained release form of | | Clinical Global Impression - |
| quetiapine once daily in | | Improvement (CGI-I) at each |
| combination with an anti- | | assessment |
| depressant can be prescribed at | | |
| a therapeutically effective dose | | |
| in depression | | |

TABLE 69
Secondary objectives, corresponding outcome variables and claims

| Claims to be addressed or | | |
|---|---|---|
| hypothesis to be tested | Secondary Objective | Outcome Variables |
| Efficacy | | |
| 3.2 A sustained release form of | To evaluate if sustained release | Change from randomization to each |
| quetiapine once daily in | form of quetiapine in | assessment in Hamilton Rating scale for |
| combination with an anti- | combination with an anti- | Anxiety (HAM-A) |
| depressant is more effective | depressant reduces anxiety | Change in HAM-A psychic anxiety |
| than an anti-depressant alone in | symptoms in patients with MDD, | factors (Anxious Mood, Tension, Fears, |
| reducing anxiety symptoms in | compared to an anti-depressant | Insomnia, Intellect, Depressed Mood, |
| depression | alone. | Behaviour at interview) from |
| | | randomization to each assessment |
| | | Change in HAM-D anxiety factors |
| | | (item 10 and 11) from randomization to |
| | | Week 6. |
| 4.2 A sustained release form of | To evaluate if sustained release | Change in HAM-D sleep disturbance |
| quetiapine once daily in | form of quetiapine in | factors (Items 4-6) from randomization |
| combination with an anti- | combination with an anti- | to Week 6 |
| depressant is more effective | depressant improves sleep quality | Change in Pittsburgh Sleep Quality |
| than an anti-depressant alone in | in patients with MDD, compared | Index (PSQI) global score from |
| improving sleep onset and sleep | to an anti-depressant alone. | randomization to each assessment |
| maintenance in depression | | |
| 5.2 A sustained release form of | To evaluate if sustained release | Change from randomization to each |
| quetiapine once daily in | form of quetiapine in | assessment in MADRS item 10, suicidal |
| combination with an anti- | combination with an anti- | thought |
| depressant is more effective | depressant is effective in | |
| than an anti-depressant alone in | reducing suicidal ideation in | |
| reducing suicide ideation in | patients with MDD, compared to | |
| patients with depression | an anti-depressant alone. | |
| 8.2 A sustained release form of | To evaluate if sustained release | Change in HAM-A somatic anxiety |
| quetiapine once daily in | form of quetiapine in | factors (Somatic Muscular, Somatic |
| combination with an anti- | combination with an anti- | Sensory, Cardiovascular, Respiratory, |
| depressant is more effective | depressant improves somatic | Gastrointestinal, Genitourinary, |
| than an anti-depressant alone in | symptoms in the treatment of | Autonomic) from randomization to |
| improving somatic symptoms | patients with MDD, compared to | each assessment. |
| such as back pain, headache, | an anti-depressant alone.. | |
| muscle pain, unspecified pain, | | |
| abdominal pain, chest pain, in | | |
| patients with depression | | |
| Efficacy-Quality of Life | | |
| 6.2 A sustained release form of | To evaluate if sustained release | Change from baseline to each |
| quetiapine once daily in | form of quetiapine in | assessment in Q-les-Q total score (item |
| combination with an anti- | combination with an anti- | 1-14). |
| depressant is more effective | depressant improves the quality | Change from baseline to each |
| than an anti-depressant alone in | of life of patients with MDD, | assessment in Q-les-Q Item 16 (Overall |
| improving the quality of life of | compared to an anti-depressant | quality of life). |
| patients with depression | alone. | |
| 7.2 A sustained release form of | To evaluate if sustained release | Change from randomization to each |
| quetiapine once daily in | form of quetiapine in | assessment in Q-les-Q Item 15 |
| combination with an anti- | combination with an anti- | (Satisfaction with medication) |
| depressant is more effective | depressant improves patient | |
| than an anti-depressant alone in | satisfaction in patients with | |
| improving patient satisfaction in | MDD, compared to an anti- | |
| patients with depression | depressant alone. | |
| Safety/tolerability | | |
| 11.1 A sustained release form of | To evaluate if sustained release | Change from normal to Clinically |
| quetiapine once daily in | form of quetiapine in | Important in: |
| combination with an anti- | combination with an anti- | Physical Examinations |
| depressant up to 300 mg/d is | depressant is safe and well- | Laboratory values (including |
| well tolerated in patients with | tolerated in the treatment of | glucose/lipids) |
| depression | patients with MDD. | Vital signs |
| 11.10 Fasting Glucose and | | Electrocardiograms (ECGs) |
| Lipids not significantly elevated | | Adverse Events |
| 11.8 A sustained release form of | | AEs leading to withdrawal |
| quetiapine once daily in | | AEs related to somnolence |
| combination with an anti- | | Severity of somnolence reports |
| depressant does not have | | Time of AE somnolence reports |
| serious discontinuation | | Withdrawals due to AE of somnolence |
| symptoms | | Change in weight from randomization |
| 11.9 Somnolence with A | | to each assessment |
| sustained release form of | | Change in waist circumference from |
| quetiapine once daily in | | randomization to each assessment |
| combination with an anti- | | Proportion of patients with a ≧7% |
| depressant is generally mild, | | increase from randomization weight. |
| occurs early in treatment; is not | | |
| persistent in the majority of | | |
| patients and is rarely a cause of | | |
| withdrawal | | |
| 11.13 A sustained release form | | |
| of quetiapine once daily in | | |
| combination with an anti- | | |
| depressant is associated with a | | |
| favourable weight profile | | |
| 11.2 A sustained release form of | For 11.2, 11.4, 11.6, and 11.12 | AEs (especially related to sexual |
| quetiapine once daily in | To evaluate if sustained release | dysfunction, nausea, vomiting, EPS |
| combination with an anti- | form of quetiapine in | including akathisia) |
| depressant is associated with | combination with an anti- | Change from randomization to each |
| levels of sexual dysfunction as | depressant is as safe and well- | assessment in Sexual Functioning |
| an anti-depressant alone | tolerated as an anti-depressant | Questionnaire (CSFQ) total score |
| 11.4 A sustained release form of | alone in the treatment of patients | Change in SAS and BARS from |
| quetiapine once daily in | with MDD | randomization to each assessment |
| combination with an anti- | | MADRS item 10 score = 4 at any time |
| depressant is associated with | | after randomization or AE of |
| levels of nausea and vomiting | | suicidality/suicidal ideation/suicide |
| as an anti-depressant alone | | attempts/suicide completion |
| 11.6 A sustained release form of | | Analysis of suicidality according to |
| quetiapine once daily in | | FDA guidance <<(tbc)>> |
| combination with an anti- | | |
| depressant is associated with | | |
| levels of EPS (including | | |
| akathisia) as an anti-depressant | | |
| alone | | |
| 11.12 A sustained release form | | |
| of quetiapine once daily in | | |
| combination with an anti- | | |
| depressant is associated with a | | |
| lower incidence of treatment | | |
| emergent suicidal ideation | | |
| compared with an anti- | | |
| depressant alone | | |
| Pharmacokinetics | | |
| — | To evaluate if sustained release | Change in concentration of |
| | form of quetiapine in | antidepressant from randomization to |
| | combination with an | Week 4 |
| | antidepressant changes the level | |
| | of antidepressant in the | |
| | circulation | |

This is a 6-week Randomized, Parallel-group, Placebo-controlled Study of the Efficacy and Safety of quetiapine Fumarate (SEROQUEL®) in combination with an anti-depressant in the Treatment of Patients with MDD with inadequate response to an antidepressant treatment.

The study comprises the following two periods:

1) Washout Period

Patients shall be evaluated and meet the DSM-IV diagnosis confirmed by the MINI and will undergo enrolment assessments at Visit 1, eligible patients will commence a washout during which prohibited medication should be washed out prior to randomization. For verification of Patients should be on stable treatment with an adequate anti-depressant treatment (sertraline, paroxetine, venlafaxine, citralopram, escitralopram, fluoxetine, bupropion, amitryptyline or duloxetine) at least min dose according to label for 6 weeks including at least one dose increase when permitted according to label as well as having a HAMD score of at least 20 to be eligible. For verification of HAMD scores a system to systematically review the scores will be set up, as to prevent scale inflation.

Six-Week Double-Blind, Randomized, Placebo Controlled Treatment Period (Day 1 to Day 43)

Eligible patients will be randomized on Day 1 (Visit 2) to one of three treatment groups: sustained release form of quetiapine 150 mg/day, sustained release form of quetiapine 300 mg/day or placebo as add-on therapy to ongoing anti-depressant treatment. The ongoing treatment with the anti-depressant should be stable when entering the study and kept at the same dosage throughout the study.

Patients will be treated and assessed for 6 weeks according to schedule below.

Hospitalization is allowed, if deemed necessary for the safety and well-being of the patient, up to 2 weeks after randomization as judged by the investigator.

Study Population

Male or female patients, 18 to 65 years old, with a documented clinical diagnosis using the MINI and meeting the DSM-IV of either:

296.2× MDD, Single Episode, or

296.3× MDD, Recurrent

Patients should be on stable treatment with an adequate anti-depressant treatment (sertraline, paroxetine, venlafaxine, citralopram, escitralopram, fluoxetine, bupropion, amitryptyline or duloxetine) at least min dose according to label for 6 weeks including at least one dose increase when permitted according to label to be eligible. Inadequate response is defined as a HAMD score=20 and a HAMD item 1 score=2 at both enrolment and randomization. For verification of HAMD scores a system to systematically review the scores will be set up, as to prevent scale inflation.

Number of Patients

About 450 patients, will be randomized at approximately 23 centres, with about 20 patients per center, to obtain 140 evaluable patients (7% attrition rate) per treatment group (sustained release form of quetiapine and placebo arms) in a 1:1:1 randomization. An evaluable patient is a patient who received study medications with at least one valid randomization and one post-randomization MADRS assessment.

Study Duration

6 weeks randomized treatment.

Investigational Products

The eligible patients will be randomly assigned to one of the three treatment arms: sustained release form of quetiapine 150 mg/day, 300 mg/day or placebo as add-on therapy to ongoing anti-depressant treatment.

Tablets to be used in the study are: 50 mg and 300 mg quetiapine sustained release (SR) tablets and placebo tablets to match.

The sustained release form of quetiapine or placebo will be administered once daily at bedtime. All sustained release form of quetiapine patients will start on 50 mg/day, being uptitrated to 150 mg/day at day 3. The patients in the 300 mg/day treatment group will be increased to 300 mg/day on day 5.

TABLE 70
Titration of investigational product

| Treatment group Day | 150 mg/day | 300 mg/day | placebo |
|---|---|---|---|
| Day 1-2 | 1x 50 mg sustained release | 1x 50 mg sustained release | 3x 50 mg placebo tablets |
| | form of quetiapine tablets | form of quetiapine tablets | 1x 300 mg placebo tablets |
| | 2x 50 mg placebo tablets | 2x 50 mg placebo tablets | |
| | 1x 300 mg placebo tablets | 1x 300 mg placebo tablets | |
| Day 3-4 | 3x 50 mg sustained release | 3x 50 mg sustained release | 3x 50 mg placebo tablets |
| | form of quetiapine tablets | form of quetiapine tablets | 1x 300 mg placebo tablets |
| | 1x 300 mg placebo tablets | 1x 300 mg placebo tablets | |
| Day 5-57 | 3x 50 mg sustained release | 3x 50 mg placebo tablets | 3x 50 mg placebo tablets |
| | form of quetiapine tablets | 1x 300 mg sustained release | 1x 300 mg placebo tablets |
| | 1x 300 mg placebo tablets | form of quetiapine tablets | |

Study Procedures

Eligibility for the study will be assessed at enrolment and randomization. The patients will be randomized to treatment groups at Day 1 after fulfilling all inclusion criteria and none of the exclusion criteria. All visits allow a visit window of ±2 days calculated from randomization.

Statistical Analysis

Confirmatory Strategy:

Primary objective: The null hypotheses is that there is no difference between the two quetiapine treatments and the placebo treatment in change in MADRS total score from randomization to Week 6. Each quetiapine dose group (150 mg and 300 mg) will be compared to placebo.

Secondary objective of particular interest: The null hypotheses is that there is no difference between the two quetiapine treatments and the placebo treatment in change in Q-LES-Q total score from randomization to Week 6. Each quetiapine dose group (150 mg and 300 mg) will be compared to placebo.

A step-wise sequential testing procedure will be used to handle multiple comparisons across the two groups of hypotheses to ensure that the overall significance level of 0.05 is preserved. First, the primary outcome variable change in MADRS total score from randomization to Week 6 will be tested for each dose versus placebo respectively. If both the quetiapine doses are statistically significantly better than the placebo group, then the hypotheses related to the variable change in Q-LES-Q total score from baseline to Week 6 will be tested for each dose respectively. To handle multiplicity within each step, the Simes-Hommel procedure will be used.

Analysis Populations

The efficacy analyses will be based on the modified intention-to-treat population (Full Analysis Set). This population will include all randomized subjects, classified according to randomized treatment, who took study medication and who have a randomization MADRS assessment and at least 1 valid MADRS assessment after randomization.

The safety displays will be based on the safety population. This population includes all randomized subjects who took study medication, classified according to the treatment actually received.

Primary Efficacy Analysis

The primary outcome variable, the change in MADRS total score from randomization to Week 6, will be analyzed using a mixed model analysis with MADRS total score at randomization as a covariate and including treatment as a fixed effect and centre as a random effect. The comparisons of interest will be the difference between each sustained release form of quetiapine dose and placebo.

Secondary Efficacy Analysis of Primary Interest

The outcome variable, the change in Q-LES-Q total score from randomization to Week 6, will be analyzed using a mixed model analysis with Q-LES-Q total score at randomization as a covariate and including treatment as a fixed effect and centre as a random effect. The comparisons of interest will be the difference between each sustained release form of quetiapine dose and placebo.

Rationale FOR Sample Size

The sample size calculation in this study was done to ensure an 80% power in demonstrating superior efficacy of each of the two sustained release form of quetiapine doses over placebo with regard to the primary outcome variable, change in MADRS total score from baseline to Week 6. Then, the appropriate sample size was attained by assuming an anticipated difference of 3.5 units from placebo and a within patient variability (standard deviation) of 9 for the change in MADRAS total score from baseline to Week 6. Using a two-sided test at a 5% significance level, this yields a planned sample size of 140/arm, and 420 in total to ensure a power of 90% in each individual comparison and an overall power of at least 80%.

Assuming that 93% of all randomized patients are expected to be evaluable patients (to be included in MITT), a total of about 450 randomized patients are required to obtain 140 evaluable patients per treatment group.

TABLE 71
Sample size calculation

| | As specified |
|---|---|
| Over all Power/individual | 80%/90% |
| power | |
| Difference to be detected | 3.5 |
| compared to placebo | |
| Standard deviation | 9 |
| Overall Significance level | 0.05 |
| Sample size (evaluable) | 140 |

Inclusion/Exclusion Criteria, Restrictions and Rationale

TABLE 72
MDD Inclusion criteria

| MDD Inclusion Criteria | | Rationale |
|---|---|---|
| 1. | Men and women aged 18 to 65 years. | To include adult patients but exclude the elderly |
| | | population. |
| 2. | Documented clinical diagnosis meeting criteria | |
| | from the Diagnostic and Statistical Manual of | |
| | Mental Disorders, 4^{th} edition (DSM-IV) for any of | |
| | the following: | |
| | 296.2x MDD, Single Episode, or | |
| | 296.3x MDD, Recurrent | |
| 3. | HAM-D (17-item) total score of = 20 and HAM-D | To ensure that patients have a moderate to |
| | item 1 (depressed mood) score = 2 both at | severe degree of depression and to ensure that |
| | enrolment (visit 1) and randomization (Visit 2). | the patients are still eligible at randomization |
| 4. | History of in-adequate response to an adequate | To include patients with inadequate response |
| | anti-depressant treatment (sertraline, paroxetine, | |
| | venlafaxine, citralopram, escitralopram, fluoxetine, | |
| | bupropion, amitryptyline or duloxetine) at least min | |
| | dose according to label for 6 weeks including at | |
| | least one dose increase when permitted according | |
| | to label and tolerability during current depressive | |
| | episode | |

TABLE 73
MDD Exclusion criteria

| MDD Exclusion Criteria | | Rationale |
|---|---|---|
| 1. | Patients with a DSM-IV Axis I disorder other than | To exclude other diagnoses which may |
| | MDD within 6 months of enrolment. | confound results |
| 2. | Patients whose current episode of depression | To exclude treatment-resistant patients and |
| | exceeds 12 months or is less than 4 weeks from | patients with incorrect diagnoses. |
| | enrolment. | |
| 3. | Substance or alcohol abuse or dependence within 6 | To exclude patients with active substance abuse, |
| | months prior to screening (except dependence in | which may interfere with assessments of mood |
| | full remission, and except for caffeine or nicotine | |
| | dependence), as defined in DSM-IV criteria. | |
| 4. | Use of drugs that induce or inhibit the hepatic | To ensure more consistent levels of study drug |
| | metabolizing cytochrome 3A4 enzymes within 2 | across patient populations. |
| | weeks prior to randomization (e.g. inducers: | |
| | carbamazepine, phenytoin, barbiturates, rifampin, | |
| | rifabutin, glucocorticoids, thioridazine and St | |
| | John's wort, and inhibitors: ketoconazole (except | |
| | for topical use), itraconazole, fluconazole, | |
| | erythromycin, clarithromycin, fluvoxamine, | |
| | nefazodone, troleandomycin, indinavir, nelfinavir, | |
| | ritonavir, and saquinavir). | |
| 5. | Use of antipsychotic or mood stabilizer other than | To ensure that previous psychotropic drugs do |
| | allowed, or other psychoactive drugs within 7 days | not affect study assessments. |
| | before randomization, or use of MAO inhibitors | |
| | anxiolytic drugs or hypnotics within 14 days before | |
| | randomization, or use of a depot antipsychotic | |
| | injection within two dosing interval before | |
| | randomization. | |

TABLE 74
Restrictions in treatments

| Restrictions | Rationale |
|---|---|
| Prohibited | |
| Use of drugs that induce or inhibit the hepatic | Potentially confounds the results. |
| metabolizing cytochrome 3A4 enzymes within 2 | To ensure safety. |
| weeks prior to randomization (e.g. inducers: | |
| carbamazepine, phenytoin, barbiturates, rifampin, | |
| rifabutin, glucocorticoids, thioridazine and St John's | |
| wort, and inhibitors: ketoconazole (except for topical | |
| use), itraconazole, fluconazole, erytromycin, | |
| clarithomycin, fluvoxamine, nefazodone, | |
| troleandomycin, indinavir, nelfinavir, ritonavir, and | |
| saquinavir). | |
| Use of any psychoactive drugs including | Potentially confounds the results. |
| antidepressant, anxiolytic, hypnotic, mood stabilizing, | |
| antipsychotic, and sedative medications other than | |
| restricted. | |
| Electroconvulsive therapy (ECT) throughout the | Potentially confounds the results. |
| randomized treatment period. | |
| Abuse according to the DSM-IV criteria of Alcohol, | Potentially confounds the results. |
| Opiates, amphetamine, barbiturate, cocaine, cannabis, | |
| or hallucinogen throughout the study | |
| Restricted | |
| One of the following can be used for insomnia (at | Need to allow hypnotics at reasonable doses. |
| bedtime) up to the specified dosage per night if | Differences in treatment traditions and availability of |
| treatment has been ongoing since 30 days prior | products between countries require alternative |
| enrolment on a regular basis as judged by the | products. |
| investigator: | |
| lorazepam max 2 mg/day | |
| zolpidem tartrate 10 mg; | |
| zaleplon, 20 mg; | |
| zopiclone, 7.5 mg; | |
| chloral hydrate, 1 g. | |
| Hypnotic use not allowed on the night prior to | |
| conducting study assessments. | |
| Anticholinergics can be used to treat extrapyramidal | Need to allow anticholinergics for treatment of EPS |
| symptoms (EPS). Prophylactic use of anticholinergics | but not prophylactic as it could potentially mask |
| is prohibited. | extrapyramidal symptoms. |
| Psychotherapy is only allowed if it has been ongoing | |
| since at least 3 months prior to randomization. | |
| One anti-depressant that has been ongoing for 6 | To have patients with one anti-depressant treatment in |
| weeks prior enrolment. Dosage should be stable at | the study |
| enrolment and remain at the same dose throughout | |
| the study | |
| Permitted | |
| Treatment with one of the following anti depressants | Patients should be on adjunct therapy with and anti- |
| during the study period at the same dose as when | depressant. |
| entering the study: | |
| sertraline | |
| paroxetine | |
| venlafaxine | |
| citralopram | |
| escitralopram | |
| fluoxetine | |
| bupropion | |
| amitryptyline | |
| duloxetine | |
| Nonpsychoactive medications, including over-the- | patients may require medications to treat underlying |
| counter medications which are required to treat | medical conditions |
| illness or complaints that occur during the study | |
| Other medications which are considered necessary for | At the discretion of the investigator, patients may |
| the patient's safety and well-being | require medications and/or devices, eg. for |
| | contraception |

### Example 23

### Treatment of GAD

GAD is a chronic condition which affects approximately 5% of individuals in the community. It imposes a substantial impact on individual and global economies because of its resulting morbidity and disability. GAD commonly presents in patients who suffer from other psychiatric disorders and/or medical conditions including, but not limited to: 1) major depression; 2) other anxiety disorders; 3) cardiovascular, gastrointestinal, and respiratory disease.

GAD is generally treated with SSRIs, SNRIs, and benzodiazepines. However, only one third of the patients treated with these medications achieve remission within one year. Furthermore, even those patients who realize some benefit initially often suffer relapse. In addition, SSRIs and SNRIs are associated with nausea and sexual dysfunction, while the risk of dependence associated with benzodiazepines reduces their appeal as a long-term solution.

Quetiapine is an atypical antipsychotic which has proved efficacious in reducing the symptoms of anxiety and depression in patients with schizophrenia and schizoaffective disorder. It has also demonstrated the ability to reduce Hamilton Anxiety Rating Scale (HAM-A) scores in clinical trials of patients with bipolar depression.

Population and Protocol

The tolerability and efficacy of quetiapine as adjunctive treatment to conventional medication in patients with treatment resistant or non-remitted GAD was assessed in a 12-week, open label, flexible dose study. The study population was limited to GAD patients who had not remitted following at least 8 weeks of therapeutic doses of conventional therapy. Conventional therapy includes treatment with one of the following: citalopram, escitalopram, paroxetine, paroxetine CR, venlafaxine XR, sertraline, mirtazapine, fluoxetine, fluvoxamine, or no medication at all. Each patient provided informed consent and was observed on an outpatient basis. Of the 50 patients eligible for inclusion in the study, 32 completed it. Eight patients withdrew consent, seven withdrew due to adverse events, two were lost during follow-up, and one was excluded for violating study protocol.

The study population was comprised of patients between the ages of 22 and 61. Forty-eight percent of the study population was male and fifty-two percent was female. Patients each had a primary diagnosis of GAD (DSM-IV), a Clinical Impressions-Severity of Illness (CGI-S) score of 5-7, a HAM-A total score ≧20 and a score of ≧2 on the two HAM-A subscales for anxious mood and tension. Patients with other psychiatric disorders, subjective suicidal tendencies, a Montgomery-Asberg Depression Rating Scale (MADRS) score>19, or serious medical conditions were excluded from the study. In addition, pregnant or lactating women and those who took medications prohibited by the study design (e.g., psychoactive substances and oral antipsychotics) within 2 weeks of the original screening were also excluded.

Patients received fixed doses of one of the conventional therapies listed above and were also given an initial dose of 25 mg/day of quetiapine once daily. The dose of quetiapine was titrated on a weekly basis in increments of 25-100 mg/day once daily at the clinician's discretion. The maximum dose of quetiapine was 800 mg/day and the dose taken at Week 9 was fixed for the remainder of the study. The mean dose for those who completed the study was 386 mg/day.

Results

The mean change from baseline to Week 12 in HAM-A total scores was the primary endpoint. The addition of quetiapine to conventional therapy reduced HAM-A total scores from about 30 to about 9 after 12 weeks of treatment. More than 72% of patients achieved the secondary endpoint of remission at Week 12. In addition to the primary and secondary endpoints, assessments were made concerning the efficacy of quetiapine on: (1) sleep quality and attitude; (2) general disability in terms of work, family, and social interactions; and (3) patient worries. These additional efficacy assessments were all measured from baseline to Week 12. The sleep quality for study patients, as measured by the PSQI, improved from a baseline mean of about 17 to a mean of about 7 in Week 12. The patients also recognized an improvement in their attitudes towards sleep, demonstrated by a the change from a mean of about 88 to a mean of about 73 on the Dysfunctional Beliefs and Attitudes about Sleep Scale (DBAS). Patients also demonstrated an improvement in the all three subscales of the Sheehan Disability Inventory (SDI). Mean SDI scores improved by about 2 in terms of work, family, and social interactions. Adjunct quetiapine also improved patient worry, as measured by the Penn State Worry Questionnaire (PSWQ), from a mean of about 53 to a mean of about 44.

Tolerability & Adverse Events

Of the patients who withdrew as a result of adverse events, five withdrew because of sedation, 1 withdrew because of a panic attack, and 1 withdrew due to a non-treatment related case of bilateral iritis. Forty percent of patients who entered the study reported sedation as an adverse event, but no serious adverse events were reported.

There were slight increases in both mean total weight gain and BMI from baseline to Week 12, however, no clinically significant changes in vital signs or laboratory assessments were reported.

The results of this study indicate that quetiapine is effective as adjunct therapy in patients with treatment-resistant or non-remitted GAD as demonstrated by clinically significant improvements in both primary and secondary endpoints. Its efficacy is also illustrated by the fact that 72.5% of study patients achieved remission by study end.

### Example 24

### Quetiapine v. Lithium

Resistance to anti-depressant treatment occurs in 30-50% of patients treated; rates of remission are even lower. Lithium is the most extensively studied medication in its use as an adjunct to anti-depressant therapy. Studies have shown that almost 50% of patients respond to lithium within 4 weeks.

This study compared augmentation of antidepressant therapy with quetiapine versus augmentation with lithium in patients with treatment resistant major depression. Patients were given either quetiapine or lithium in addition to their existing antidepressant regimen. The study compared tolerability and efficacy. Quetiapine was increased in 50-75 mg increments to 400 mg/day in the first week and subsequently adjusted to a maximum of 800 mg/day. Lithium was initiated at 600 mg/day, and maintained over Week 1 and 2 and subsequently adjusted to attain a level of 0.8-1.2 mmol/L. Lithium levels were taken weekly. Mean doses for quetiapine and lithium were 430 mg/day and 825 mg/day respectively.

A total of 20 patients were randomized to 1 of the 2 treatment groups. The study population included men and women aged 18-65 years, with a diagnosis of major depression according to DSM-IV, Hamilton Depression Rating Scale (HAM-D)≧20 and a Clinical Global Inspection (CGI) score≧4. Patients also had to have shown a resistance to a maximal dose of antidepressant treatment after a minimum of 4 weeks. Those patients who exhibited psychotic symptoms, had a history of bipolar affective disorder I or II, a substance use disorder within the past 6 months, or suffered from an unstable medical condition were excluded.

Patients were evaluated at baseline and then on days 7, 14, 28, 42, and 56 using the HAM-D, Montgomery-Asberg Rating Scale (MADRS), SAS, and Widlocher Psychomotor Retardation Scale.

Results

Depression as measured by HAM-D significantly improved in both treatment groups over 56 days. However, from Day 7 to 56, improvements with quetiapine were linear, while improvement remained relatively unchanged with lithium. From Day 7 to 56, the mean HAM-D score for quetiapine improved from about 26 to about 5, while the mean score for lithium only improved from about 28 to about 15. In addition, about 80% of patients in the quetiapine group responded to treatment and were in remission after 56 days, while only 50% of the patients on lithium responded and only 40% of them achieved remission. Results similar to those obtained in the HAM-D assessment were obtained with the MADRS evaluation, i.e., linear improvement with quetiapine continued after Day 7 but did not with lithium. After Day 7, the mean MADRS score for quetiapine improved from about 3 8 to about 8, while the mean score for lithium remained at about 22. Response and remission rates based on the MADRS evaluation were also similar to the response and remission rates observed in the HAM-D assessment. Eighty-percent of patients in the quetiapine group responded and achieved remission after 56 days, while only 50% of patients in the lithium group responded and only 30% achieved remission at 56 days. Similar results were also obtained when patients were assessed using the Widlocher Psychomotor Retardation Scale, where both groups showed improvement until Day 7 and only the quetiapine group showed subsequent improvement. The quetiapine group improved from a mean baseline score of about 30 to about 7, while the lithium group improved from a mean baseline score of about 22 to about 15. In addition to showing greater improvement in scores according to 3 different evaluation scales, patients in the quetiapine group showed decreased scores from baseline as measured by the SAS scale, while patients in the lithium group exhibited increased scores from baseline on the SAS scale.

Although both the quetiapine and lithium groups demonstrated improvement in response to treatment, the improvement for the quetiapine group continued after Day 7.

### Example 25

### Quetiapine as Mono-Therapy for GAD

Quetiapine has demonstrated potential efficacy as adjunct therapy as agents in treatment-resistant GAD. This study involved a double-blind, placebo controlled trial to assess the efficacy and tolerability of quetiapine monotherapy in patients suffering from GAD.

Thirty-eight (19 in each group), non-depressed patients with (GAD) were randomized, following a one-week placebo run-in, to 6 weeks of double-blind treatment with quetiapine or placebo. Patients were assessed at Weeks 1, 2, 4, and 6. There were 12 males in the quetiapine group and 7 males in the placebo group. The mean age of each group was about 40 and the mean age of onset of GAD was about 30 for both groups. Both groups had similar baseline HAM-A total scores of about 23 and mean baseline HAM-A psychic anxiety subscale scores of about 14. The mean baseline CGI-S score for both groups was about 4 and the mean baseline HAD-anxiety score for both groups was about 12. Efficacy was measured primarily by the change in Hamilton Anxiety Rating Scale (HAM-A) total score from baseline to week 6. In addition, response (≧50% reduction in HAM-A total score) and remission (HAM-A total score≦7) rates were also assessed. Safety assessments were made based upon the Abnormal Involuntary Movement Scale (AIMS), SAS, and BARS. Other adverse events (AEs) were also monitored.

In order to qualify for the study patients had to be at least 18 years of age and have: (1) a primary diagnosis of DSM-IV GAD as assessed by the Mimi-International Neuropsychiatric Interview (MINI); (2) a HAM-A total score≧20 (≧2 on anxious mood and tension items); and (3) a CGI-S score≧4. Women of childbearing age were allowed, provided that the accepted methods of contraception were used. The mean dose of quetiapine at trial end was 125 mg/day. The maximum daily dose given to a patient was 300 mg/day.

Applicants who suffered from depression or more than 3 panic attacks within the month prior to screening were excluded. Those patients with a history or presence of a psychotic disorder or bipolar disorder were also excluded. In addition, those who met the DSM-IV criteria for substance abuse within 6 months prior to screening or were being treated with antidepressants, benzodiazepines, non-benzodiazepine anxiolytics, hypnotics, anti-epileptics, herbal psychoactive compounds or monoamine oxidase inhibitors within 14 days of screening were also excluded. Patients treated with fluoxetine within 4 weeks of screening were excluded as well. Patients were also excluded for the following: (1) co-morbid anxiety disorders or dysthymia if symptoms dominated the clinical picture for ≧6 months; (2) Montgomery-Asberg Depression Rating Scale total score of >18; and (3) the presence of other clinically significant medical conditions, laboratory or ECG abnormalities.

Results

Primary analyses were based on the intention to treat (ITT) population, which included all patients who received at least one dose of study medication and had at least one post baseline efficacy evaluation. The last observation carried forward (LOCF) method was employed to analyze baseline comparisons and changes from baseline to endpoint for all efficacy variables.

The primary outcome measure was the effect of quetiapine compared with placebo on anxiety symptoms, assessed by change from baseline in HAM-A total score. Both the quetiapine and placebo group experienced a decrease in HAM-A total scores from baseline to week 6. This difference was not significant. The mean decrease from baseline in HAM-A total score for the quetiapine group as about 12 while the mean decrease from baseline for the placebo group was about 9. However, observed case analysis at Weeks 2 and 4 showed significant improvement in the quetiapine group versus the placebo group; at Week 2 (—11.1 vs. −5.9) and at Week 4 (−13.7 vs. −8.6).

Patients in the quetiapine group demonstrated greater improvement in all the other outcome measures compared to patients in the placebo group in Week 6, although the differences were not statistically significant. In contrast, the observed cases showed that the reduction in HAM-A psychic anxiety subscale was significantly better in the quetiapine group than the placebo group at Week 2 (−6.6 vs. −3.0) and at Week 4 (−7.8 vs. −4.0). The quetiapine group also showed a greater reduction from baseline on: (1) the HAM-A somatic subscale (−5.2 vs. −4.4); (2) the Hospital Anxiety and Depression (HAD) anxiety subscale (−3.79 vs. −3.16); and (3) the CGI-S (−1.37 vs. −1.05).

The response rate and remission rate at endpoint were numerically greater in the quetiapine group than in the placebo group (57.9% vs. 36.8% and 42.1% vs. 21.1% of patients, respectively). These differences, however, were not statistically significant.

Adverse Events and Tolerability

The most common AEs experienced were fatigue and somnolence. There was no statistically significant difference between groups, and those that occurred were mostly mild to moderate in severity. 12/19 patients in the quetiapine group and 16/19 patients in the placebo group completed the 6-week trial. Five patients in the quetiapine group and 2 patients in the placebo group discontinued treatment because of AEs. One patient in each group was lost to follow-up and 1 patient in quetiapine treatment withdrew their consent. In addition, no statistically significant differences between quetiapine and placebo groups were observed in AIMS, SAS, or BARS scores.

Although the study population was of limited size, the results obtained suggest evidence of improvements demonstrated by atypical antipsychotics in the treatment of GAD or treatment-resistant GAD. Quetiapine was generally well tolerated.

### Example 26

### Quetiapine and SSRIs

Anxiety disorders and depression often occur in the same individual; this leads to increased morbidity and a more prolonged course of illness. SSRIs are now first-line therapy for depression. However, these agents do not always give adequate symptom relief in patients with comorbid anxiety. Traditional antipsychotics have been used to treat anxiety and depression, but their side effect profiles have limited their use. Recent studies of quetiapine demonstrate a lack of extrapyramidal symptoms (EPS) and insignificant weight gain, which make it an ideal candidate for investigation in patients with residual anxiety and depression despite SSRI treatment.

This study was a 9-week, open-label, flexible dose study. All 11 patients were over 18 years of age and had been undergoing SSRI therapy for at least 6 weeks. They all had a DSM-IV diagnosis of unipolar depression or dysthymia and/or GAD, panic, or specific phobia; and Hamilton Anxiety Scale (HAM-A) score≧16 and State Anxiety Inventory (SAI) score≧40. SSRI doses were not optimized prior to trial. Patients with thought disorder or cognitive problems preventing informed consent, a history of significant renal, hepatic, respiratory, cardiovascular, or cerebral vascular disease were excluded. Patients who were at significant risk for suicide or had significant psychoactive use disorder within the previous 6 months were also excluded. In addition, women who were pregnant or nursing, or of child bearing age, not using an adequate method of birth control were excluded. Patients taking benzodiazepines (other than lorazepam on an as-needed basis) were not allowed to participate. Baseline evaluations of HAM-A scores and Hamilton Depression Scale (HAM-D) scores were performed. Patients were also assessed for abnormal motor movements with the SAS and the BARS. Follow-up visits were conducted at Weeks 1, 2, 3, 4, 5, 7, and 9. The starting dose of quetiapine was 25 mg/day at bedtime; this was increased as needed in 25 mg increments every 2 or 3 doses. The maximum dose of quetiapine was 100 mg in the morning and 200 mg at bedtime. Doses were titrated for the first 3 weeks, then doses were held constant until completion of the study. One patient withdrew from the study after 4 weeks; his withdrawal was not related to an adverse event.

The mean length of prior SSRI treatment for the study population was about 2 years, and paroxetine was the most common SSRI used by the group. After 9 weeks of treatment, mean HAM-A scores improved from a baseline of about 25 to about 6. Mean HAM-D scores improved from a baseline of about 20 to about 6, and mean SAI scores improved from a baseline of about 51 to about 30. A response was seen in the mean scores of all 3 assessments after Week 1. In addition, HAM-A individual subjects analysis revealed that patients saw ≧50% reduction in scores in those particular subjects. Similar reductions were also seen in HAM-D scores with regards to depressed mood, feelings of guilt, insomnia, work and activities, etc. These responses were observed as early as 2 weeks into quetiapine therapy.

Adverse Events and Tolerability

No serious adverse events occurred and no patient discontinued therapy as a result of an adverse event. In general, those that did occur were mild and transient. Drowsiness, mild dry mouth, and constipation were the most common side effects. The drowsiness was transient, and was mainly limited to the dose escalation period. There were no clinically relevant changes in the BARS or SAS scores and weight gain was minimal for all patients but one.

Addition of quetiapine to a stable dose of an SSRI resulted in a significant improvement of symptoms of anxiety and depression. This study further supports the safety and efficacy of atypical antipsychotics in augmenting the effects of antidepressants. More specifically, quetiapine may provide a better option than other atypical antipsychotics because of the decreased potential of EPS, weight gain, and prolactin elevation associated with it. Moreover, quetiapine did not increase the EPS which SSRIs are often associated with. Quetiapine does not appear to inhibit the cytochrome P450 metabolism of SSRIs, thereby reducing the potential for fluctuations in SSRI serum levels. The findings of this study indicate that quetiapine may be useful in reducing symptoms of anxiety in patients receiving stable doses of SSRIs.

### Example 27

### Quetiapine and Treatment Resistant OCD

Many patients with obsessive-compulsive disorder (OCD) are resistant to treatment with serotonin reuptake inhibitors (SRIs). Evidence suggests that, in such cases, augmentation with an atypical antipsychotic may be a treatment option.

This study was an 8-week open-label clinical trial involving 16 outpatient adults with a primary diagnosis of OCD for at least 1 year and whose symptoms did not improve after treatment with an SRI after 8 or more weeks of treatment. Five of these subjects had also failed 1 adequate trial of atypical antipsychotic augmentation, and 3 subjects had failed 2 adequate trials. An adequate augmentation trial with an atypical antipsychotic was defined as 2 or more weeks on olanzapine or risperidone. All patients had a baseline Yale-Brown Obsessive-Compulsive Scale (Y-BOCS) score≧20 (or ≧10 if obsession alone). Pregnant or nursing women or those of childbearing age who did not use a medically accepted contraceptive method were excluded. Also excluded were those subjects with organic mental disorders, psychosis, mental retardation or developmental disabilities, substance abuse or dependence within the last 6 months (excluding alcohol), depressive disorders with current suicidal risk, personality disorders, history of bipolar I or II disorders, or serious medical disorders. Those who required psychotropic medications other than an SRI or were taking a medication that may have interacted with quetiapine were excluded as well. Significant abnormalities revealed by pre-study physical examination, ECG, or laboratory test results provided additional grounds for exclusion.

Patients received 25 mg of quetiapine daily in addition to their existing medication regimen. The dosage of quetiapine was doubled every 2 weeks to a maximum of 200 mg/day depending on tolerability and effectiveness. Efficacy was assessed based upon the Y-BOCS and Montgomery-Asberg Depression Rating Scale (MADRS). Patients were evaluated at baseline and then after 1, 2, 4, 6, and 8 weeks of treatment. A response was defined as a ≧25% decrease in Y-BOCS score at endpoint.

Results

Fourteen patients completed the study. One withdrew for sedation and lack of efficacy and one was disqualified for a protocol violation. The final mean quetiapine dosage was about 169 mg/day for 2 weeks; twelve of the 16 patients received this dose. Five of the 16 patients responded to treatment. The mean Y-BOCS score of the 5 patients that responded, improved from a baseline of about 30 to about 17. These results indicate that improvements in Y-BOCS scores were independent of comorbid mood disorders. In addition, mean MADRS scores improved from a baseline of about 19 to about 15 at trial end. There was no significant correlation between the improvements in Y-BOCS and MADRS scores.

Adverse Events and Tolerability

Adverse events experienced were generally mild. Sedation and fatigue were the most common and only 1 patient withdrew because of an adverse event.

The results of this study resemble those obtained from investigations of other atypical antipsychotics and suggest that quetiapine is effective and well-tolerated as an augmenting agent for SRI-resistant OCD. Although similar studies of quetiapine have shown higher response rates, comparisons to this study lack significance because of differences in patient characteristics and in trial design. This study was limited by sample size and its open-label, non-blinded design. Thus, large-scale, double-blind, placebo-controlled trials, of longer duration should be conducted to further investigate the efficacy of quetiapine as an augmenting agent in the treatment of SSRI-resistant OCD.

### Example 28

### Quetiapine as Add-On Therapy in Psychotic Depression

Quetiapine has many similarities to clozapine. Both placebo-controlled and comparative studies in patients with schizophrenia have demonstrated that quetiapine has long-term efficacy in both positive and negative domains, as well as beneficial effects on affective and cognitive symptoms. Comparative clinical studies confirm that quetiapine is at least as effective as the standard antipsychotics, chlorpromazine and haloperidol and response rates with quetiapine are similar to those reported with other atypical antipsychotics. Quetiapine has also demonstrated superior efficacy to haloperidol in partially responsive patients, who can be particularly difficult to treat. It has a wide clinical dosage range (150-750 mg/day), with doeses of 400 mg/day and above being used in patients who do not fully respond to lower doses of the drug. It is generally well tolerated with no requirement for routine ECG or blood monitoring and it has minimal effects on weight. It is also well tolerated and effective in patients who are particularly susceptible to extrapyramidal symptoms (EPS).

This study compared the efficacy and safety of quetiapine (50-750 mg/day) as add-on therapy to citalopram (20-60 mg/day) in patients aged between 18 and 70 years, suffering from psychotic depression. To be included in the study, a patient's baseline HAM-D21 score had to be ≧25. Women of child-bearing age could be included only if their pregnancy status was assessed by the investigator prior to entry and then on a monthly basis. Female patients who were pregnant, lactating or at risk for pregnancy, were excluded. Also, those who participated in any drug trial or compassionate use program within 4 weeks of the baseline visit were not allowed to participate. Applicants were also excluded if they had: (1) a known or suspected hypersensitivity to quetiapine; (2) significant clinical, laboratory or ECG findings that would interfere with the evaluation of efficacy and tolerability; (3) another Axis 1 psychiatric diagnosis; (4) a history of non-compliance; (5) a drug dependence within the past year; or (6) a medical history of convulsive disorders, organic brain disorders, head trauma or suspected organic brain disease, severe allergic reactions, or suicidal ideations. The investigator also had complete discretion to exclude patients for any other reason if he believed the patient would not be able to complete the study per protocol. Patients could be withdrawn from the study at any time at the discretion of the investigator and were also free to discontinue participation in the study at any time without prejudice to further treatment.

The primary efficacy variable was change from baseline on the Hamilton Depression Scale (HAM-D21). Brief Psychiatric Rating Scale (BPRS) and CGI were secondary variables. Safety was assessed through documentation of adverse events (AEs), clinical laboratory data, vital signs, EEG, ECG, the Neurological Rating Scale (NRS) of Simpson and Angus, and the UKU scale.

Assessments were made at baseline and at Weeks 2, 4, and 6. Twenty-five patients were included in the study. Patients included in the study exhibited symptoms of uni- and bipolar psychotic disorder as measured by DSM-IV criteria.

Results

The mean decrease from baseline in HAM-D21 scores was about 21 (from about 31 to about 10) after 6 weeks of combination therapy. The mean decrease in BPRS score from baseline to Week 6 was about 28 (from about 59 to about 3 1). At the start of the study, all 24 patients had CGI severity scores between 5 and 7; at study end, only 1 patient was rated severity 6 and 4 patients were rated severity 5.

Safety and Tolerability

There were no serious adverse events (SAEs) reported, but 12 patients experienced at least 1 AE. Twenty-five AEs were mild and 9 were moderate. The most frequently reported AEs were gastro-intestinal system disorders, central & peripheral nervous system disorders, and liver and biliary system disorders. It is not clear whether these AEs were study-related or not. The most frequently reported possible related AEs were gastro-intestinal disorders and central and peripheral nervous system disorders.

The combination of quetiapine and citalopram was efficacious in treating psychotic depression as measured by HAM-D21, BPRS, and CGI rating scales. The combination was well tolerated and the few side effects that did occur were generally mild. Mean weight gain was <1 kg, and most abnormal initial values for serum prolactin, EPS, or UKU side effects normalized by the end of the study.

### Example 29

### Quetiapine as an Adjunt to Cognitive Behavior Therapy

Twenty to 40% of depressed patients fail to respond to standard antidepressant treatment or treatment specific psychotherapy such as Cognitive Behavior Therapy (CBT).

This study assessed the efficacy of CBT, alone and in combination with the atypical antipsychotic quetiapine in patients who suffered from refractory depression (RD). RD as defined by this study is failure of 2 or more 8-week treatments with 2 different classes of antidepressants administered at maximum doses for at least 3 of the 8 weeks. The antidepressants on which patients failed, came from a variety of classes included SSRIs, MAOs, NSRIs, and atypical antidepressants. Patients who participated in the study all met DSM-IV criteria for unipolar major depression with a minimum Hamilton Depression Scale (HAM-D) score of 20 at Day 1 and of 18 at Days 21 and 28. A CGI severity scale score of 4 or more at Days 1 and 28 was also required. Patients who suffered from comorbid conditions such as personality, anxiety, psychotic, or drug abuse disorders were excluded.

Efficacy was primarily evaluated in terms of the Montgomery Asberg Depression Rating Scale (MADRS) and the HAM-D scale. Efficacy was also assessed using the CGI severity scale and the patient related Hospital Anxiety and Depression Scale (HADS).

The trial began with a 3-week open phase augmentation with at least 600 mg/day of lithium carbonate. Thirty patients entered this phase of the study. Patients who responded to lithium treatment with at least a 40% reduction (or score<18) on the HAM-D scale were classified as responders, and excluded from the remainder of the study. The 22 patients who remained after the open phase were given no medication for 7 days and were then randomized to either CBT+placebo or CBT+quetiapine. Mean baseline values for HADS, HAMD, MADRS, and CGI were similar between the quetiapine and placebo groups. In addition, the mean duration of depression for both groups was about 2 years. CBT was administered in 12 weekly sessions given in an individual setting by the same therapist. CBT was mainly based on the Beck-Emery model of cognitive modification with applied relaxation training. Medication was administered orally with an initial dose of 12.5 mg twice a day titrated up to a maximum dose of 200 mg twice a day within the first 14 days after Day 28.

Results

CBT significantly reduced all primary efficacy measures by approximately 25% in the 22 patients that remained after the open-phase of the study. More specifically, it was after Day 70 of CBT that patients experienced a statistically significant improvement. All primary efficacy measures were significantly reduced in the CBT+quetiapine group whereas no significant reductions were observed in the CBT+placebo group. In addition, on the CGI-S scale, no patient in the placebo group achieved a score of 2 or less. Also, patients in the CBT+quetiapine group showed increased study survivability, defined as the patients having received at least 4 or more CBT sessions prior to withdrawal.

Adverse Events and Tolerability

Quetiapine was generally well-tolerated and exhibited an adverse event profile from mild to moderate. No serious adverse events were observed. Somnolence was the most common side effect.

The results of this study suggest that both lithium augmentation and CBT represent viable clinical strategies in patients with RD. It also demonstrated that the adjunctive administration of quetiapine substantially enhanced the effectiveness of CBT also. In addition, there was no lack of response drop-outs in the quetiapine group, compared to a 50% drop-out rate in the CBT+placebo arm.

### Example 30

### Quetiapine/Sertraline Combination in PTSD

Post-Traumatic Stress Disorder (PTSD) is a serious, complex and chronic mental illness, which develops mostly within 3 months following exposure to a severely stressful event. The primary aim of treatment is to stabilize the major symptoms. SSRIs are considered first-line therapy for PTSD. Although atypical antipsychotics have shown efficacy in the treatment of other anxiety disorders, studies in patients with PTSD are limited.

This study was an 8-week, randomized, placebo-controlled, double-blind study designed to assess the efficacy and tolerability of quetiapine as adjunctive therapy to an SSRI in patients with PTSD. To be included, patients had to be diagnosed with PTSD according to DSM-IV criteria. Patients with comorbid psychotic disorders or substance abuse problems were excluded. In addition, patients with severe or chronic illness, abnormal laboratory results, pregnant or lactating women, and those applicants who had been treated with SSRIs or benzodiazepines during the 2 weeks prior to the study were excluded as well. Patients were given either sertraline+quetiapine or sertraline+placebo. The dose of quetiapine was initiated at 25 mg/day. Doses were then titrated in daily increments of 25-50 mg as tolerated to a target dose of 100 mg, 200 mg, and 300 mg/day by Weeks 1, 2, and 3 respectively. The dose was split between morning and night time so that the majority of the dose was taken at bedtime. From Weeks 4 to 8, patients were given a flexible dosing regimen with a minimum dose of 200 mg/day and a maximum dose of 750 mg/day. Sertraline was initiated at 50 mg/day, and was then increased to a target of 100 mg/day at the end of the first week. The maximum dose of sertraline was 200 mg/day.

The primary efficacy endpoints were: (1) change from baseline to endpoint in the Clinician Administered PTSD Scale (CAPS) total score; and (2) the percentage of patients meeting PTSD diagnostic criteria at endpoint. Change from baseline to endpoint on the Hamilton Rating Scale for Depression (HAM-D) and CGI-S and CGI-I scores were evaluated as secondary efficacy endpoints. Adverse events (AEs) and patient adherence were also evaluated. Forty-seven patients were included in each treatment group. Demographic and baseline characteristics were similar for each group.

Results

The mean improvement (decrease) from baseline to endpoint in CAPS total score was significantly greater in the sertraline+quetiapine group than in the sertraline+placebo group (decrease of about 70 vs. decrease of about 53). After 8 weeks of treatment only 5.3% of patients in the sertraline+quetiapine group still met PTSD diagnostic criteria compared to 37% of patients in the sertraline+placebo group. This disparity was not seen in a comparison of the sertraline+quetiapine group and sertraline+placebo group when last observation carried forward (LOCF) analysis was used to analyze the results. Analysis of secondary efficacy endpoints showed that mean HAM-D scores and CGI-S scores decreased significantly from baseline to endpoint in both treatment groups. In addition, both groups showed a significant increase in CGI-I scores. Of the 59 patients in the study that experienced at least one AE, 31 were in the sertraline+placebo group and more patients in the sertraline+placebo group discontinued treatment due to AEs. The most common side effects in the sertraline+quetiapine group were drowsiness, nausea, and dry mouth.

At endpoint, the proportion of patients meeting PTSD diagnosis criteria was significantly lower with sertraline+quetiapine than with sertraline+placebo. Clinical improvements in the sertraline+quetiapine group were achieved without any impact on tolerability or patient adherence to treatment. The results of this study show that the addition of quetiapine to sertraline in the treatment of PTSD may provide more benefit than treatment with sertraline alone.

### Example 31

### Quetiapine as Monotherapy for Social Anxiety Disorder

Social Anxiety Disorder (SAD), also known as social phobia, is characterized by an overwhelming fear of social performance situations. It is one of the most common anxiety disorders with a lifetime prevalence of up to 16% in the community. It is typically a condition of lifelong duration and is commonly associated with socioeconomic disadvantages and an impaired quality of life. Response to traditional treatments used for SAD is at best moderate and recent studies have shown that quetiapine may be beneficial in patients with SAD.

This study was an 8-week, randomized, placebo-controlled, double-blind, preliminary study of quetiapine (50-400 mg/day) as monotherapy in the treatment of SAD. The study included male and female patients with a mean age of about 33. Patients had a primary diagnosis of SAD (DSM-IV), a minimum CGI-S score of 4 at baseline and a minimum Brief Social Phobia Scale (BSPS) score of 20 at baseline. Pregnant women were excluded. Dosing was titrated depending on efficacy and tolerability in each patient. Patients were started on quetiapine 25 mg twice a day for the first 3 days, and then were given 50 mg twice daily until the end of Week 1. The dose was increased to 100 mg twice a day in Week 2, then increased to 150 twice a day in Week 3, and increased to 200 mg twice daily in Week 4. Patients were assessed at baseline prior to being randomized to either the quetiapine or placebo arm. They were then assessed for efficacy and tolerability at Weeks 1, 3, 5, and 8. Primary efficacy endpoints included: (1) mean change in BSPS scores from baseline to endpoint; (2) mean improvement in CGI-I score; and (3) CGI-I response rate (defined as a CGI-I score of 1 or 2). In addition, Social Phobia Inventory (SPIN) and Sheehan Disability Inventory (SDI) scores, evaluated from baseline to endpoint, were used as secondary efficacy measures.

Fifteen patients were enrolled in the study; 10 received active drug while 5 received placebo. General characteristics of both groups were similar (i.e., age, ethnicity, marital status, and gender). The mean final dose of quetiapine was 147 mg twice a day.

Results

There were no significant differences in BSPS scores between the quetiapine and placebo groups at baseline or endpoint. However, the percentage of patients experiencing a ≧50% drop in BSPS score at endpoint compared with baseline was greater in the quetiapine group as compared to the placebo group (20% vs. 0%). There was no significant difference between quetiapine and placebo in mean CGI-I scores at endpoint; nor was there any significant difference in CGI-I responders versus CGI-I non-responders within or between the treatment groups. However, a greater percentage of patients in the quetiapine group scored a 1 or 2 on the CGI-I scale as compared to the placebo group (40% vs. 0%). In terms of secondary outcomes, there was no significant difference in SPIN or SDI scores between the two groups at baseline or endpoint. No serious adverse events were reported with quetiapine or placebo. The most frequent side effects that occurred in the quetiapine group were drowsiness, dizziness, and nausea. The results of this study show that quetiapine improved symptoms of SAD compared to placebo.

### Example 32

### Quetiapine Combination for Treatment-Resistant Depression

MDD is a very common and untreated condition which results in a high degree of disability. MDD is also associated with a high-degree of treatment-resistance. SSRIs and SNRIs are first-line therapy. Those who do not respond are left with the option of switching to another drug, or combining therapy with a drug from another class. Growing evidence supports the use of atypical antipsychotics such as quetiapine in treatment-resistant depression.

This study was an 8-week, double-blind, randomized, placebo-controlled outpatient investigation of patients with a primary diagnosis of major depression who were not psychotic and had a baseline Hamilton Depression (HAM-D) score≧20 following at least 6 weeks of treatment with an SSRI or SNRI. Patients who had a substance abuse or dependence problem within 3 months of the start of the study, or failed a urine test for illicit substances were excluded. Those judged to be a serious suicidal or homicidal risk and those who had a history of clinically significant disease that would affect or be affected by trial medication were also excluded. Pregnant or lactating women, or those who were planning on becoming pregnant were not allowed to participate either. Participation in a clinical research study within 90 days of the start of this study also precluded applicants from participating. In addition, those who had received treatment with mood stabilizers, other antipsychotics or antidepressants other than SSRIs/SNRIs for a minimum of 2 weeks prior to enrollment were excluded.

Patients were randomized to receive quetiapine (200-400 mg/day) or placebo in addition to ongoing SSRI/SNRI treatment. Quetiapine was initiated at a dose of 50 mg once daily at bedtime. The dose was increased by 50 mg every 3 days to a minimum target level of 200 mg/day, up to a total maximum dose of 600 mg/day. The primary efficacy endpoint for this study was HAM-D score at Week 8. Secondary endpoints included: (1) response (≧50% reduction in HAM-D score) and remission (HAM-D score<7) rates; (2) final Montgomery-Asberg Rating Scale score (MADRS); (3) CGI-S scores; and (4) CGI-I scores. Efficacy was assessed at baseline, then at Weeks 1, 2, 3, 4, 6, and 8. Adverse events (AEs) were also monitored.

Results

Combination antidepressant-quetiapine therapy resulted in significantly lower mean HAM-D scores compared to placebo at study end (about 8 in the quetiapine group and about 15 in the placebo group). The quetiapine group also showed a significantly lower mean MADRS score at study end compared to placebo (about 15 in the quetiapine group and about 24 in the placebo group). Final mean CGI-S and CGI-I scores were also significantly lower at the end of the study in the quetiapine group than in the placebo group (2.8 vs 3.8 and 2.5 vs. 3.5 respectively). In addition, significantly more patients in the quetiapine group responded to treatment as compared to the placebo group (67% vs. 27%) and 43% of the patients in the quetiapine group achieved remission while only 15% of the patients in the placebo group did the same. Quetiapine was generally well-tolerated. The most common side effects observed in the quetiapine group were fatigue, dry mouth, and sedation/somnolence. These side effects are no different than those seen in other clinical trials of quetiapine. No patients in the quetiapine group withdrew from the study due to serious adverse events, while 2 patients in the placebo arm withdrew because of panic attacks and sedation. Patients in the quetiapine group did experience a mean weight gain of about 6 kg. Extrapyramidal symptoms (EPS) were evaluated on three different scales and the mean scores for all three scales were similar at baseline and at study end for both groups.

Quetiapine in combination with an SSRI/SNRI was effective and generally well-tolerated in patients with treatment-resistant depression. The improvements in both HAM-D and MADRS scores for the quetiapine group suggest that patients realized a genuine improvement in symptoms aside from the sedative effect of quetiapine.

### Example 33

### Quetiapine Augmentation of SSRIs/SNRIs in Major Depression with Anxiety

Although SSRIs and SNRIs are first-line therapy for major depression, they have a few disadvantages. In addition, many patients are left with residual depressive symptoms despite being treated. Atypical antidepressants such as quetiapine are widely used in clinical practice for the augmentation of antidepressant therapy.

This study was double-blind, randomized study which evaluated quetiapine augmentation of SSRIs/SNRIs in patients with major depression who also had prominent anxiety symptoms. Fifty-eight patients with residual symptoms of depression and anxiety following at least 6 weeks of treatment received quetiapine (50-600 mg/day) or placebo for 8 weeks. The primary efficacy endpoints were change from baseline in Hamilton Rating Scale for Depression (HAM-D) and HAM-A scores. Patients were also evaluated according to the CGI-S scale for severity, the Global Assessment scale (GAS) and for adverse events (AEs). Patients were evaluated at baseline and at Weeks 1, 2, 4, 6, and 8. Response was defined as a ≧50% reduction in HAM-D or HAM-A total scores from baseline to Week 8. Remission was defined as a HAM-D or HAM-A total score of 7 or lower at Week 8.

In order to be included in the study, patients had to have a DSM-IV diagnosis of major depression (HAM-D score≧18), a CGI-S score≧4, and a HAM-A score≧14. Patients also had to have been treated with a SSRI or SNRI for at least 6 weeks prior to the start of the study. Patients who had: (1) a current CNS disorder; (2) significant hepatic, renal or gastrointestinal impairment; (3) acute, unstable or significant and untreated medical conditions; (4) current substance abuse or dependence; or (5) were at risk for suicide were excluded. Pregnant or breast-feeding women were excluded also. The mean age of patients in both groups was about 45 and the 2 groups were generally well-matched in terms of baseline characteristics other than mean body weight. The quetiapine group began the study with a mean body weight 7 kg less than the placebo group. The mean quetiapine dose was 202 mg/day for those who completed the study. Thirty-four patients completed the study; 18 in the quetiapine group and 16 in the placebo group. The majority of patients who did not complete the study in the quetiapine group did so because of the AEs of somnolence and sedation. However, no serious AEs were reported, and those that were reported were similar to the AEs seen in other clinical trials of quetiapine. An increase in body weight≧5 kg did occur in 6 patients enrolled in the quetiapine group, while no patients in the placebo group experienced weight gain. Those who quit the placebo group did so mainly because of a lack of efficacy.

Results

The mean improvement from baseline in HAM-D scores was about 11 (from about 23 to about 12) in the quetiapine group as compared to an improvement from baseline of about 5 (from about 23 to about 18) in the placebo group. Similarly, the mean improvement from baseline in HAM-A scores was about 13 (from about 23 to about 10) in the quetiapine group as compared to only an improvement from baseline of about 5 (from about 23 to about 18) in the placebo group. Response rates for quetiapine were greater than for placebo as assessed by HAM-D and HAM-A scores (48% vs. 28% and 62% vs. 28% respectively). Remission rates as assessed by HAM-D and HAM-A scores were also higher in the quetiapine group (31% vs. 17% and 41% vs. 17% respectively). CGI-S and GAS assessments also showed that quetiapine was significantly more effective than placebo.

Quetiapine augmentation of SSRI and SNRI therapy reduced symptoms of depression and anxiety in patients with MDD, comorbid anxiety, and residual depressive symptoms. The combination was generally well-tolerated and there were no unexpected tolerability issues.

### Example 34

### Add-On Queitapine in the Treatment of MDD in Elderly Patients with CV Damage

Depression is a common risk for elderly patients and is associated with an elevated risk of mortality. Affective disorders (such as depression) and vascular disease are frequently comorbid conditions found in the elderly. They share certain etiopathogenetic and prognostic factors and untreated depression may exacerbate vascular disease. The correlation between the 2 conditions has led to the identification of what has come to be known as “vascular depression.” Depressive episodes in elderly patients with cerebrovascular (CV) damage are characterized by low response rates to antidepressants. Therefore, investigation of new treatments is necessary.

This study investigated the effects of quetiapine as add-on therapy in 9 elderly patients (>63 years of age) with a diagnosis of MDD and CV damage (assessed by MRI) without severe cognitive impairment. It was an open-label, 6-month follow-up study of patients who had not responded to standard antidepressant treatment. The mean length of antidepressant treatment prior to the study was about 7 months. Patients were evaluated at baseline and then after 1,3, and 6 months. They were evaluated according to the Hamilton Rating Scale for Depression (HAM-D) and the CGI-S scale for severity. The mean age of patients in the study was about 73. Mean baseline HAM-D scores were about 27 and mean baseline CGI-S scores were about 6.

Quetiapine was administered as add-on therapy with commonly prescribed antidepressants (e.g., paroxetine, citalopram, sertraline, and mirtazapine). Quetiapine was initiated at a minimum daily dose of 25 mg/day on Day 1 and was titrated up to 200 mg/day on Day 7. After Day 7, the dosage was increased by 100 mg every 2 days until the optimal dose, based on individual response and tolerability, was reached. The mean quetiapine dose during the study was 300 mg/day.

Results

Mean HAM-D scores also decreased from about 27 to about 15 after 6 months. Mean CGI-S scores improved from a baseline score of about 6 to about 5 after 6 months of add-on quetiapine therapy. No patients discontinued the study. Sedation and drowsiness were the only side effects reported.

Add-on quetiapine therapy significantly improved depressive symptoms and was well tolerated in elderly patients with comorbid depression and CV damage who had previously failed to respond to standard antidepressant therapy.

Various modifications of the invention, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

What is claimed is:

1. A method of treating a patient suffering from or susceptible to a Mood Disorder or an Anxiety Disorder comprising administering a sustained release pharmaceutical composition comprising a pharmaceutically effective amount of 11-[4-[2-(2-hydroxyethoxy)ethyl]-1-piperazinyl]dibenzo-[b,f][1,4]thiazepine, or a pharmaceutically acceptable salt thereof to the patient in need thereof.

2. The method of claim 1 wherein the sustained release composition comprises a polymer.

3. The method of claims 1 or 2 wherein the patient is in need of the treatment.

4. The method of any of claims 1-3 wherein the Mood Disorder is Major Depressive Disorder.

5. The method of any of claims 1-3 wherein the Mood Disorder is Bipolar Disorder.

6. The method of any of claims 1-3 wherein the Mood Disorder is Bipolar Depression.

7. The method of any of claims 1-6 wherein the mean Cmax of the sustained release composition is from about 10 ng/mL to about 700 ng/mL and the area under the curve (AUC) is from about 100 ng*hr/mL to about 6000 ng*hr/mL.

8. The method of any of claims 1-6 wherein the median Tmax of the sustained release composition is from about 1.5 hours to about 7.5 hours.

9. The method of any of claims 1-8 wherein the treatment is monotherapy treatment with 11-[4-[2-(2-hydroxyethoxy)ethyl]-1-piperazinyl]dibenzo-[b,f][1,4]thiazepine.

10. The method of any of claims 1-8 wherein the treatment is maintenance treatment with 11-[4-[2-(2-hydroxyethoxy)ethyl]-1-piperazinyl]dibenzo-[b,f][1,4]thiazepine.

11. The method of any of claims 1-8 or 10 wherein the 11-[4-[2-(2-hydroxyethoxy)ethyl]-1-piperazinyl]dibenzo-[b,f][1,4]thiazepine is administered as part of adjunct therapy with a selective serotonin reuptake inhibitor.

12. The method of claim 11 wherein the selective serotonin reuptake inhibitor is selected from paroxetine, fluoxetine, sertaline, fluvoxamine, venlafaxine, nefazodone, and citalopram.

13. The method of claim 11 or claim 12 wherein a therapeutically effective amount of the selective serotonin reuptake inhibitor, or a pharmaceutically acceptable salt thereof, is administered in a pharmaceutical composition that comprises the sustained release composition of 11-[4-[2-(2-hydroxyethoxy)ethyl]-1-piperazinyl]dibenzo-[b,f][1,4]thiazepine, or a pharmaceutically acceptable salt thereof.

14. The method of any of claims 1-8 or 11 wherein the 11-[4-[2-(2-hydroxyethoxy)ethyl]-1-piperazinyl]dibenzo-[b,f][1,4]thiazepine is administered as part of adjunct therapy with a serotonin-norepinephrine reuptake inhibitor.

15. The method of claim 14 wherein the serotonin-norepinephrine reuptake inhibitor is duloxetine.

16. The method of claim 14 or claim 15 wherein the adjunct therapy is for treatment of Major Depressive Disorder.

17. The method of claim 14 or claim 15 wherein the adjunct therapy is for treatment of Major Depressive Disorder and comorbid anxiety.

18. A method of relieving a symptom of depression in a patient comprising administering a therapeutically effective amount of 11-[4-[2-(2-hydroxyethoxy)ethyl]-1-piperazinyl]dibenzo-[b,f][1,4]thiazepine in a sustained release composition to a patient in need thereof, wherein 11-[4-[2-(2-hydroxyethoxy)ethyl]-1-piperazinyl]dibenzo-[b,f][1,4]thiazepine is administered to the patient once daily, wherein the method excludes administering to the patient other compositions for relieving a symptom of depression.

19. A method of relieving a symptom of depression in a patient suffering from or susceptible to a Mood Disorder comprising administering a therapeutically effective amount of 11-[4-[2-(2-hydroxyethoxy)ethyl]-1-piperazinyl]dibenzo-[b,f][1,4]thiazepine in a sustained release composition to a patient in need thereof, wherein 11-[4-[2-(2-hydroxyethoxy)ethyl]-1-piperazinyl]dibenzo-[b,f][1,4]thiazepine is administered to the patient once or twice daily, wherein the 11-[4-[2-(2-hydroxyethoxy)ethyl]-1-piperazinyl]dibenzo-[b,f][1,4]thiazepine is administered as a monotherapy.

20. A method of relieving a symptom of depression in a patient suffering from or susceptible to Major Depressive Disorder comprising administering a therapeutically effective amount of 11-[4-[2-(2-hydroxyethoxy)ethyl]-1-piperazinyl]dibenzo-[b,f][1,4]thiazepine in a sustained release composition to a patient in need thereof, wherein 11-[4-[2-(2-hydroxyethoxy)ethyl]-1-piperazinyl]dibenzo-[b,f][1,4]thiazepine is administered to the patient once or twice daily, wherein the 11-[4-[2-(2-hydroxyethoxy)ethyl]-1-piperazinyl]dibenzo-[b,f][1,4]thiazepine is administered as a monotherapy.

21. A method of treating a patient suffering from or susceptible to an Anxiety Disorder comprising administering to the patient in need thereof a pharmaceutical composition comprising a pharmaceutically effective amount of 11-[4-[2-(2-hydroxyethoxy)ethyl]-1-piperazinyl]dibenzo-[b,f][1,4]thiazepine, or a pharmaceutically acceptable salt thereof.

22. The method of claim 21 wherein the 11-[4-[2-(2-hydroxyethoxy)ethyl]-1-piperazinyl]dibenzo-[b,f][1,4]thiazepine, or a pharmaceutically acceptable salt thereof, is present within a sustained release composition.

23. The method of claim 22 wherein the sustained release composition comprises a polymer.

24. The method of any of claims 21-23 wherein the patient is in need of the treatment.

25. The method of any of claims 21-24 wherein the Anxiety Disorder is Generalized Anxiety Disorder.

26. The method of any of claims 22-25 wherein the mean Cmax of the sustained release composition is from about 10 ng/mL to about 700 ng/mL and the area under the curve (AUC) is from about 100 ng*hr/mL to about 6000 ng*hr/mL.

27. The method of any of claims 22-25 wherein the median Tmax of the sustained release composition is from about 1.5 hours to about 7.5 hours.

28. The method of any of claims 21-27 wherein the treatment is monotherapy treatment with 11-[4-[2-(2-hydroxyethoxy)ethyl]-1-piperazinyl]dibenzo-[b,f][1,4]thiazepine.

29. The method of any of claims 21-28 wherein the treatment is maintenance treatment with 11-[4-[2-(2-hydroxyethoxy)ethyl]-1-piperazinyl]dibenzo-[b,f][1,4]thiazepine.

30. The method of any of claims 21-27 and 29 wherein the 11-[4-[2-(2-hydroxyethoxy)ethyl]-1-piperazinyl]dibenzo-[b,f][1,4]thiazepine is administered as part of adjunct therapy with a selective serotonin reuptake inhibitor.

31. The method of claim 30 wherein the selective serotonin reuptake inhibitor is selected from paroxetine, fluoxetine, sertaline, fluvoxamine, venlafaxine, nefazodone, and citalopram.

32. The method of claim 30 or claim 31 wherein a therapeutically effective amount of the selective serotonin reuptake inhibitor, or a pharmaceutically acceptable salt thereof, is administered in a pharmaceutical composition that comprises the sustained release composition of 11-[4-[2-(2-hydroxyethoxy)ethyl]-1-piperazinyl]dibenzo-[b,f][1,4]thiazepine, or a pharmaceutically acceptable salt thereof.

33. The method of any of claims 21-27 and 29 wherein the 11-[4-[2-(2-hydroxyethoxy)ethyl]-1-piperazinyl]dibenzo-[b,f][1,4]thiazepine is administered as part of adjunct therapy with a serotonin-norepinephrine reuptake inhibitor.

34. The method of claim 33 wherein the serotonin-norepinephrine reuptake inhibitor is duloxetine.

35. A method of relieving a symptom associated with Generalized Anxiety Disorder in a patient comprising administering a therapeutically effective amount of 11-[4-[2-(2-hydroxyethoxy)ethyl]-1-piperazinyl]dibenzo-[b,f][1,4]thiazepine in a patient in need thereof, wherein 11-[4-[2-(2-hydroxyethoxy)ethyl]-1-piperazinyl]dibenzo-[b,f][1,4]thiazepine is administered to the patient once daily, wherein the method excludes administering to the patient other compositions for relieving a symptom associated with Generalized Anxiety Disorder.

36. A method of relieving a symptom associated with Generalized Anxiety Disorder in a patient comprising administering a therapeutically effective amount of 11-[4-[2-(2-hydroxyethoxy)ethyl]-1-piperazinyl]dibenzo-[b,f][1,4]thiazepine in a patient in need thereof, wherein 11-[4-[2-(2-hydroxyethoxy)ethyl]-1-piperazinyl]dibenzo-[b,f][1,4]thiazepine is administered to the patient once or twice daily, wherein the 11-[4-[2-(2-hydroxyethoxy)ethyl]-1-piperazinyl]dibenzo-[b,f][1,4]thiazepine is administered as a monotherapy.

37. The method of claim 35 wherein the symptom treated is anxiety.

38. The method of claim 35 wherein the symptom is relapse of anxiety symptoms with Generalized Anxiety Disorder.

39. Use of a sustained release pharmaceutical composition comprising a pharmaceutically effective amount of 11-[4-[2-(2-hydroxyethoxy)ethyl]-1-piperazinyl]dibenzo-[b,f][1,4]thiazepine, or a pharmaceutically acceptable salt thereof, in the manufacture of a medicament for the treatment of a patient suffering from or susceptible to a Mood Disorder or an Anxiety Disorder.

40. The use of claim 39 wherein the sustained release composition comprises a polymer.

41. The use of claims 39 or 40 wherein the patient is in need of the treatment.

42. The use of any of claims 39-41 wherein the Mood Disorder is Major Depressive Disorder.

43. The use of any of claims 39-41 wherein the Mood Disorder is Bipolar Disorder.

44. The use of any of claims 39-41 wherein the Mood Disorder is Bipolar Depression.

45. The use of any of claims 39-44 wherein the mean Cmax of the sustained release composition is from about 10 ng/mL to about 700 ng/mL and the area under the curve (AUC) is from about 100 ng*hr/mL to about 6000 ng*hr/mL.

46. The use of any of claims 39-44 wherein the median Tmax of the sustained release composition is from about 1.5 hours to about 7.5 hours.

47. The use of any of claims 39-46 wherein the medicament is for monotherapy treatment with 11-[4-[2-(2-hydroxyethoxy)ethyl]-1-piperazinyl]dibenzo-[b,f][1,4]thiazepine.

48. The use of any of claims 39-47 wherein the medicament is for maintenance treatment with 11-[4-[2-(2-hydroxyethoxy)ethyl]-1-piperazinyl]dibenzo-[b,f][1,4]thiazepine.

49. The use of any of claims 39-46 or 48 wherein the medicament is used as part of adjunct therapy with a selective serotonin reuptake inhibitor.

50. The use of claim 49 wherein the selective serotonin reuptake inhibitor is selected from paroxetine, fluoxetine, sertaline, fluvoxamine, venlafaxine, nefazodone, and citalopram.

51. The use of claim 49 or claim 50 wherein the medicament further comprises a therapeutically effective amount of the selective serotonin reuptake inhibitor, or a pharmaceutically acceptable salt thereof.

52. The use of any of claims 39-46 or 49 wherein the medicament is used as part of adjunct therapy with a serotonin-norepinephrine reuptake inhibitor.

53. The use of claim 52 wherein the serotonin-norepinephrine reuptake inhibitor is duloxetine.

54. The use of claim 52 or claim 53 wherein the adjunct therapy is for treatment of Major Depressive Disorder.

55. The use of claim 52 or claim 53 wherein the adjunct therapy is for treatment of Major Depressive Disorder and comorbid anxiety.

56. Use of 11-[4-[2-(2-hydroxyethoxy)ethyl]-1-piperazinyl]dibenzo-[b,f][1,4]thiazepine in a sustained release composition in the manufacture of a once daily medicament for relieving a symptom of depression in a patient.

57. Use of 11-[4-[2-(2-hydroxyethoxy)ethyl]-1-piperazinyl]dibenzo-[b,f][1,4]thiazepine in a sustained release composition in the manufacture of a once-daily or twice daily monotherapy medicament for relieving a symptom of depression in a patient suffering from or susceptible to a Mood Disorder.

58. Use of 11-[4-[2-(2-hydroxyethoxy)ethyl]-1-piperazinyl]dibenzo-[b,f][1,4]thiazepine in a sustained release composition in the manufacture of a once-daily or twice daily monotherapy medicament for relieving a symptom of depression in a patient suffering from or susceptible to Major Depressive Disorder.

59. Use of 11-[4-[2-(2-hydroxyethoxy)ethyl]-1-piperazinyl]dibenzo-[b,f][1,4]thiazepine, or a pharmaceutically acceptable salt thereof, in the manufacture of a medicament for treating a patient suffering from or susceptible to an Anxiety Disorder.

60. The use of claim 59 wherein the 11-[4-[2-(2-hydroxyethoxy)ethyl]-1-piperazinyl]dibenzo-[b,f][1,4]thiazepine, or a pharmaceutically acceptable salt thereof, is present within a sustained release composition.

61. The use of claim 60 wherein the sustained release composition comprises a polymer.

62. The use of any of claims 59-61 wherein the patient is in need of the treatment.

63. The use of any of claims 59-52 wherein the Anxiety Disorder is Generalized Anxiety Disorder.

64. The use of any of claims 60-63 wherein the mean Cmax of the sustained release composition is from about 10 ng/mL to about 700 ng/mL and the area under the curve (AUC) is from about 100 ng*hr/mL to about 6000 ng*hr/mL.

65. The use of any of claims 60-63 wherein the median Tmax of the sustained release composition is from about 1.5 hours to about 7.5 hours.

66. The use of any of claims 59-65 wherein the medicament is for monotherapy treatment.

67. The use of any of claims 59-66 wherein the medicament is for maintenance treatment.

68. The use of any of claims 59-65 or 67 wherein the medicament is used a part of adjunct therapy with a selective serotonin reuptake inhibitor.

69. The use of claim 68 wherein the selective serotonin reuptake inhibitor is selected from paroxetine, fluoxetine, sertaline, fluvoxamine, venlafaxine, nefazodone, and citalopram.

70. The use of claim 68 or claim 69 wherein the medicament comprises a therapeutically effective amount of the selective serotonin reuptake inhibitor, or a pharmaceutically acceptable salt thereof.

71. The use of any of claims 59-65 or 67 wherein the medicament is used as part of adjunct therapy with a serotonin-norepinephrine reuptake inhibitor.

72. The use of claim 71 wherein the serotonin-norepinephrine reuptake inhibitor is duloxetine.

73. Use of 11-[4-[2-(2-hydroxyethoxy)ethyl]-1-piperazinyl]dibenzo-[b,f][1,4]thiazepine in the manufacture of a once-daily medicament for relieving a symptom associated with Generalized Anxiety Disorder in a patient.

74. Use of 11-[4-[2-(2-hydroxyethoxy)ethyl]-1-piperazinyl]dibenzo-[b,f][1,4]thiazepine in the manufacture of a once-daily or twice-daily monotherapy medicament for relieving a symptom associated with Generalized Anxiety Disorder in a patient.

75. The use of claim 73 wherein the symptom treated is anxiety.

76. The use of claim 73 wherein the symptom is relapse of anxiety symptom with Generalized Anxiety Disorder.
